(19) United States
(12) Patent Application Publication
(10) Pub. No.: US 2026/0306682 A1
(43) Pub. Date: Oct. 8, 2026

(54) RADIOLABELED MET BINDING PROTEINS FOR IMMUNO-PET IMAGING

(71) Applicant: Regeneron Pharmaceuticals, Inc., Tarrytown, NY (US)

(72) Inventors: Marcus Kelly, Pleasantville, NY (US); Dangshe Ma, Millwood, NY (US); William Olson, Yorktown Heights, NY (US)

(21) Appl. No.: 19/573,798

(22) Filed: Mar. 20, 2026

Related U.S. Application Data
(63) Continuation of application No. 18/400,943, filed on Dec. 29, 2023, now Pat. No. 12,605,473.
Application No. 18/400,943 is a continuation of application No. 17/021,497, filed on Sep. 15, 2020, now Pat. No. 11,896,682.
(60) Provisional application No. 62/901,003, filed on Sep. 16, 2019.

(51) Int. Cl. A61K 51/10; C07K 16/28

(52) U.S. Cl. CPC A61K 51/103; C07K 16/2863; C07K 2317/21; C07K 2317/31; C07K 2317/565; C07K 2317/77; C07K 2317/92; C07K 2317/94

(57) ABSTRACT

Radiolabeled anti-MET antibodies and MET×MET bispecific antibodies and their use in immuno-PET imaging are provided herein. Included are methods of detecting the presence of MET proteins in a subject or sample and methods of monitoring efficacy of treatment of a Met expressing tumor.

### RELATED APPLICATIONS

This application is a continuation application of U.S. patent application Ser. No. 18/400,943, filed Dec. 29, 2023, which is a continuation application of U.S. patent application Ser. No. 17/021,497, filed Sep. 15, 2020, now U.S. Pat. No. 11,896,682, which claims priority under 35 U.S.C. 119 (e) to U.S. Provisional Patent Application Ser. No. 62/901,003, entitled “RADIOLABELED MET BINDING PROTEINS FOR IMMUNO-PET IMAGING”, filed Sep. 16, 2019, the entire contents of each of which are hereby incorporated by reference herein.

### FIELD

This disclosure relates to radiolabeled MET binding proteins and their use in immuno-PET imaging.

### SEQUENCE LISTING

A copy of the sequence listing is submitted concurrently with the specification electronically via Patent Center. The content of the electronic sequence listing (10649US03_Sequence_Listing_ST26.xml; Size: 159,744 bytes; and Date of Creation: Mar. 20, 2026) is herein incorporated by reference in its entirety.

### BACKGROUND

Hepatocyte growth factor (HGF) (a.k.a. scatter factor [SF]) is a heterodimeric paracrine growth factor that exerts its activity by interacting with the HGF receptor (HGFR). HGFR is the product of the c-Met oncogene and is also known as MET. MET is a receptor tyrosine kinase consisting of a transmembrane beta chain linked via a disulfide bridge to an extracellular alpha chain. The binding of HGF to MET activates the kinase catalytic activity of MET resulting in the phosphorylation of Tyr 1234 and Tyr 1235 of the beta chain and subsequent activation of downstream signaling pathways.

MET and/or HGF overexpression, activation, or amplification has been shown to be involved in non-small cell lung carcinoma (NSCLC), gastric, ovarian, pancreatic, thyroid, breast, head and neck, colon and kidney carcinomas (Sierra and Tsao, Ther. Adv. Med. Oncol., 3 (1 Suppl): S21-S35, 2011). MET amplification is thought to be a key driver of oncogenesis in NSCLCs and oesophagogastric malignancies. In addition, mutations resulting in exon 14 deletion of MET have been described as oncogenic drivers in a subset of NSCLC. Tumor cell lines having MET gene amplification are highly dependent on MET for growth and survival. Preclinical data implicate MET signaling in resistance to targeted therapies in multiple tumor types, such as NSCLC, colorectal cancer, and head and neck squamous-cell carcinoma (HNSCC).

Immuno-positron emission tomography (PET) is a diagnostic imaging tool that utilizes monoclonal antibodies labeled with positron emitters, combining the targeting properties of an antibody with the sensitivity of positron emission tomography cameras. See, e.g., The Oncologist, 12:1379 (2007); Journal of Nuclear Medicine, 52 (8): 1171 (2011). Immuno-PET enables the visualization and quantification of antigen and antibody accumulation in vivo and, as such, can serve as an important tool for diagnostics and complementing therapy. For example, immuno-PET can aid in the selection of potential candidates for a particular therapy, as well as in the monitoring of treatment.

Both preclinical and recent clinical results indicate that tumors harboring MET genetic alterations respond to MET inhibitors, validating MET as a cancer driver. As such, there is need for diagnostic tools for anti-MET and/or anti-MET therapy, including, inter alia, diagnostic tools that enable the detection of suitable candidates for said therapy.

### BRIEF SUMMARY

Included in this disclosure are radiolabeled anti-MET antibody conjugates and MET×MET bispecific antibody conjugates for use in immuno-PET imaging.

In one aspect, the conjugate comprises an anti-MET antibody, a MET×MET bispecific antibody, or an antigen-binding fragment thereof, a chelating moiety, and a positron emitter.

Provided herein are also processes for synthesizing said conjugates and synthetic intermediates useful for the same.

Provided herein are also methods of imaging a tissue that expresses MET, the methods comprising administering a radiolabeled anti-MET antibody conjugate or MET×MET bispecific antibody conjugate described herein to the tissue; and visualizing the MET expression by positron emission tomography (PET) imaging.

Provided herein are also methods for detecting MET in a tissue, the methods comprising administering a radiolabeled anti-MET antibody conjugate or MET×MET bispecific antibody conjugate described herein to the tissue; and visualizing the MET expression by PET imaging. In one embodiment, the tissue is present in a human subject. In certain embodiments, the subject is a non-human mammal. In certain embodiments, the subject has a disease or disorder such as cancer.

Provided herein are also methods for determining the presence of MET expressing cells in a subject. The methods comprise administering a radiolabeled anti-MET antibody conjugate or MET×MET bispecific antibody conjugate described herein to the subject and visualizing MET expression by PET imaging.

Provided herein are also methods for identifying a subject having a solid tumor to be suitable for anti-tumor therapy comprising an inhibitor of the HGF/MET signaling pathway, for example, an anti-MET antibody, a MET×MET bispecific antibody, or an antibody drug conjugate (ADC) thereof. The methods comprise administering a radiolabeled antibody conjugate described herein to the subject, and visualizing the administered radiolabeled antibody conjugate in the tumor by PET imaging wherein presence of the radiolabeled antibody conjugate in the tumor identifies the subject as suitable for anti-tumor therapy comprising an inhibitor of the HGF/MET signaling pathway.

Provided herein are also methods of treating a solid tumor in a subject, the methods comprising determining that the solid tumor is MET-positive; and administering an anti-tumor therapy to the subject in need thereof. In certain embodiments, the anti-tumor therapy comprises an anti-MET antibody or a MET×MET bispecific antibody. In certain embodiments, the subject is administered a radiolabeled antibody conjugate described herein, and localization of the radiolabeled antibody conjugate is imaged via positron emission tomography (PET) imaging to determine if the tumor is MET-positive.

Provided herein are also methods for monitoring the efficacy of an anti-tumor therapy in a subject being treated with an anti-tumor therapy, wherein the methods comprise administering a radiolabeled conjugate described herein to the subject; imaging the localization of the administered radiolabeled conjugate in the tumor by PET imaging; and determining tumor growth, wherein a decrease from the baseline in uptake of the conjugate or radiolabeled signal indicates tumor regression and efficacy of the anti-tumor therapy. In certain embodiments, the anti-tumor therapy comprises an inhibitor of the HGF/MET signaling pathway (e.g., an anti-MET antibody or a MET×MET bispecific antibody, or an ADC of either).

Provided herein are also methods for predicting response of a subject to an anti-tumor therapy comprising an inhibitor of the HGF/MET signaling pathway, the methods comprising determining if the tumor is MET-positive, wherein if the tumor is MET-positive it indicates a positive response of the subject to an anti-tumor therapy comprising an inhibitor of the HGF/MET signaling pathway. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is MET-positive.

Provided herein are methods for diagnosing and treating a subject with a tumor, the methods comprising administering a radiolabeled conjugate described herein to the subject wherein localization of the radiolabeled antibody conjugate is imaged via PET imaging to determine if the tumor is MET-positive; diagnosing the subject with a MET-positive tumor; and administering to the subject an anti-tumor therapy comprising an inhibitor of the HGF/MET signaling pathway.

Provided herein are methods for diagnosing a subject having a MET expressing tumor, the methods comprising administering a radiolabeled anti-MET antibody conjugate or MET×MET bispecific antibody conjugate described herein to the subject; visualizing MET expression by PET imaging; and diagnosing the subject with a MET expressing tumor when MET expression is visualized by PET imaging.

### BRIEF DESCRIPTION OF THE FIGURES

FIG. 1 depicts an SE-HPLC chromatogram of a 5 μg injection of DFO-MET×MET immunoconjugate conjugate on Superdex 200 Increase column with UV 280 nm absorbance detection. Monomeric (99.6%) and high molecular weight (HMW) species (0.4%) are indicated.

FIG. 2 depicts an image of SDS-PAGE of the DFO-MET×MET immunoconjugate. The gel demonstrates that the antibody integrity remains unchanged after DFO conjugation. Lanes are labeled as follows: 1) Standard ladder (BioRad, Cat. #: 161-0374), 2) bispecific antibody non-reduced, 3) DFO-Ab immunoconjugate non-reduced, 4) blank, 5) bispecific antibody reduced, 6) DFO-Ab immunoconjugate non-reduced. Each well was loaded with approximately 2 μg of protein. Note that non-reduced antibodies typically demonstrate less electrophoretic motility than expected as compared to the ladder for the standard SDS-PAGE setup.

FIG. 3 depicts a representative SE-HPLC radiochromatogram of a 5 μg injection of a radioimmunoconjugate (DFO-MET×MET bispecific antibody) with gamma emission detection. The RCP of is greater than 95% while unincorporated ⁸⁹Zr makes up less than 1% of total integrated activity.

FIG. 4 depicts a representative SE-HPLC UV absorption chromatogram of a 5 μg injection of a radioimmunoconjugate (DFO-MET×MET bispecific antibody). Main (97.9%) and HMW (2.1%) species are indicated. The elution peaks between 25 and 31 minutes is a phenomenon of formulation buffer/mobile phase mixing and is deemed not proteinaceous in origin.

FIG. 5 depicts PET/CT images of EBC-1 tumor xenografts in mice. The mice were administered radiolabeled MET×MET bispecific antibody conjugate and over several days, the conjugate specifically localized to the MET expressing tumor xenografts.

FIG. 6 depicts PET/CT images of NCI-H441 tumor xenografts in mice. The mice were administered radiolabeled MET×MET bispecific antibody conjugate and over several days, the conjugate specifically localized to the MET expressing tumor xenografts.

FIG. 7 depicts PET/CT images of NCI-H358 tumor xenografts in mice. The mice were administered radiolabeled MET×MET bispecific antibody conjugate and over several days, the conjugate specifically localized to the MET expressing tumor xenografts.

FIG. 8A, FIG. 8B, FIG. 8C, FIG. 8D, FIG. 8E, and FIG. 8F provide ex vivo biodistribution data for ⁸⁹Zr-DFO-MET×MET bispecific antibody conjugate in SCID mice with tumor xenografts. Mice were administered a single IV dose 0.1 mg/kg, 0.5 mg/kg, or 5.0 mg/kg ⁸⁹Zr-DFO-MET×MET bispecific antibody conjugate and were sacrificed 6 days later. Blood, collected via cardiac puncture, and the indicated harvested tissues were weighed and radioactivity was determined. The percent injected dose per gram (% ID/g) values for individual samples collected on day 6 were calculated relative to the radioactivity of a dose-standard from injected material (⁸⁹Zr-DFO-MET×MET bispecific antibody conjugate) and the weight of the individual samples. Data are plotted as mean±SD.

FIG. 9 shows the correlation between uptake of ⁸⁹Zr-DFO-MET×MET bispecific antibody and MET expression level in the tumor xenografts from three MET expressing cell lines.

FIG. 10A and FIG. 10B show antibody saturation binding data for three MET expressing cell lines.

### DETAILED DESCRIPTION

### I. Definitions

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise herein, all technical and scientific terms used herein have the same meaning as commonly understood by one of ordinary skill in the art to which the disclosed subject matter belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Met Protein

The expressions “MET,” “c-Met,” and the like, as used herein, refer to the human membrane spanning receptor tyrosine kinase comprising (1) the amino acid sequence as set forth in SEQ ID NO:145, and/or having the amino acid sequence as set forth in NCBI accession No. NM_001127500.2, representing the unprocessed preproprotein of isoform “a”, (2) the amino acid sequence as set forth in SEQ ID NO:146, and/or having the amino acid sequence as set forth in NCBI accession No. NM_000236.2, representing the unprocessed preproprotein of isoform “b”, (3) the amino acid sequence as set forth in SEQ ID NO: 147, and/or having the amino acid sequence as set forth in NCBI accession No. NM_001311330.1, representing the unprocessed preproprotein of isoform “c”, and/or (3) the mature protein comprising the cytoplasmic alpha subunit (SEQ ID NO:148) shared by all three isoforms and the transmembrane beta subunit (SEQ ID NO:149, 150, or 151 of isoform a, b and c, respectively). The expression “MET” includes both monomeric and multimeric MET molecules. As used herein, the expression “monomeric human MET” means a MET protein or portion thereof that does not contain or possess any multimerizing domains and that exists under normal conditions as a single MET molecule without a direct physical connection to another MET molecule. An exemplary monomeric MET molecule is the molecule referred to herein as “hMET.mmh” comprising the amino acid sequence of SEQ ID NO: 152 (see, e.g., Example 3 of US-2018-0134794). As used herein, the expression “dimeric human MET” means a construct comprising two MET molecules connected to one another through a linker, covalent bond, non-covalent bond, or through a multimerizing domain such as an antibody Fc domain. An exemplary dimeric MET molecule is the molecule referred to herein as “hMET.mFc” comprising the amino acid sequence of SEQ ID NO:153 (see, e.g., Example 3 of US-2018-0134794).

All references to proteins, polypeptides and protein fragments herein are intended to refer to the human version of the respective protein, polypeptide or protein fragment unless explicitly specified as being from a non-human species. Thus, the expression “MET” means human MET unless specified as being from a non-human species, e.g., “mouse MET,” “monkey MET,” etc.

As used herein, the expression “cell surface-expressed MET” means one or more MET protein(s), or the extracellular domain thereof, that is/are expressed on the surface of a cell in vitro or in vivo, such that at least a portion of a MET protein is exposed to the extracellular side of the cell membrane and is accessible to an antigen-binding portion of an antibody. A “cell surface-expressed MET” can comprise or consist of a MET protein expressed on the surface of a cell which normally expresses MET protein. Alternatively, “cell surface-expressed MET” can comprise or consist of MET protein expressed on the surface of a cell that normally does not express human MET on its surface but has been artificially engineered to express MET on its surface.

### Other Definitions

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The human anti-MET antibodies or MET×MET bispecific antibodies useful herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences of Table 1 to germline sequences available from, for example, public antibody sequence databases. Useful according to the present disclosure are antibodies, and antigen-binding fragments thereof, which are derived from any of the amino acid sequences provided in Table 1, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences according to Table 1, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

Useful herein are MET binding proteins such as human anti-MET antibodies and MET×MET bispecific antibodies comprising variants of any of the HCVR, LCVR, and/or CDR amino acid sequences shown in Table 1 herein having one or more conservative substitutions. For example, the present disclosure includes MET×MET bispecific antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences of Table 1.

The term “human antibody”, as used herein, is intended to include antibodies having variable and constant regions derived from human germline immunoglobulin sequences. The human monoclonal antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include monoclonal antibodies in which CDR sequences derived from the germline of another mammalian species (e.g., mouse), have been grafted onto human FR sequences.

The term “multi-specific antigen-binding molecules”, as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multi-specific antigen-binding molecules” also includes bi-specific, tri-specific or multi-specific antibodies or antigen-binding fragments thereof. In certain embodiments, an antibody of the present disclosure is functionally linked to another antibody or antigen-binding fragment thereof to produce a bispecific antibody with a second binding specificity. Bispecific and multi-specific antibodies of the present disclosure are described elsewhere herein.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to MET. Moreover, multi-specific antibodies that bind to one domain in MET and one or more additional antigens or a bi-specific that binds to two different regions of MET are nonetheless considered antibodies that “specifically bind”, as used herein.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to MET.

An “isolated antibody”, as used herein, is intended to refer to an antibody that is substantially free of other antibodies (Abs) having different antigenic specificities (e.g., an isolated antibody that specifically binds MET, or a fragment thereof, is substantially free of Abs that specifically bind antigens other than MET.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D} “, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix. Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, in need of amelioration, prevention and/or treatment of a disease or disorder such as cancer.

### II. Radiolabeled Immunoconjugates of MET Antibodies for Immuno-PET Imaging

Provided herein are radiolabeled antigen-binding proteins that bind MET protein. In some embodiments, the radiolabeled antigen-binding proteins comprise an antigen-binding protein covalently linked to one or more chelating moieties, which are chemical moieties that are capable of chelating a positron emitter.

In some embodiments, provided herein are antigen-binding proteins that bind MET, e.g., anti-MET antibodies or MET×MET bispecific antibodies, wherein said antigen-binding proteins that bind MET are covalently bonded to one or more moieties having the following structure:

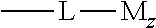

wherein L is a chelating moiety; M is a positron emitter; and z, independently at each occurrence, is 0 or 1; and wherein at least one of z is 1.

In some embodiments, the radiolabeled antigen-binding protein is a compound of Formula (I):

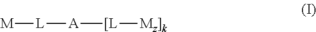

A is a protein that binds MET; L is a chelating moiety; M is a positron emitter; z is 0 or 1; and k is an integer from 0-30. In some embodiments, k is 1.

In certain embodiments, the radiolabeled antigen-binding protein is a compound of Formula (II):

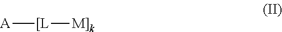

wherein A is a protein that binds MET; L is a chelating moiety; M is a positron emitter; and k is an integer from 1-30.

In some embodiments, provided herein are compositions comprising a conjugate having the following structure:

A-Lₖ

wherein A is a protein that binds MET; L is a chelating moiety; and k is an integer from 1-30; wherein the conjugate is chelated with a positron emitter in an amount sufficient to provide a specific activity suitable for clinical PET imaging.

Suitable binding proteins, chelating moieties, and positron emitters are provided below.

### A. MET Binding Proteins

Suitable MET binding protein are proteins that specifically bind to MET, including those described in U.S. Patent Publication No. 2018-0134794, incorporated herein by reference in its entirety. Amino acid sequence identifiers of exemplary anti-MET antibodies useful herein are listed in Table 1 of U.S. Patent Publication No. 2018-0134794 and amino acid sequence identifiers of exemplary MET×MET bispecific antibodies useful herein are listed in Table 5 of U.S. Patent Publication No. 2018-0134794. Both Tables are included below as Tables 1 and 2, respectively.

TABLE 1
Amino Acid Sequence Identifiers

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H13290P2 | 2 | 4 | 6 | 8 | 138 | 140 | 142 | 144 |
| H4H13291P2 | 10 | 12 | 14 | 16 | 138 | 140 | 142 | 144 |
| H4H13295P2 | 18 | 20 | 22 | 24 | 138 | 140 | 142 | 144 |
| H4H13299P2 | 26 | 28 | 30 | 32 | 138 | 140 | 142 | 144 |
| H4H13300P2 | 34 | 36 | 38 | 40 | 138 | 140 | 142 | 144 |
| H4H13301P2 | 42 | 44 | 46 | 48 | 138 | 140 | 142 | 144 |
| H4H13302P2 | 50 | 52 | 54 | 56 | 138 | 140 | 142 | 144 |
| H4H13306P2 | 58 | 60 | 62 | 64 | 138 | 140 | 142 | 144 |
| H4H13309P2 | 66 | 68 | 70 | 72 | 138 | 140 | 142 | 144 |
| H4H13311P2 | 74 | 76 | 78 | 80 | 138 | 140 | 142 | 144 |
| H4H13312P2 | 82 | 84 | 86 | 88 | 138 | 140 | 142 | 144 |
| H4H13313P2 | 90 | 92 | 94 | 96 | 138 | 140 | 142 | 144 |
| H4H13316P2 | 98 | 100 | 102 | 104 | 138 | 140 | 142 | 144 |
| H4H13318P2 | 106 | 108 | 110 | 112 | 138 | 140 | 142 | 144 |
| H4H13319P2 | 114 | 116 | 118 | 120 | 138 | 140 | 142 | 144 |
| H4H13325P2 | 122 | 124 | 126 | 128 | 138 | 140 | 142 | 144 |
| H4H13331P2 | 130 | 132 | 134 | 136 | 138 | 140 | 142 | 144 |

TABLE 2
MET × MET Bispecific Antibody Components Summary

| | SEQ ID NOs: (Amino Acid Sequences) | | | | | | | |
|---|---|---|---|---|---|---|---|---|
| | First Antigen-Binding | | | | Second Antigen-Binding | | | |
| | Domain (D1) | | | | Domain (D2) | | | |
| Bispecific | D1- | D1- | D1- | D1- | D2- | D2- | D2- | D2- |
| Antibody | HCVR | HCDR1 | HCDR2 | HCDR3 | HCVR | HCDR1 | HCDR2 | HCDR3 |
| H4H14634D | H4H13290P2 | | | | H4H13312P2 | | | |
| (No. 10) | 2 | 4 | 6 | 8 | 82 | 84 | 86 | 88 |
| H4H14635D | H4H13295P2 | | | | H4H13312P2 | | | |
| (No. 42) | 18 | 20 | 22 | 24 | 82 | 84 | 86 | 88 |
| H4H14636D | H4H13299P2 | | | | H4H13312P2 | | | |
| (No. 74) | 26 | 28 | 30 | 32 | 82 | 84 | 86 | 88 |
| H4H14637D | H4H13301P2 | | | | H4H13312P2 | | | |
| (No. 90) | 42 | 44 | 46 | 48 | 82 | 84 | 86 | 88 |
| H4H14638D | H4H13302P2 | | | | H4H13312P2 | | | |
| (No. 106) | 50 | 52 | 54 | 56 | 82 | 84 | 86 | 88 |
| H4H14639D | H4H13306P2 | | | | H4H13312P2 | | | |
| (No. 122) | 58 | 60 | 62 | 64 | 82 | 84 | 86 | 88 |
| H4H14640D | H4H13309P2 | | | | H4H13312P2 | | | |
| (No. 138) | 66 | 68 | 70 | 72 | 82 | 84 | 86 | 88 |
| H4H14641D | H4H13313P2 | | | | H4H13312P2 | | | |
| (No. 187) | 90 | 92 | 94 | 96 | 82 | 84 | 86 | 88 |
| H4H16445D | H4H13291P2 | | | | H4H13312P2 | | | |
| (No. 26) | 10 | 12 | 14 | 16 | 82 | 84 | 86 | 88 |
| H4H16446D | H4H13300P2 | | | | H4H13312P2 | | | |
| (No. 58) | 34 | 36 | 38 | 40 | 82 | 84 | 86 | 88 |
| H4H16447D | H4H13311P2 | | | | H4H13312P2 | | | |
| (No. 154) | 74 | 76 | 78 | 80 | 82 | 84 | 86 | 88 |
| H4H16448D | H4H13318P2 | | | | H4H13312P2 | | | |
| (No. 219) | 106 | 108 | 110 | 112 | 82 | 84 | 86 | 88 |
| H4H16449D | H4H13319P2 | | | | H4H13312P2 | | | |
| (No. 235) | 114 | 116 | 118 | 120 | 82 | 84 | 86 | 88 |

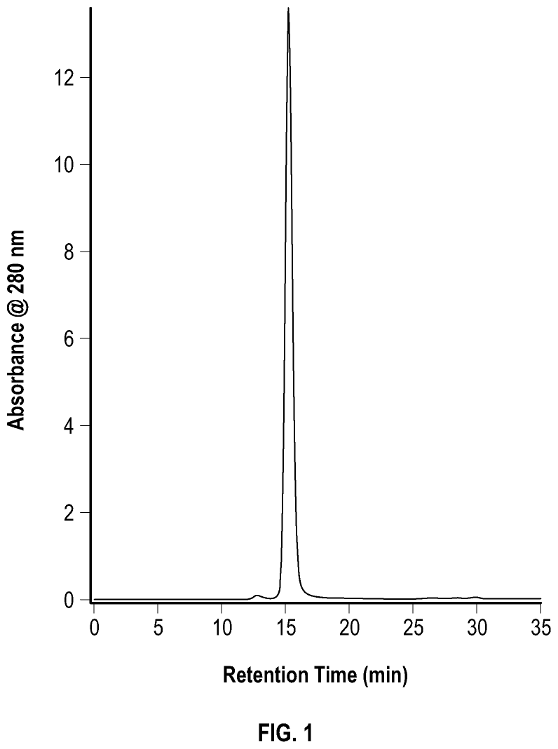

* The number designation in parentheses under the bispecific antibody identifiers (e.g., “No. 10”) indicates the bispecific antibody number depicted in the MET × MET bispecific antibody matrix of U.S. Patent Publication No. 2018-0134794, FIG. 1.

Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-MET antibodies.

In some embodiments, the binding protein is an antibody or antigen binding fragment comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

In some embodiments, the binding protein is an antibody or antigen binding fragment comprising an LCVR amino acid sequence shown in Table 1, or a substantially similar sequence thereof having at least 90%, at least 95%, at least 98% or at least 99% sequence identity thereto.

In some embodiments, the binding protein is an antibody or antigen binding fragment comprising an HCVR and an LCVR amino acid sequence pair (HCVR/LCVR) comprising any of the HCVR amino acid sequences listed in Table 1 paired with the LCVR amino acid sequence shown in Table 1. According to certain embodiments, the present disclosure provides antibodies, or antigen-binding fragments thereof, comprising an HCVR/LCVR amino acid sequence pair contained within any of the exemplary anti-MET antibodies listed in Table 1. In certain embodiments, the HCVR/LCVR amino acid sequence pair is selected from the group consisting of SEQ ID NOs: 2/138, 10/138, 18/138, 26/138, 34/138, 42/138, 50/138, 58/138, 66/138, 74/138, 82/138, 90/138, 98/138,106/138, 114/138, 122/138, and 130/138. In certain embodiments, the HCVR/LCVR amino acid sequence pair is selected from one of SEQ ID NOs: 58/138 (e.g., H4H13306P2) and 82/138 (e.g., H4H13312P2).

In some embodiments, the binding protein is an antibody or antigen binding fragment comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR1 (LCDR1) comprising an amino acid sequence shown in Table 1 or a substantially similar sequence thereof having at least 90%, at least 95%, at least 98% or at least 99% sequence identity.

In some embodiments, the binding protein is an antibody or antigen binding fragment comprising a light chain CDR2 (LCDR2) comprising an amino acid sequence shown in Table 1 or a substantially similar sequence thereof having at least 90%, at least 95%, at least 98% or at least 99% sequence identity.

In some embodiments, the binding protein is an antibody or antigen binding fragment comprising a light chain CDR3 (LCDR3) comprising an amino acid sequence shown in Table 1 or a substantially similar sequence thereof having at least 90%, at least 95%, at least 98% or at least 99% sequence identity.

In some embodiments, the binding protein is an antibody or antigen binding fragment comprising an HCDR3 and an LCDR3 amino acid sequence pair (HCDR3/LCDR3) comprising any of the HCDR3 amino acid sequences listed in Table 1 paired with the LCDR3 amino acid sequences shown in Table 1. According to certain embodiments, the present disclosure provides antibodies, or antigen-binding fragments thereof, comprising an HCDR3/LCDR3 amino acid sequence pair contained within any of the exemplary anti-MET antibodies listed in Table 1. In certain embodiments, the HCDR3/LCDR3 amino acid sequence pair is selected from the group consisting of SEQ ID NOs: 8/144 (e.g. H4H13290P2), 16/144 (e.g. H4H13291P2), 24/144 (H4H13295P2), 32/144 (H4H13299P2), 40/144 (H4H13300P2), 48/144 (H4H13301P2), 56/144 (H4H13302P2), 64/144 (H4H13306P2), 72/144 (H4H13309P2), 80/144 (H4H13311P2), 88/144 (H4H13312P2), 96/144 (H4H13313P2), 104/144 (H4H13316P2), 112/144 (H4H13318P2), 120/144 (H4H13319P2), 128/144 (H4H13325P2), and 136/144 (H4H13331P2).

In some embodiments, the binding protein is an antibody or antigen binding fragment comprising a set of six CDRs (i.e., HCDR1-HCDR2-HCDR3-LCDR1-LCDR2-LCDR3) contained within any of the exemplary anti-MET antibodies listed in Table 1. In certain embodiments, the HCDR1-HCDR2-HCDR3-LCDR1-LCDR2-LCDR3 amino acid sequence set is selected from the group consisting of SEQ ID NOs: 4-6-8-140-142-144 (e.g. H4H13290P2), Dec. 14, 2016-140-142-144 (e.g. H4H13291P2), 20-22-24-140-142-144 (H4H13295P2), 28-30-32-140-142-144 (H4H13299P2), 36-38-40-140-142-144 (H4H13300P2), 44-44-48-140-142-144 (H4H13301P2), 52-54-56-140-142-144 (H4H13302P2), 60-62-64-140-142-144 (H4H13306P2), 68-70-72-140-142-144 (H4H13309P2), 76-78-80-140-142-144 (H4H13311P2), 84-86-88-140-142-144 (H4H13312P2), 92-94-96-140-142-144 (H4H13313P2), 100-102-104-140-142-144 (H4H13316P2), 108-110-112-140-142-144 (H4H13318P2), 116-118-120-140-142-144 (H4H13319P2), 124-126-128-140-142-144 (H4H13325P2), and 132-134-136-140-142-144 (H4H13331P2).

In some embodiments, the binding protein is an antibody or antigen binding fragment comprising a set of six CDRs (i.e., HCDR1-HCDR2-HCDR3-LCDR1-LCDR2-LCDR3) contained within an HCVR/LCVR amino acid sequence pair as defined by any of the exemplary anti-MET antibodies listed in Table 1. For example, in some embodiments, the binding protein is an antibody or antigen binding fragment comprising the HCDR1-HCDR2-HCDR3-LCDR1-LCDR2-LCDR3 amino acid sequences set contained within an HCVR/LCVR amino acid sequence pair selected from the group consisting of SEQ ID NOS: 2/138 (e.g. H4H13290P2), 10/138 (e.g. H4H13291P2), 18/138 (H4H13295P2), 26/138 (H4H13299P2), 34/138 (H4H13300P2), 42/138 (H4H13301P2), 50/138 (H4H13302P2), 58/138 (H4H13306P2), 66/138 (H4H13309P2), 74/138 (H4H13311P2), 82/138 (H4H13312P2), 90/138 (H4H13313P2), 98/138 (H4H13316P2), 106/138 (H4H13318P2), 114/138 (H4H13319P2), 122/138 (H4H13325P2), and 130/138 (H4H13331P2).

Methods and techniques for identifying CDRs within HCVR and LCVR amino acid sequences are well known in the art and can be used to identify CDRs within the specified HCVR and/or LCVR amino acid sequences useful herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In some embodiments, binding proteins are antibodies and antigen-binding fragments thereof that compete for specific binding to MET with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

Table 2 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the first antigen-binding domains (D1) and second antigen-binding domains (D2) of several exemplary MET×MET bispecific antibodies.

The individual anti-MET antigen-binding domains used to construct the bispecific antibodies useful herein were derived from various bivalent, monospecific anti-MET antibodies described in Examples 1 through 3 of U.S. Publication No. 2018-0134794. All anti-MET antibodies described herein comprise the same (“common”) light chain (comprising the light chain variable region [LCVR] amino acid sequence of SEQ ID NO:138, and light chain CDR [LCDR1, LCDR2 and LCDR3] amino acid sequences of SEQ ID NOS: 140, 142 and 144). In addition, all of the bispecific antibodies contain a “D2” arm derived from the exemplary anti-MET antibody H4H13312P2. Thus, both antigen-binding domains (D1 and D2) of all of the bispecific antibodies described in this example comprise this common light chain variable region, and all D2 binding arms comprise the heavy chain variable region from H4H13312P2; however, the bispecific antibodies differ from one another in terms of their D1 heavy chain variable regions (HCVRs) and heavy chain CDRs (HCDRs). D1 and D2 are derived from different anti-MET antibodies and, consequently, bind to separate epitopes on the MET extracellular domain. I.e., D1 can bind a first epitope of human MET, e.g. an epitope comprising amino acids 192-204 of SEQ ID NO: 155, and D2 can bind a second epitope of human MET comprising amino acids 305-315 and 421-455 of SEQ ID NO:155.

As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., human MET). The term “specifically binds” or the like, as used herein, means that the antigen-binding domain forms a complex with a particular antigen characterized by a dissociation constant (K_{D}) of 500 μM or less, and does not bind other unrelated antigens under ordinary test conditions. “Unrelated antigens” are proteins, peptides or polypeptides that have less than 95% amino acid identity to one another.

Exemplary categories of antigen-binding domains that can be used in the context of the present disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding domain, as used in the context of the present disclosure, includes polypeptides that bind a particular antigen (e.g., a target molecule [T] or an internalizing effector protein [E]) or a portion thereof with a K_{D} of less than about 500 μM, less than about 400 μM, less than about 300 μM, less than about 200 pM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 4 μM, less than about 2 μM, less than about 1 μM, less than about 0.5 μM, less than about 0.2 μM, less than about 0.1 μM, or less than about 0.05 μM, as measured in a surface plasmon resonance assay.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D} “, as used herein, means the equilibrium dissociation constant of a particular protein-protein interaction (e.g., antibody-antigen interaction). Unless indicated otherwise, the K_{D} values exhibited by the antibodies useful herein refer to K_{D} values determined by surface plasmon resonance assay at 25° C. or 37° C.

As indicated above, an “antigen-binding domain” (D1 and/or D2) may comprise or consist of an antibody or antigen-binding fragment of an antibody. The term “antibody,” as used herein, means any antigen-binding molecule or molecular complex comprising at least one complementarity determining region (CDR) that specifically binds to or interacts with a particular antigen (e.g., human MET). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the antibodies provided herein (or antigen-binding portion thereof) may be identical to the human germline sequences, or may be naturally or artificially modified. An amino acid consensus sequence may be defined based on a side-by-side analysis of two or more CDRs.

The D1 and/or D2 components of the bispecific antigen-binding molecules useful herein may comprise or consist of antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In some embodiments, the binding protein is a bispecific antigen-binding molecule comprising or consisting of human antibodies and/or recombinant human antibodies, or fragments thereof. The term “human antibody”, as used herein, includes antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Methods for making bispecific antibodies are known in the art and may be used to construct bispecific antigen-binding molecules useful in the conjugates described herein. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats).

Exemplary antigen-binding domains (D1 and D2) that can be included in the MET×MET bispecific antigen-binding molecules useful herein include antigen-binding domains derived from any of the anti-MET antibodies disclosed in Table 1. For example, the present disclosure includes MET×MET bispecific antigen-binding molecules comprising a D1 or D2 antigen-binding domain comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The binding protein can be a MET×MET bispecific antigen-binding molecule comprising a D1 or D2 antigen-binding domain comprising an LCVR comprising an amino acid sequence shown in Table 1, or a substantially similar sequence thereof having at least 90%, at least 95%, at least 98% or at least 99% sequence identity thereto.

In some embodiments, the binding protein is a MET×MET bispecific antigen-binding molecule comprising a D1 or D2 antigen-binding domain comprising an HCVR and an LCVR amino acid sequence pair (HCVR/LCVR) comprising any of the HCVR amino acid sequences listed in Table 1 paired with the LCVR amino acid sequence shown in Table 1.

In some embodiments, the binding protein is a MET×MET bispecific antigen-binding molecule comprising a D1 or D2 antigen-binding domain comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

In some embodiments, the binding protein is a MET×MET bispecific antigen-binding molecule comprising a D1 or D2 antigen-binding domain comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

In some embodiments, the binding protein is a MET×MET bispecific antigen-binding molecule comprising a D1 or D2 antigen-binding domain comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

In some embodiments, the binding protein is a MET×MET bispecific antigen-binding molecule comprising a D1 or D2 antigen-binding domain comprising a light chain CDR1 (LCDR1) comprising an LCDR1 amino acid sequence shown in Table 1 or a substantially similar sequence thereof having at least 90%, at least 95%, at least 98% or at least 99% sequence identity.

In some embodiments, the binding protein is a MET×MET bispecific antigen-binding molecule comprising a D1 or D2 antigen-binding domain comprising a light chain CDR2 (LCDR2) comprising an LCDR2 amino acid sequence shown in Table 1 or a substantially similar sequence thereof having at least 90%, at least 95%, at least 98% or at least 99% sequence identity.

In some embodiments, the binding protein is a MET×MET bispecific antigen-binding molecule comprising a D1 or D2 antigen-binding domain comprising a light chain CDR3 (LCDR3) comprising an LCDR3 amino acid sequence shown in Table 1 or a substantially similar sequence thereof having at least 90%, at least 95%, at least 98% or at least 99% sequence identity.

In some embodiments, the binding protein is a MET×MET bispecific antigen-binding molecule comprising a D1 or D2 antigen-binding domain comprising an HCDR3 and an LCDR3 amino acid sequence pair (HCDR3/LCDR3) comprising any of the HCDR3 amino acid sequences listed in Table 1 paired with the LCDR3 amino acid sequence shown in Table 1.

In some embodiments, the binding protein is a MET×MET bispecific antigen-binding molecule comprising a D1 or D2 antigen-binding domain comprising a set of six CDRs (i.e., HCDR1-HCDR2-HCDR3-LCDR1-LCDR2-LCDR3) contained within any of the exemplary anti-MET antibodies listed in Table 1.

In some embodiments, the binding protein is a MET×MET bispecific antigen-binding molecule comprising a D1 or D2 antigen-binding domain comprising a set of six CDRs (i.e., HCDR1-HCDR2-HCDR3-LCDR1-LCDR2-LCDR3) contained within an HCVR/LCVR amino acid sequence pair as defined by any of the exemplary anti-MET antibodies listed in Table 1.

The MET×MET bispecific antigen-binding molecules useful herein may comprise a D1 antigen-binding domain derived from any of the anti-MET antibodies of Table 1, and a D2 antigen-binding domain derived from any other anti-MET antibody of Table 1. Non-limiting examples of MET×MET bispecific antibodies of the present disclosure are depicted in FIG. 1 of U.S. Patent Publication No. 2018-0134794, which illustrates the components of 272 exemplary MET×MET bispecific antibodies. Each numbered cell of the matrix (numbered 1 through 272) identifies a unique bispecific antibody comprising a “D1” antigen binding domain and a “D2” antigen binding domain, wherein the D1 antigen binding domain comprises the immunoglobulin variable domain (HCVR/LCVR amino acid sequence pair) or CDRs from the corresponding anti-MET antibody listed along the Y-axis, and wherein the D2 antigen binding domain comprises the immunoglobulin variable domain (HCVR/LCVR amino acid sequence pair) or CDRs from the corresponding anti-MET antibody listed along the X-axis. Thus, for example, the MET×MET bispecific antigen-binding molecule “number 10” shown in the matrix comprises a D1 antigen-binding domain comprising an HCVR/LCVR pair, or 6-CDR set, from the exemplary anti-MET antibody H4H13290P2, and a D2 antigen-binding domain comprising an HCVR/LCVR pair, or 6-CDR set, from the exemplary anti-MET antibody H4H13321P2. Additional examples of MET×MET bispecific antibodies provided herein are described in Example 4 of U.S. Patent Publication No. 2018-0134794.

In some embodiments, the binding protein is a MET×MET bispecific antigen binding molecule comprising a D1 antigen-binding domain and a D2 antigen-binding domain, wherein the D1 antigen binding domain comprises an HCVR/LCVR amino acid sequence pair of SEQ ID NOs: 58/138, or a substantially similar sequence thereof having at least 90%, at least 95%, at least 98% or at least 99% sequence identity, or a set of heavy and light chain CDRs (HCDR1-HCDR2-HCDR3-LCDR1-LCDR2-LCDR3) comprising SEQ ID NOs: 60-62-64-140-142-144, or a substantially similar sequence thereof having at least 90%, at least 95%, at least 98% or at least 99% sequence identity, and wherein the D2 antigen-binding domain comprises an HCVR/LCVR amino acid sequence pair of SEQ ID NOs: 82/138, or a substantially similar sequence thereof having at least 90%, at least 95%, at least 98% or at least 99% sequence identity, or a set of heavy and light chain CDRs (HCDR1-HCDR2-HCDR3-LCDR1-LCDR2-LCDR3) comprising SEQ ID NOs: 84-86-88-140-142-144, or a substantially similar sequence thereof having at least 90%, at least 95%, at least 98% or at least 99% sequence identity.

In some embodiments, the binding protein is a MET×MET bispecific antigen binding molecule comprising a D1 antigen-binding domain and a D2 antigen-binding domain, wherein the D1 antigen binding domain comprises an HCVR/LCVR amino acid sequence pair of SEQ ID NOs: 18/138, or a substantially similar sequence thereof having at least 90%, at least 95%, at least 98% or at least 99% sequence identity, or a set of heavy and light chain CDRs (HCDR1-HCDR2-HCDR3-LCDR1-LCDR2-LCDR3) comprising SEQ ID NOs: 20-22-24-140-142-144, or a substantially similar sequence thereof having at least 90%, at least 95%, at least 98% or at least 99% sequence identity, and wherein the D2 antigen-binding domain comprises an HCVR/LCVR amino acid sequence pair of SEQ ID NOs: 82/138, or a substantially similar sequence thereof having at least 90%, at least 95%, at least 98% or at least 99% sequence identity, or a set of heavy and light chain CDRs (HCDR1-HCDR2-HCDR3-LCDR1-LCDR2-LCDR3) comprising SEQ ID NOs: 84-86-88-140-142-144, or a substantially similar sequence thereof having at least 90%, at least 95%, at least 98% or at least 99% sequence identity.

In some embodiments, the binding protein is a MET×MET bispecific antigen binding molecule comprising a D1 antigen-binding domain and a D2 antigen-binding domain, wherein the D1 antigen binding domain comprises an HCVR/LCVR amino acid sequence pair of SEQ ID NOs: 58/138, or a set of heavy and light chain CDRs (HCDR1-HCDR2-HCDR3-LCDR1-LCDR2-LCDR3) comprising SEQ ID NOs: 60-62-64-140-142-144, and wherein the D2 antigen-binding domain comprises an HCVR/LCVR amino acid sequence pair of SEQ ID NOs: 82/138, or a set of heavy and light chain CDRs (HCDR1-HCDR2-HCDR3-LCDR1-LCDR2-LCDR3) comprising SEQ ID NOs: 84-86-88-140-142-144. An exemplary MET×MET bispecific antibody having these sequence characteristics is the bispecific antibody designated H4H14639D, also referred to as bispecific antibody No. 122, which comprises a D1 derived from H4H13306P2 and a D2 derived from H4H13312P2 (see Table 2 herein).

In some embodiments, the binding protein is a MET×MET bispecific antigen binding molecule comprising a D1 antigen-binding domain and a D2 antigen-binding domain, wherein the D1 antigen binding domain comprises an HCVR/LCVR amino acid sequence pair of SEQ ID NOs: 18/138, or a set of heavy and light chain CDRs (HCDR1-HCDR2-HCDR3-LCDR1-LCDR2-LCDR3) comprising SEQ ID NOs: 20-22-24-140-142-144, and wherein the D2 antigen-binding domain comprises an HCVR/LCVR amino acid sequence pair of SEQ ID NOs: 82/138, or a set of heavy and light chain CDRs (HCDR1-HCDR2-HCDR3-LCDR1-LCDR2-LCDR3) comprising SEQ ID NOs: 84-86-88-140-142-144. An exemplary MET×MET bispecific antibody having these sequence characteristics is the bispecific antibody designated H4H14635D, also referred to as bispecific antibody No. 42, which comprises a D1 derived from H4H13295P2 and a D2 derived from H4H13312P2 (see Table 2 herein).

The bispecific antigen-binding molecules useful herein may also comprise one or more multimerizing component(s). The multimerizing components can function to maintain the association between the antigen-binding domains (D1 and D2). As used herein, a “multimerizing component” is any macromolecule, protein, polypeptide, peptide, or amino acid that has the ability to associate with a second multimerizing component of the same or similar structure or constitution. For example, a multimerizing component may be a polypeptide comprising an immunoglobulin C_{H}3 domain. A non-limiting example of a multimerizing component is an Fc portion of an immunoglobulin, e.g., an Fc domain of an IgG selected from the isotypes IgG1, IgG2, IgG3, and IgG4, as well as any allotype within each isotype group. In certain embodiments, the multimerizing component is an Fc fragment or an amino acid sequence of 1 to about 200 amino acids in length containing at least one cysteine residues. In other embodiments, the multimerizing component is a cysteine residue, or a short cysteine-containing peptide. Other multimerizing domains include peptides or polypeptides comprising or consisting of a leucine zipper, a helix-loop motif, or a coiled-coil motif.

In certain embodiments, the bispecific antigen-binding molecules useful herein comprise two multimerizing domains, M1 and M2, wherein D1 is attached to M1 and D2 is attached to M2, and wherein the association of M1 with M2 facilitates the physical linkage of D1 and D2 to one another in a single bispecific antigen-binding molecule. In certain embodiments, M1 and M2 are identical to one another. For example, M1 can be an Fc domain having a particular amino acid sequence, and M2 is an Fc domain with the same amino acid sequence as M1. Alternatively, M1 and M2 may differ from one another at one or more amino acid position. For example, M1 may comprise a first immunoglobulin (lg) C_{H}3 domain and M2 may comprise a second Ig C_{H}3 domain, wherein the first and second Ig C_{H}3 domains differ from one another by at least one amino acid, and wherein at least one amino acid difference reduces binding of the targeting construct to Protein A as compared to a reference construct having identical M1 and M2 sequences. In one embodiment, the Ig C_{H}3 domain of M1 binds Protein A and the Ig C_{H}3 domain of M2 contains a mutation that reduces or abolishes Protein A binding such as an H95R modification (by IMGT exon numbering; H435R by EU numbering). The C_{H}3 of M2 may further comprise a Y96F modification (by IMGT; Y436F by EU). Further modifications that may be found within the C_{H}3 of M2 include: D16E, L18M, N44S, K52N, V57M, and V821 (by IMGT; D356E, L358M, N384S, K392N, V397M, and V4221 by EU) in the case of an IgG1 Fc domain; N44S, K52N, and V821 (IMGT; N384S, K392N, and V4221 by EU) in the case of an IgG2 Fc domain; and Q15R, N44S, K52N, V57M, R69K, E79Q, and V821 (by IMGT; Q355R, N384S, K392N, V397M, R409K, E419Q, and V4221 by EU) in the case of an IgG4 Fc domain.

In some embodiments, the binding protein may be “isolated.” An “isolated bispecific antigen-binding molecule,” as used herein, means a bispecific antigen-binding molecule that has been identified and separated and/or recovered from at least one component of its natural environment. For example, a bispecific antibody that has been separated or removed from at least one component of an organism, or from a tissue or cell in which the antibody is produced, is an “isolated bispecific antibody” for purposes of the present disclosure. An isolated bispecific antigen-binding molecule also includes molecules in situ within a recombinant cell. Isolated bispecific antigen-binding molecules are molecules that have been subjected to at least one purification or isolation step. According to certain embodiments, an isolated bispecific antigen-binding molecule may be substantially free of other cellular material and/or chemicals.

The bispecific antigen-binding molecules useful herein, or the antigen-binding domains thereof (D1 and/or D2) may comprise one or more amino acid substitutions, insertions and/or deletions in the framework and/or CDR regions of the heavy and light chain variable domains as compared to the corresponding germline sequences from which the antigen-binding proteins or antigen-binding domains were derived. Such mutations can be readily ascertained by comparing the amino acid sequences of Table 1 to germline sequences available from, for example, public antibody sequence databases. The bispecific antigen-binding molecules useful herein, or the antigen-binding domains thereof (D1 and/or D2), which are derived from any of the amino acid sequences shown in Tables 1 and 2, can comprise one or more amino acids within one or more framework and/or CDR regions that are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”).

A person of ordinary skill in the art, starting with the heavy and light chain variable region sequences of Tables 1 and 2, can easily produce numerous bispecific antigen-binding molecules, or antigen-binding domains thereof (D1 and/or D2), which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

In some embodiments, the binding protein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. For example, bispecific antigen-binding molecules, or the antigen-binding domains thereof (D1 and/or D2), that contain one or more germline mutations can be easily tested for one or more desired property such as, improved binding specificity, increased binding affinity, improved or enhanced antagonistic or agonistic biological properties (as the case may be), reduced immunogenicity, etc. Bispecific antigen-binding molecules, or the antigen-binding domains thereof (D1 and/or D2), obtained in this general manner are encompassed within the present disclosure.

In some embodiments, the binding protein is an anti-MET antibody or bispecific antigen-binding molecule comprising variants of any of the HCVR, LCVR, and/or CDR amino acid sequences provided in Tables 1 and 2. Exemplary variants included within this aspect include variants of any of the HCVR, LCVR, and/or CDR amino acid sequences of Tables 1 and 2 having one or more conservative substitutions. For example, binding proteins useful herein include anti-MET antibodies and MET×MET bispecific antigen-binding molecules having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

Exemplary variants also include variants having substantial sequence identity to any of the HCVR, LCVR, and/or CDR amino acid sequences of the antibodies provided in Table 1. As used herein in the context of amino acid sequences, the term “substantial identity” or “substantially identical” means that two amino acid sequences, when optimally aligned, such as by the programs GAP or BESTFIT using default gap weights, share at least 95%, 98% or 99% sequence identity. In certain embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

Sequence identity between two different amino acid sequences is typically measured using sequence analysis software. Sequence analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence provided herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

In some embodiments, the binding protein is an anti-MET antibody or MET×MET bispecific antigen binding protein comprising an Fc domain comprising one or more mutations which enhance or diminish antibody binding to the FcRn receptor, e.g., at acidic pH as compared to neutral pH. For example, anti-MET antibodies and MET×MET bispecific antigen binding proteins can comprise a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the binding protein can be anti-MET antibodies and MET×MET bispecific antigen binding proteins comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains useful herein, are contemplated within the scope of the present disclosure.

In some embodiments, the binding proteins are antibodies and antigen-binding fragments thereof that cross-compete for binding to MET with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

### Binding Protein Characteristics

In some embodiments, the binding protein is an isolated antibody or antigen-binding fragment that binds monomeric human MET with high affinity. For example, binding proteins useful herein include anti-MET antibodies that bind monomeric human MET (e.g., hMET.mmh) with a K_{D} of less than about 230 nM as measured by surface plasmon resonance at 25° C. or 37° C., e.g., using an assay format as defined in Example 3 of U.S. Patent Publication No. 2018-0134794, or a substantially similar assay. According to certain embodiments, anti-MET antibodies useful herein bind monomeric human MET at 37° C. with a K_{D} of less than about 230 nM, less than about 200 nM, less than about 150 nM, less than about 100 nM, less than about 50 nM, less than about 25 nM, less than about 20 nM, less than about 10 nM, less than about 8 nM, less than about 6 nM, less than about 5 nM, less than about 4 nM, or less than about 3 nM, as measured by surface plasmon resonance, e.g., using an assay format as defined in Example 3 of U.S. Patent Publication No. 2018-0134794, or a substantially similar assay.

In some embodiments, the binding protein is an antibody or antigen-binding fragment thereof that binds monomeric human MET (e.g., hMET.mmh) with a dissociative half-life (t1/2) of greater than about 1 minute as measured by surface plasmon resonance at 25° C. or 37° C., e.g., using an assay format as defined in Example 3 of U.S. Patent Publication No. 2018-0134794, or a substantially similar assay. According to certain embodiments, such anti-MET antibodies bind monomeric human MET at 37° C. with a t1/2 of greater than about 1 minute, greater than about 2 minutes, greater than about 4 minutes, greater than about 6 minutes, greater than about 8 minutes, greater than about 10 minutes, greater than about 12 minutes, greater than about 14 minutes, greater than about 16 minutes, greater than about 18 minutes, or greater than about 20 minutes, or longer, as measured by surface plasmon resonance, e.g., using an assay format as defined in Example 3 of U.S. Patent Publication No. 2018-0134794, or a substantially similar assay.

In some embodiments, the binding protein is an antibody or antigen-binding fragment thereof that binds dimeric human MET (e.g., hMET.mFc) with high affinity. For example, such anti-MET antibodies bind dimeric human MET with a K_{D} of less than about 3 nM as measured by surface plasmon resonance at 25° C. or 37° C., e.g., using an assay format as defined in Example 3 of U.S. Patent Publication No. 2018-0134794, or a substantially similar assay. Anti-MET antibodies useful herein can bind dimeric human MET at 37° C. with a K_{D} of less than about 3 nM, less than about 2 nM, less than about 1 nM, less than about 0.9 nM, less than about 0.8 nM, less than about 0.7 nM, less than about 0.6 nM, less than about 0.5 nM, less than about 0.4 nM, less than about 0.3 nM, or less than about 0.25 nM, as measured by surface plasmon resonance, e.g., using an assay format as defined in Example 3 of U.S. Patent Publication No. 2018-0134794, or a substantially similar assay.

In some embodiments, the binding protein is an antibody or antigen-binding fragment thereof that binds dimeric human MET (e.g., hMET.mFc) with a dissociative half-life (t1/2) of greater than about 4 minutes as measured by surface plasmon resonance at 25° C. or 37° C., e.g., using an assay format as defined in Example 3 of U.S. Patent Publication No. 2018-0134794, or a substantially similar assay. According to certain embodiments, anti-MET antibodies useful herein bind dimeric human MET at 37° C. with a t1/2 of greater than about 4 minutes, greater than about 5 minutes, greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 105 minutes, or longer, as measured by surface plasmon resonance, e.g., using an assay format as defined in Example 3 of U.S. Patent Publication No. 2018-0134794, or a substantially similar assay.

In some embodiments, the binding protein is an antibody or antigen-binding fragment thereof that binds dimeric human MET (e.g., hMET.mFc) with a dissociative half-life (t1/2) of greater than about 10 minutes as measured by surface plasmon resonance at 25° C. or 37° C., e.g., using an assay format as defined in Example 5 of U.S. Patent Publication No. 2018-0134794, or a substantially similar assay. According to certain embodiments, a MET×MET bispecific antigen-binding protein useful herein binds dimeric human MET at 37° C. with a t1/2 of greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, greater than about 1100 minutes, or longer, as measured by surface plasmon resonance, e.g., using an assay format as defined in Example 5 of U.S. Patent Publication No. 2018-0134794, or a substantially similar assay.

In some embodiments, the binding protein is an antibody or antigen-binding fragment thereof, for example, a MET×MET bispecific antigen-binding protein, that blocks the interaction between HGF and MET, e.g., in an in vitro ligand-binding assay. A MET×MET bispecific antigen-binding protein useful herein can block HGF binding to cells expressing human MET, and induce minimal or no MET activation in the absence of HGF signaling. For example, useful herein are MET×MET bispecific antigen-binding proteins that exhibit a degree of MET agonist activity in a cell-based MET activity reporter assay that is less than 50%, less than 40%, less than 30%, less than 20%, less than 10%, less than 5%, less than 3%, less than 2% or less than 1% of the MET agonist activity observed in an equivalent activity reporter assay using a monospecific antibody comprising D1 or D2 alone.

In one embodiment, the antibody or fragment thereof is a human monoclonal antibody or antigen-binding fragment thereof that binds to MET, wherein the antibody or fragment thereof exhibits one or more of the following characteristics: (i) comprises a HCVR having an amino acid sequence selected from the group consisting of SEQ ID NO: 2, 10, 18, 26, 34, 42, 50, 58, 66, 74, 82, 90, 98, 106, 114, 122, and 130, or a substantially similar sequence thereof having at least 90%, at least 95%, at least 98% or at least 99% sequence identity; (ii) comprises a LCVR having an amino acid sequence of SEQ ID NO: 138, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 16, 24, 32, 40, 48, 56, 64, 72, 80, 88, 96, 104, 112, 120, 128 and 136, or a substantially similar sequence thereof having at least 90%, at least 95%, at least 98% or at least 99% sequence identity; and a LCDR3 domain having an amino acid sequence of SEQ ID NO: 144, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 12, 20, 28, 36, 44, 52, 60, 68, 76, 84, 92, 100, 108, 116, 124, and 132, or a substantially similar sequence thereof having at least 90%, at least 95%, at least 98% or at least 99% sequence identity; a HCDR2 domain having an amino acid sequence selected from the group consisting of SEQ ID NO: 6, 14, 22, 30, 38, 46, 54, 62, 70, 78, 86, 94, 102, 110, 118, 126, and 134, or a substantially similar sequence thereof having at least 90%, at least 95%, at least 98% or at least 99% sequence identity; a LCDR1 domain having an amino acid sequence of SEQ ID NO: 140, or a substantially similar sequence thereof having at least 90%, at least 95%, at least 98% or at least 99% sequence identity; and a LCDR2 domain having an amino acid sequence of SEQ ID NO: 142, or a substantially similar sequence thereof having at least 90%, at least 95%, at least 98% or at least 99% sequence identity; (v) is a multi-specific antigen-binding molecule comprising a first binding specificity to MET and a second binding specificity to a tumor specific antigen; (vi) is a multi-specific antigen-binding molecule comprising a first binding specificity to one epitope of MET and a second binding specificity to a second epitope of MET; (vii) binds to monomeric human MET (e.g., hMET.mmh) with a K_{D} of less than about 230 nM as measured by surface plasmon resonance at 25° C. or 37° C.; (viii) binds to dimeric human MET with a K_{D} of less than about 3 nM as measured by surface plasmon resonance at 25° C. or 37° C.; (ix) blocks the binding of HGF to MET; and (x) suppresses tumor growth and increases survival in subjects with cancer.

In one embodiment, the antibody or fragment thereof is a human monoclonal antibody or antigen-binding fragment thereof that blocks HGF binding to MET, wherein the antibody or fragment thereof exhibits one or more of the following characteristics: (i) comprises a HCVR having an amino acid sequence selected from the group consisting of SEQ ID NO: 2, 10, 18, 26, 34, 42, 50, 58, 66, 74, 82, 90, 98, 106, 114, 122, and 130, or a substantially similar sequence thereof having at least 90%, at least 95%, at least 98% or at least 99% sequence identity; (ii) comprises a LCVR having an amino acid sequence of SEQ ID NO: 138, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 16, 24, 32, 40, 48, 56, 64, 72, 80, 88, 96, 104, 112, 120, 128 and 136, or a substantially similar sequence thereof having at least 90%, at least 95%, at least 98% or at least 99% sequence identity; and a LCDR3 domain having an amino acid sequence of SEQ ID NO: 144, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 12, 20, 28, 36, 44, 52, 60, 68, 76, 84, 92, 100, 108, 116, 124, and 132, or a substantially similar sequence thereof having at least 90%, at least 95%, at least 98% or at least 99% sequence identity; a HCDR2 domain having an amino acid sequence selected from the group consisting of SEQ ID NO: 6, 14, 22, 30, 38, 46, 54, 62, 70, 78, 86, 94, 102, 110, 118, 126, and 134, or a substantially similar sequence thereof having at least 90%, at least 95%, at least 98% or at least 99% sequence identity; a LCDR1 domain having an amino acid sequence of SEQ ID NO: 140, or a substantially similar sequence thereof having at least 90%, at least 95%, at least 98% or at least 99% sequence identity; and a LCDR2 domain having an amino acid sequence of SEQ ID NO: 142, or a substantially similar sequence thereof having at least 90%, at least 95%, at least 98% or at least 99% sequence identity; (v) is a multi-specific antigen-binding molecule comprising a first binding specificity to MET and a second binding specificity to a tumor specific antigen; (vi) is a multi-specific antigen-binding molecule comprising a first binding specificity to one epitope of MET and a second binding specificity to a second epitope of MET; (vii) binds to monomeric human MET (e.g., hMET.mmh) with a K_{D} of less than about 230 nM as measured by surface plasmon resonance at 25° C. or 37° C.; (viii) binds to dimeric human MET with a K_{D} of less than about 3 nM as measured by surface plasmon resonance at 25° C. or 37° C.; and (ix) suppresses tumor growth and increases survival in subjects with cancer.

In certain embodiments, the binding protein is a MET×MET bispecific antibody or antigen-binding fragment thereof, wherein a first antigen-binding domain (D1) binds a first epitope of human MET and a second antigen-binding domain (D2) binds a second epitope of human MET, either in natural form, or recombinantly produced, or to a fragment thereof. In some aspects, D1 and D2 do not compete with one another for binding to human MET. In some embodiments, the binding protein exhibits minimal agonist activity in a cell-based MET activity reporter assay. In some embodiments, the bispecific antigen-binding molecule exhibits a degree of MET agonist activity in a cell-based MET activity reporter assay that is less than 10% of the MET agonist activity of a monovalent antigen-binding molecule comprising D1 or D2 alone. In some embodiments, the bispecific antigen-binding molecule promotes degradation of cell surface-expressed MET. In some embodiments, the bispecific antigen-binding molecule inhibits the growth or promotes tumor regression of tumors harboring MET genetic alterations. In some embodiments, the bispecific antigen-binding molecule inhibits the growth or promotes tumor regression of tumors whose growth is driven by autocrine HGF signaling.

In some embodiments, anti-MET antibodies or MET×MET bispecific antibodies useful herein bind to the same epitope, or a portion of the epitope, as any of the specific exemplary antibodies described herein in Table 1 or Table 2, or an antibody having the CDR sequences of any of the exemplary antibodies described in Table 1 or Table 2. Likewise, suitable binding proteins also include anti-MET antibodies or MET×MET bispecific antibodies that compete for binding to MET or a MET fragment with any of the specific exemplary antibodies described herein in Table 1 or Table 2, or an antibody having the CDR sequences of any of the exemplary antibodies described in Table 1 or Table 2. For example, suitable binding proteins include anti-MET antibodies and MET×MET bispecific antibodies that cross-compete for binding to MET with one or more antibodies as defined in U.S. Patent Publication No. 2018-0134794, or cross-compete for binding to MET with one or more antibodies as defined in U.S. Patent Publication No. 2018-0134794.

The antibodies and antigen-binding fragments described herein specifically bind to MET and modulate the interaction of MET with HGF. The MET×MET bispecific antibodies may bind to MET with high affinity or with low affinity. In certain embodiments, the antibodies are blocking antibodies wherein the antibodies bind to MET and block the interaction of MET with HGF. In some embodiments, the blocking antibodies of the disclosure block the binding of HGF to MET. In some embodiments, the blocking antibodies are useful for treating a subject suffering from cancer. They may be used to inhibit the growth of tumor cells in a subject. They may be used alone or as adjunct therapy with other therapeutic moieties or modalities known in the art for treating cancer. In certain embodiments, the MET×MET bispecific antibodies that bind to MET with a low affinity are used as multi-specific antigen-binding molecules wherein the first binding specificity binds to MET with a low affinity and the second binding specificity binds to a different epitope of MET or a tumor specific antigen.

Certain anti-MET antibodies and MET×MET bispecific antibodies of the present disclosure are able to bind to and neutralize the activity of MET, as determined by in vitro or in vivo assays. The ability of the antibodies of the disclosure to bind to and neutralize the activity of MET may be measured using any standard method known to those skilled in the art, including binding assays, or activity assays, as described herein.

Non-limiting, exemplary in vitro assays for measuring binding activity are illustrated in Examples 3 and 6 of US-2018-0134794 A1. In Example 6, the binding affinities and kinetic constants of human MET×MET bispecific antibodies for human MET were determined by surface plasmon resonance and the measurements were conducted on a T200 Biacore instrument. In Example 7 US-2018-0134794, blocking assays were used to determine the ability of the anti-MET antibodies and MET×MET bispecific antibodies to block MET-binding ability of HGF. In Example 4 of US-2018-0134794, blocking assays were used to determine cross-competition between different anti-MET antibodies. Example 8 of US-2018-0134794 describes the growth inhibition of cells overexpressing MET by anti-MET antibodies and MET×MET bispecific antibodies. In Example 10 of US-2018-0134794, a MET×MET bispecific antibody is shown to induce MET degradation and inhibit both MET and ERK phosphorylation. US-2018-0134794 also provides several examples demonstrating tumor growth inhibition or tumor regression, both in vivo and in vitro, induced by a MET×MET bispecific antibody.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to MET. An antibody fragment may include a Fab fragment, a F(ab′)2 fragment, a Fv fragment, a dAb fragment, a fragment containing a CDR, or an isolated CDR. In certain embodiments, the term “antigen-binding fragment” refers to a polypeptide or fragment thereof of a multi-specific antigen-binding molecule. In such embodiments, the term “antigen-binding fragment” includes, e.g., an extracellular domain of HGF which binds specifically to MET. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a Vu domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

The anti-MET antibodies and MET×MET bispecific antibodies and antibody fragments useful herein encompass proteins having amino acid sequences that vary from those of the described antibodies, but that retain the ability to bind MET. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a subject can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

Anti-MET antibodies and MET×MET bispecific antibodies useful herein can comprise an Fc domain comprising one or more mutations which enhance or diminish antibody binding to the FcRn receptor, e.g., at acidic pH as compared to neutral pH. For example, the present disclosure includes MET×MET bispecific antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, useful herein are anti-MET antibodies and MET×MET bispecific antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P2571 and Q3111); 2571 and 434H (e.g., P2571 and N434H); 376V and 434H (e.g., D376V and N434H); 307A, 380A and 434A (e.g., T307A, E380A and N434A); and 433K and 434F (e.g., H433K and N434F). In one embodiment, the present disclosure includes MET×MET bispecific antibodies comprising an Fc domain comprising a S108P mutation in the hinge region of IgG4 to promote dimer stabilization. All possible combinations of the foregoing Fc domain mutations, and other mutations within the antibody variable domains provided herein in Table 1, are contemplated within the scope of the present disclosure.

Anti-MET antibodies and MET×MET bispecific antibodies useful herein can comprise a chimeric heavy chain constant (CH) region, wherein the chimeric CH region comprises segments derived from the CH regions of more than one immunoglobulin isotype. For example, the antibodies may comprise a chimeric CH region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies may comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., USSN. 14/170,166, filed Jan. 31, 2014, the disclosure of which is hereby incorporated by reference in its entirety).

### B. Positron Emitters and Chelating Moieties

Suitable positron emitters include, but are not limited to, those that form stable complexes with the chelating moiety and have physical half-lives suitable for immuno-PET imaging purposes. Illustrative positron emitters include, but are not limited to, ⁸⁹Zr, ⁶⁸Ga, ⁶⁴ Cu, ⁴⁴Sc, and ⁸⁶Y. Suitable positron emitters also include those that directly bond with the MET binding protein, including, but not limited to, ⁷⁶Br and ¹²⁴I, and those that are introduced via prosthetic group, e.g., ¹⁸F,

The chelating moieties described herein are chemical moieties that are covalently linked to the MET binding protein, e.g., MET×MET bispecific antibody, and comprise a portion capable of chelating a positron emitter, i.e., capable of reacting with a positron emitter to form a coordinated chelate complex. Suitable moieties include those that allow efficient loading of the particular metal and form metal-chelator complexes that are sufficiently stable in vivo for diagnostic uses, e.g., immuno-PET imaging. Illustrative chelating moieties include those that minimize dissociation of the positron emitter and accumulation in mineral bone, plasma proteins, and/or bone marrow depositing to an extent suitable for diagnostic uses.

Examples of chelating moieties include, but are not limited to, those that form stable complexes with positron emitters ⁸⁹Zr, ⁶⁸Ga, ⁶⁴Cu, ⁴⁴Sc, and/or ⁸⁶Y. Illustrative chelating moieties include, but are not limited to, those described in Nature Protocols, 5 (4): 739, 2010; Bioconjugate Chem., 26 (12): 2579 (2015); Chem Commun (Camb), 51 (12): 2301 (2015); Mol. Pharmaceutics, 12:2142 (2015); Mol. Imaging Biol., 18:344 (2015); Eur. J. Nucl. Med. Mol. Imaging, 37:250 (2010); Eur. J. Nucl. Med. Mol. Imaging (2016). doi: 10.1007/s00259-016-3499-x; Bioconjugate Chem., 26 (12): 2579 (2015); WO 2015/140212A1; and U.S. Pat. No. 5,639,879, incorporated by reference in their entireties.

Illustrative chelating moieties also include, but are not limited to, those that comprise desferrioxamine (DFO) (also known as deferoxamine), 1,4,7,10-tetraacetic acid (DOTA), diethylenetriaminepentaacetic acid (DTPA), ethylenediaminetetraacetic acid (EDTA), (1,4,7,10-Tetraazacyclododecane-1,4,7,10-tetra(methylene phosphonic) acid (DOTP), 1R, 4R, 7R, 10R)-a′a″a′″-Tetramethyl-1,4,7,10-tetraazacyclododecane-1,4,7,10-tetraacetic acid (DOTMA), 1,4,8,11-Tetraazacyclotetradecane-1,4,8,11-tetraacetic acid (TETA), H₄octapa, Hophospa, Hadedpa, Hsdecapa, H₂azapa, HOPO, DO2A, 1,4,7,10-Tetrakis(carbamoylmethyl)-1,4,7,10-tetraazacyclododecane (DOTAM), 1,4,7-triazacyclononane-N,N′, N″-triacetic acid (NOTA), 1,4,7,10-Tetrakis(carbamoylmethyl)-1,4,7,10-tetraazacyclododecane (DOTAM), 1,4,8,11-tetraazabicyclo[6.6.2]hexadecane-4,11-dicetic acid (CB-TE2A), 1,4,7,10-Tetraazacyclododecane (Cyclen), 1,4,8,11-Tetraazacyclotetradecane (Cyclam), octadentate chelators, hexadentate chelators, phosphonate-based chelators, macrocyclic chelators, chelators comprising macrocyclic terephthalamide ligands, bifunctional chelators, fusarinine C and fusarinine C derivative chelators, triacetylfusarinine C (TAFC), ferrioxamine E (FOXE), ferrioxamine B (FOXB), ferrichrome A (FCHA), and the like.

In some embodiments, the chelating moieties are covalently bonded to the MET binding protein, e.g., antibody or antigen binding fragment thereof, via a linker moiety, which covalently attaches the chelating portion of the chelating moiety to the binding protein. In some embodiments, these linker moieties are formed from a reaction between a reactive moiety of the MET binding protein, e.g., cysteine or lysine of an antibody, and reactive moiety that is attached to a chelator, including, for example, a p-isothiocyanatobenyl group and the reactive moieties provided in the conjugation methods below. In addition, such linker moieties optionally comprise chemical groups used for purposes of adjusting polarity, solubility, steric interactions, rigidity, and/or the length between the chelating portion and MET binding protein.

### C. Preparation of Radiolabeled MET Binding Protein Conjugates

The radiolabeled anti-MET antibody or MET×MET bispecific antibody conjugates can be prepared by (1) reacting a MET binding protein, e.g., a MET×MET bispecific antibody, with a molecule comprising a positron emitter chelator and a moiety reactive to the desirable conjugation site of the MET binding protein and (2) loading the desirable positron emitter.

Suitable conjugation sites include, but are not limited to, lysine and cysteine, both of which can be, for example, native or engineered, and can be, for example, present on the heavy or light chain of an antibody. Cysteine conjugation sites include, but are not limited to, those obtained from mutation, insertion, or reduction of antibody disulfide bonds. Methods for making cysteine engineered antibodies include, but are not limited to, those disclosed in WO2011/056983. Site-specific conjugation methods can also be used to direct the conjugation reaction to specific sites of an antibody, achieve desirable stoichiometry, and/or achieve desirable drug-to-antibody (DAR) ratios. Such conjugation methods are known to those of ordinary skill in the art and include, but are not limited to cysteine engineering and enzymatic and chemo-enzymatic methods, including, but not limited to, glutamine conjugation, Q295 conjugation, and transglutaminase-mediated conjugation, as well as those described in J. Clin. Immunol., 36:100 (2016), incorporated herein by reference in its entirety. Suitable moieties reactive to the desirable conjugation site generally enable efficient and facile coupling of the MET binding protein, e.g., antibody and positron emitter chelator. Moieties reactive to lysine and cysteine sites include electrophilic groups, which are known to those of ordinary skill. In certain aspects, when the desired conjugation site is lysine, the reactive moiety is an isothiocyanate, e.g., p-isothiocyanatobenyl group or reactive ester. In certain aspects, when the desired conjugation site is cysteine, the reactive moiety is a maleimide.

When the chelator is desferrioxamine (DFO) (also known as deferoxamine), suitable reactive moieties include, but are not limited to, an isothiocyantatobenzyl group, an n-hydroxysuccinimide ester,2,3,5,6 tetraflurorphenol ester, n-succinimidyl-S-acetylthioacetate, and those described in BioMed Research International, Vol 2014, Article ID 203601, incorporated herein by reference in its entirety. In certain embodiments, the MET binding protein is an antibody and the molecule comprising a positron emitter chelator and moiety reactive to the conjugation site is p-isothiocyantatobenzyl-desferrioxamine (p-SCN-Bn-DFO):

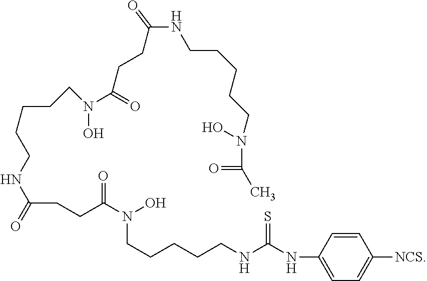

Positron emitter loading is accomplished by incubating the MET binding protein chelator conjugate with the positron emitter for a time sufficient to allow coordination of said positron emitter to the chelator, e.g., by performing the methods described in the examples provided herein, or substantially similar method.

### D. Illustrative Embodiments of Conjugates

Included in the instant disclosure are radiolabeled antibody conjugates comprising an antibody or antigen binding fragment thereof that binds human MET, e.g. an anti-MET antibody or a MET×MET bispecific antibody, and a positron emitter. Also included in the instant disclosure are radiolabeled antibody conjugates comprising an anti-MET antibody or a MET×MET bispecific antibody, a chelating moiety, and a positron emitter.

In some embodiments, the chelating moiety comprises a chelator capable of forming a complex with ⁸⁹Zr. In certain embodiments, the chelating moiety comprises desferrioxamine. In certain embodiments, the chelating moiety is p-isothiocyanatobenzyl-desferrioxamine.

In some embodiments, the positron emitter is ⁸⁹Zr. In some embodiments, less than 1.0% of the MET binding protein is conjugated with the positron emitter, less than 0.9% of the MET binding protein is conjugated with the positron emitter, less than 0.8% of the MET binding protein is conjugated with the positron emitter, less than 0.7% of the MET binding protein is conjugated with the positron emitter, less than 0.6% of the MET binding protein is conjugated with the positron emitter, less than 0.5% of the MET binding protein is conjugated with the positron emitter, less than 0.4% of the MET binding protein is conjugated with the positron emitter, less than 0.3% of the MET binding protein is conjugated with the positron emitter, less than 0.2% of the MET binding protein is conjugated with the positron emitter, or less than 0.1% of the MET binding protein is conjugated with the positron emitter.

In some embodiments, the chelating moiety-to-antibody ratio of the conjugate is from 1 to 2. As used herein, “chelating moiety-to-antibody ratio” is the average chelator moiety to antibody ratio and is a measure of chelator load per antibody. This ratio is analogous to “DAR”, i.e., drug-antibody ratio, which is used by those skilled in the art to measure drug load per antibody for antibody-drug conjugates (ADCs); in the context of the conjugates described herein for iPET imaging, the chelating moiety-to-antibody ratio can be ascertained using methods described herein and others known in the art for the determination of DAR, e.g. those described in Wang et al., Antibody-Drug Conjugates, The 21st Century Magic Bullets for Cancer (2015). In some embodiments, the chelating moiety-to-antibody ratio is from 1.0 to 4.0, or about 1.0 to 3.0, or about 1.0 to 2.0. In some embodiments, the chelating moiety-to-antibody ratio is about 1.26, for example, about 1.3.

In a particular embodiment, chelating moiety is p-isothiocyanatobenzyl-desferrioxamine and the positron emitter is ⁸⁹Zr. In another particular embodiment, the chelating moiety is p-isothiocyanatobenzyl-desferrioxamine and the positron emitter is ⁸⁹Zr, and the chelating moiety-to-antibody ratio of the conjugate is from 1 to 2.

In some embodiments, provided herein are antigen-binding proteins that bind MET, wherein said antigen-binding proteins that bind MET are covalently bonded to one or more moieties having the following structure:

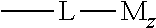

wherein L is a chelating moiety; M is a positron emitter; and z, independently at each occurrence, is 0 or 1; and wherein at least one of z is 1. In certain embodiments, the radiolabeled antigen-binding protein is a compound of Formula (I):

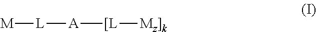

A is a protein that binds MET; L is a chelating moiety; M is a positron emitter; z is 0 or 1; and k is an integer from 0-30. In some embodiments, k is 1. In some embodiments, k is 2.

In some embodiments, L is:

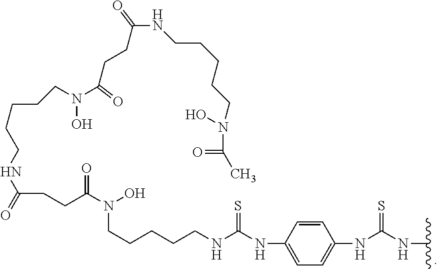 [SMILES: *NC(=S)Nc1ccc(NC(=S)NCCCCCN(O)C(=O)CCC(=O)NCCCCCN(O)C(=O)CCC(=O)NCCCCCN(O)C(C)=O)cc1]

In some embodiments, M is ⁸⁹Zr.

In some embodiments, k is an integer from 1 to 2. In some embodiments, k is 1. In some embodiments, k is 2.

In some embodiments, —L-M is

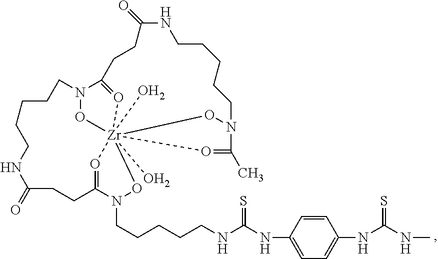

Included in the instant disclosure are also methods of synthesizing a radiolabeled antibody conjugates comprising contacting a compound of Formula (III):

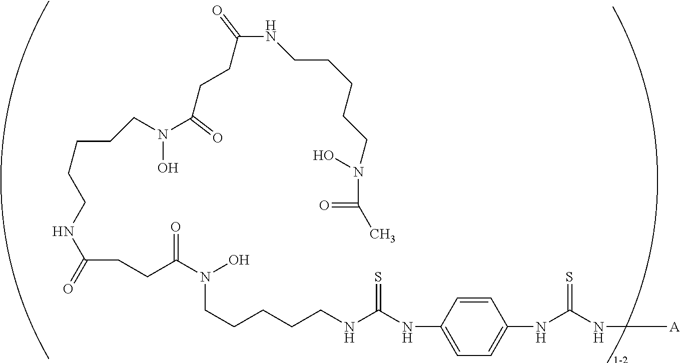

with ⁸⁹Zr, wherein A is an antibody or antigen-binding fragment thereof that binds MET. In certain embodiments, the compound of Formula (III) is synthesized by contacting an antibody, or antigen binding fragment thereof, that binds MET, with p-SCN-Bn-DFO.

Provided herein is also the product of the reaction between a compound of Formula (III) with ⁸⁹Zr.

Provided herein are compounds of Formula (III):

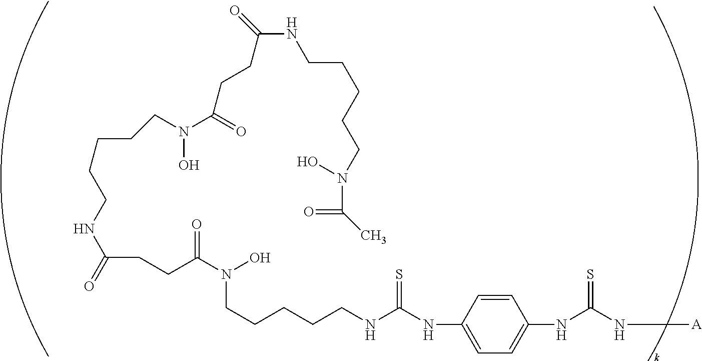

wherein A is an antibody or antigen binding fragment thereof that binds MET and k is an integer from 1-30. In some embodiments, k is 1 or 2.

Provided herein are antibody conjugates comprising (i) an antibody or antigen-binding fragment thereof that binds MET and (ii) one or more chelating moieties. In some embodiments, the chelating moiety comprises:

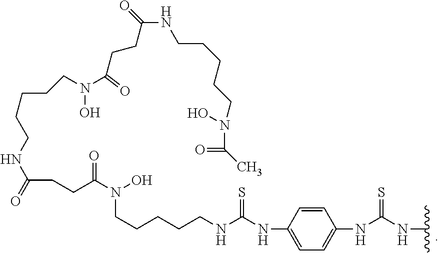 [SMILES: *NC(=S)Nc1ccc(NC(=S)NCCCCCN(O)C(=O)CCC(=O)NCCCCCN(O)C(=O)CCC(=O)NCCCCCN(O)C(C)=O)cc1]

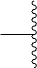

is a covalent bond to the antibody or antigen-binding fragment thereof.

In some aspects, the antibody conjugate has a chelating moiety-to-antibody ratio of from about 1.0 to about 2.0. In some aspects, the antibody conjugate has a chelating moiety-to-antibody ratio of about 1.3.

In some embodiments, provided herein are compositions comprising a conjugate having the following structure:

A-Lₖ

wherein A is a protein that binds MET; L is a chelating moiety; and k is an integer from 1-30; wherein the conjugate is chelated with a positron emitter in an amount sufficient to provide a specific activity suitable for clinical PET imaging. In some embodiments, the amount of chelated positron emitter is an amount sufficient to provide a specific activity of about 1 to about 50 mCi per 1-50 mg of the protein that binds MET.

In some embodiments, the amount of chelated positron emitter is an amount sufficient to provide a specific activity of up to 25 mCi, up to 20 mCi, up to 15 mCi, up to 12 mCi, or up to 10 mCi per 1-50 mg of the protein that binds MET, for example, in a range of about 3 to about 25 mCi, about 10 to about 25 mCi, about 1 to about 15 mCi, about 3 to about 15 mCi, about 5 to about 25 mCi, about 15 to about 25 mCi, or about 3 to about 10 mCi, or about 12 mCi, or about 21 mCi.

In some embodiments, the antibody or antigen-binding fragment thereof binds monomeric human MET with a binding dissociation equilibrium constant (K_{D}) of less than about 230 nM as measured in a surface plasmon resonance assay at 25° C. or 37° C.

In some embodiments, the antibody or antigen-binding fragment thereof binds dimeric human MET with a K_{D} less than about 3 nM in a surface plasmon resonance assay at 25° C. or 37° C.

In some embodiments, the antibody or antigen-binding fragment thereof competes for binding to human MET with a reference antibody comprising the complementarity determining regions (CDRs) of a HCVR, wherein the HCVR has an amino acid sequence selected from the group consisting of HCVR sequences listed in Table 1; and the CDRs of a LCVR, wherein the LCVR has an amino acid sequence shown in Table 1. In some embodiments, the reference antibody or antigen-binding fragment thereof comprises an HCVR/LCVR amino acid sequence pair as set forth in Table 1. In some embodiments, the reference antibody comprises an HCVR/LCVR amino acid sequence pair selected from the group consisting of SEQ ID NOs: 2/138, 10/138, 18/138, 26/138, 34/138, 42/138, 50/138, 58/138, 66/138, 74/138, 82/138, 90/138, 98/138,106/138, 114/138, 122/138 and 130/138.

In some embodiments, the antibody or antigen binding fragment thereof blocks HGF binding to MET. In some embodiments, the antibody or antigen binding fragment thereof do not increase or decrease MET binding to its ligands.

In some embodiments, the antibody or antigen-binding fragment thereof comprises the complementarity determining regions (CDRs) of a HCVR, wherein the HCVR has an amino acid sequence selected from the group consisting of SEQ ID NOs: 2, 10, 18, 26, 34, 42, 50, 58, 66, 74, 82, 90, 98, 106, 114, 122 and 130; and the CDRs of a LCVR, wherein the LCVR has an amino acid sequence of SEQ ID NO: 138. In certain embodiments, the isolated antibody comprises an HCVR/LCVR amino acid sequence pair selected from the group consisting of SEQ ID NOs: 2/138, 10/138, 18/138, 26/138, 34/138, 42/138, 50/138, 58/138, 66/138, 74/138, 82/138, 90/138, 98/138,106/138, 114/138, 122/138 and 130/138.

In some embodiments, the antibody is a human monoclonal antibody or antigen-binding fragment thereof that binds specifically to human MET, wherein the antibody or antigen-binding fragment thereof comprises a heavy chain variable region (HCVR) having an amino acid sequence selected from the group consisting of HCVR sequences listed in Table 1.

In some embodiments, the antibody is a human monoclonal antibody or antigen-binding fragment thereof that binds specifically to human MET, wherein the antibody or antigen-binding fragment thereof comprises a light chain variable region (LCVR) having an amino acid sequence as shown in Table 1.

In some embodiments, the antibody a human monoclonal antibody or antigen-binding fragment thereof that binds specifically to human MET, wherein the antibody or antigen-binding fragment thereof comprises (a) a HCVR having an amino acid sequence selected from the group consisting of HCVR sequences listed in Table 1; and (b) a LCVR having an amino acid sequence as shown in Table 1.

In some embodiments, the antibody or antigen-binding fragment thereof comprises three heavy chain complementarity determining regions (CDRs) (HCDR1, HCDR2 and HCDR3) contained within any one of the heavy chain variable region (HCVR) sequences listed in Table 1; and three light chain CDRs (LCDR1, LCDR2 and LCDR3) contained within the light chain variable region (LCVR) sequence shown in Table 1.

In some embodiments, the antibody or antigen-binding fragment thereof comprises:
(a) a HCDR1 domain having an amino acid sequence selected from the group consisting of SEQ ID NOs: 4, 12, 20, 28, 36, 44, 52, 60, 68, 76, 84, 92, 100, 108, 116, 124 and 132;
(b) a HCDR2 domain having an amino acid sequence selected from the group consisting of SEQ ID NOs: 6, 14, 22, 30, 38, 46, 54, 62, 70, 78, 86, 94, 102, 110, 118, 126, and 134;
(c) a HCDR3 domain having an amino acid sequence selected from the group consisting of SEQ ID NOs: 8, 16, 24, 32, 40, 48, 56, 64, 72, 80, 88, 96, 104, 112, 120, 128 and 136;
(d) a LCDR1 domain having an amino acid sequence of SEQ ID NO: 140;
(e) a LCDR2 domain having an amino acid sequence of SEQ ID NO: 142; and
(f) a LCDR3 domain having an amino acid sequence of SEQ ID NO: 144.

In some embodiments, the antibody or antigen-binding fragment comprises a HCVR/LCVR amino acid sequence pair selected from the group consisting of SEQ ID NOS: 2/138, 10/138, 18/138, 26/138, 34/138, 42/138, 50/138, 58/138, 66/138, 74/138, 82/138, 90/138, 98/138,106/138, 114/138, 122/138, and 130/138.

In some embodiments, the antibody or antigen-binding fragment thereof comprises the CDRs of a HCVR, wherein the HCVR has an amino acid sequence selected from the group consisting of SEQ ID NOs: 2, 10, 18, 26, 34, 42, 50, 58, 66, 74, 82, 90, 98, 106, 114, 122 and 130; and the CDRs of a LCVR, wherein the LCVR has an amino acid sequence of SEQ ID NO: 138.

In some embodiments, the antibody or antigen-binding fragment thereof is an anti-MET antibody comprising the CDRs within the HCVR/LCVR amino acid sequence pair of SEQ ID NOs: 82/138. In some embodiments, antibody or antigen-binding fragment thereof is an anti-MET antibody comprising the HCVR amino acid sequence of SEQ ID NO: 82 and the LCVR amino acid sequence of SEQ ID NO: 138.

In some embodiments, the antibody or antigen-binding fragment thereof is an anti-MET antibody comprising the CDRs within the HCVR/LCVR amino acid sequence pair of SEQ ID NOs: 18/138. In some embodiments, antibody or antigen-binding fragment thereof is an anti-MET antibody comprising the HCVR amino acid sequence of SEQ ID NO: 18 and the LCVR amino acid sequence of SEQ ID NO: 138.

In some embodiments, the antibody or antigen-binding fragment thereof is an anti-MET antibody comprising the CDRs within the HCVR/LCVR amino acid sequence pair of SEQ ID NOs: 58/138. In some embodiments, antibody or antigen-binding fragment thereof is an anti-MET antibody comprising the HCVR amino acid sequence of SEQ ID NO: 58 and the LCVR amino acid sequence of SEQ ID NO: 138.

In some embodiments, the antibody or antigen-binding fragment thereof is a MET×MET bispecific antigen-binding protein comprising the CDRs within the D1-HCVR amino acid sequence of SEQ ID NO: 58 and the CDRs within the D2-HCVR amino acid sequence of SEQ ID NO: 82. In some aspects, the MET×MET bispecific antigen-binding protein further comprises the CDRs within the LCVR amino acid sequence of SEQ ID NO: 138. In some embodiments, the antibody or antigen-binding fragment thereof is a MET×MET bispecific antigen-binding protein comprising the D1-HCVR amino acid sequence of SEQ ID NO: 58 and the D2-HCVR amino acid sequence of SEQ ID NO: 82. In some aspects, the MET×MET bispecific antigen-binding protein further comprises the LCVR amino acid sequence of SEQ ID NO: 138.

In some embodiments, the antibody or antigen-binding fragment thereof is a MET×MET bispecific antigen-binding protein comprising the CDRs within the D1-HCVR amino acid sequence of SEQ ID NO: 18 and the CDRs within the D2-HCVR amino acid sequence of SEQ ID NO: 82. In some aspects, the MET×MET bispecific antigen-binding protein further comprises the CDRs within the LCVR amino acid sequence of SEQ ID NO: 138. In some embodiments, the antibody or antigen-binding fragment thereof is a MET×MET bispecific antigen-binding protein comprising the D1-HCVR amino acid sequence of SEQ ID NO: 18 and the D2-HCVR amino acid sequence of SEQ ID NO: 82.

In some embodiments, the radiolabeled antibody conjugate comprises an antibody or antigen binding fragment thereof that binds MET, a chelating moiety, and a positron emitter, wherein the antibody or antigen-binding fragment thereof that binds MET comprises the CDRs within the D1-HCVR amino acid sequence of SEQ ID NO: 58, the CDRs within the D2-HCVR amino acid sequence of SEQ ID NO: 82, and the CDRs within the LCVR amino acid sequence of SEQ ID NO: 138, the chelating moiety is desferrioxamine, and the positron emitter is ⁸⁹Zr.

In some embodiments, the radiolabeled antibody conjugate comprises an antibody or antigen binding fragment thereof that binds MET, a chelating moiety, and a positron emitter, wherein the antibody or antigen-binding fragment thereof that binds MET comprises the D1-HCVR amino acid sequence of SEQ ID NO: 58, the D2-HCVR amino acid sequence of SEQ ID NO: 82, and the LCVR amino acid sequence of SEQ ID NO: 138, the chelating moiety is desferrioxamine, and the positron emitter is ⁸⁹Zr.

### III. Methods of Using Radiolabeled Immunoconjugates

In certain aspects, the present disclosure provides diagnostic and therapeutic methods of use of the radiolabeled antibody conjugates of the present disclosure.

According to one aspect, the present disclosure provides methods of detecting MET in a tissue, the methods comprising administering a radiolabeled antibody conjugate of the provided herein to the tissue; and visualizing the MET expression by positron emission tomography (PET) imaging. In certain embodiments, the tissue comprises cells or cell lines. In certain embodiments, the tissue is present in a subject, wherein the subject is a mammal. In certain embodiments, the subject is a human subject. In certain embodiments, the subject has cancer.

According to one aspect, the present disclosure provides methods of imaging a tissue that expresses MET comprising administering a radiolabeled antibody conjugate of the present disclosure to the tissue; and visualizing the MET expression by positron emission tomography (PET) imaging. In one embodiment, the tissue is comprised in a tumor. In one embodiment, the tissue is comprised in a tumor cell culture or tumor cell line. In one embodiment, the tissue is comprised in a tumor lesion in a subject.

According to one aspect, the present disclosure provides methods for measuring response to an anti-MET therapy in a subject having cancer, wherein the response to therapy is measured by the change in MET expression relative to MET expression prior to therapy. The methods, according to this aspect, comprise administering a radiolabeled antibody conjugate provided herein to a subject in need thereof and visualizing the MET expression by positron emission tomography (PET) imaging. A decrease in MET expression, relative to MET expression prior to therapy, correlates to a positive response to anti-MET therapy.

According to one aspect, the present disclosure provides methods for determining if a subject with a solid tumor is suitable for anti-tumor therapy comprising an inhibitor of the HGF/MET signaling pathway, the methods comprising administering a radiolabeled antibody conjugate of the present disclosure to the subject, and localizing the administered radiolabeled antibody conjugate in the tumor by PET imaging wherein presence of the radiolabeled antibody conjugate in the tumor identifies the subject as suitable for anti-tumor therapy comprising an inhibitor of the HGF/MET signaling pathway.

Anti-tumor therapy useful according to the methods disclosed herein can be any therapeutically useful inhibitor of the HGF/MET signaling pathway, i.e. an inhibitor of HGF, an inhibitor of MET, or an inhibitor of EKR or any other downstream protein in the HGF/MET signaling pathway. In some aspects, the anti-tumor therapy comprises an anti-MET antibody or antigen-binding fragment thereof, for example, any one or more of the antibodies listed in Table 1. In some aspects, the anti-tumor therapy comprises any one or more of the MET×Met bispecific antibodies, for example, any one of the bispecific antibodies listed in Table 2. In some aspects, antibody, bispecific antibody, or antigen-binding fragment thereof is conjugated to a drug (i.e. an ADC) useful for treating cancer. Exemplary anti-MET ADCs are disclosed in US-2018-0134794 A1.

According to one aspect, the present disclosure provides methods for identifying a candidate subject for anti-tumor therapy comprising an inhibitor of the HGF/MET signaling pathway, the methods comprising administering a radiolabeled antibody conjugate of the present disclosure to a subject having a tumor, and localizing the administered radiolabeled antibody conjugate in the tumor by PET imaging wherein presence of the radiolabeled antibody conjugate in the tumor identifies the subject as suitable for anti-tumor therapy comprising an inhibitor of the HGF/MET signaling pathway.

According to one aspect, the present disclosure provides methods for predicting response of a subject to an anti-tumor therapy, the methods comprising determining if the tumor is MET-positive, wherein if the tumor is MET positive it predicts a positive response of the subject to an anti-tumor therapy. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is MET-positive.

According to one aspect, the present disclosure provides methods for predicting response of a subject having a solid tumor to an anti-tumor therapy, the methods comprising determining if the tumor is MET positive, wherein a positive response of the subject is predicted if the tumor is MET positive. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is MET positive.

According to one aspect, the present disclosure provides methods for detecting a MET-positive tumor in a subject. The methods, according to this aspect, comprise administering a radiolabeled antibody conjugate of the present disclosure to the subject; and determining localization of the radiolabeled antibody conjugate by PET imaging, wherein presence of the radiolabeled antibody conjugate in a tumor indicates that the tumor is MET-positive. In some aspects, the method for detecting identifies the location of the tumor. In some aspects, the method for detecting permits monitoring progress of anti-tumor treatment, for example, whether the tumor regresses or stops growing.

According to one aspect, the present disclosure provides methods for determining the size of a MET-positive tumor in a subject. The methods, according to this aspect, comprise administering a radiolabeled antibody conjugate of the present disclosure to the subject; and visualizing the radiolabeled antibody conjugate by PET imaging, wherein the size of the tumor can be determined.

Provided herein are also methods for determining the presence of MET expressing cells in a subject. The methods comprise administering a radiolabeled anti-MET antibody conjugate or MET×MET bispecific antibody conjugate described herein to the subject and visualizing MET expression by PET imaging.

Provided herein are methods for diagnosing and treating a subject with a tumor, the methods comprising administering a radiolabeled conjugate described herein to the subject wherein localization of the radiolabeled antibody conjugate is imaged via PET imaging to determine if the tumor is MET-positive; diagnosing the subject with a MET-positive tumor; and administering to the subject an anti-tumor therapy comprising an inhibitor of the HGF/MET signaling pathway.

Provided herein are methods for diagnosing a subject having a MET expressing tumor, the methods comprising administering a radiolabeled anti-MET antibody conjugate or MET×MET bispecific antibody conjugate described herein to the subject; visualizing MET expression by PET imaging; and diagnosing the subject with a MET expressing tumor when MET expression is visualized by PET imaging.

As used herein, the expression “a subject in need thereof” means a human or non-human mammal that exhibits one or more symptoms or indications of cancer, and/or who has been diagnosed with cancer, including a solid tumor and who needs treatment for the same. In many embodiments, the term “subject” may be interchangeably used with the term “patient”. For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker (e.g., CA125). The expression includes subjects with primary or established tumors. In specific embodiments, the expression includes human subjects that have and/or need treatment for a solid tumor, e.g., colon cancer, breast cancer, lung cancer, prostate cancer, skin cancer, liver cancer, bone cancer, ovarian cancer, cervical cancer, pancreatic cancer, head and neck cancer, and brain cancer. The term includes subjects with primary or metastatic tumors (advanced malignancies). In certain embodiments, the expression “a subject in need thereof” includes patients with a solid tumor that is resistant to or refractory to or is inadequately controlled by prior therapy (e.g., treatment with an anti-cancer agent). For example, the expression includes subjects who have been treated with one or more lines of prior therapy such as treatment with chemotherapy (e.g., carboplatin or docetaxel). In certain embodiments, the expression “a subject in need thereof” includes patients with a solid tumor which has been treated with one or more lines of prior therapy but which has subsequently relapsed or metastasized.

In certain embodiments, the methods of the present disclosure are used in a subject with a solid tumor. The terms “tumor”, “cancer” and “malignancy” are interchangeably used herein. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and lymphomas. In certain embodiments, the term “solid tumor” includes cancers including, but not limited to, colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophageal cancer, head and neck cancer, salivary gland cancer, and myeloma.

In some aspects, the tumor is selected from the group consisting of acute myelogenous leukemia, adult T-cell leukemia, astrocytomas, cholangiocarcinoma, chronic myeloid leukemia, gastric cancer (e.g., gastric cancer with MET amplification), glioblastomata, head and neck cancer (e.g., head and neck squamous cell carcinoma [HNSCC]), Kaposi's sarcoma, leiomyosarcomas, lung cancer (e.g., non-small cell lung cancer [NSCLC]), lymphomas, malignant gliomas, malignant mesothelioma, melanoma, mesothelioma, MFH/fibrosarcoma, multiple myeloma, nasopharyngeal cancer, osteosarcoma, pancreatic carcinoma, prostate cancer, renal cell carcinoma, rhabdomyosarcoma, small cell lung cancer, synovial sarcoma, thyroid cancer, and Wilms' tumor.

According to one aspect, the present disclosure provides methods of treating a tumor in a subject. The methods, according to this aspect, comprise determining that the tumor is MET-positive; and administering one or more doses of an inhibitor of the HGF/MET signaling pathway. In some aspects, the inhibitor is an anti-MET antibody, a MET×MET bispecific antibody, or a drug conjugate thereof. In certain embodiments, the tumor is determined to be MET-positive by administering a radiolabeled antibody conjugate of the present disclosure to the subject; and visualizing the radiolabeled antibody conjugate in the tumor by PET imaging. Presence of the radiolabeled antibody conjugate in the tumor indicates that the tumor is MET-positive. In some embodiments, the cancer is lung cancer. In some embodiments, the cancer is non-small cell lung cancer. In some embodiments, the cancer is gastric cancer.

As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, and/or to increase duration of survival of the subject.

According to one aspect, the present disclosure provides methods for monitoring the efficacy of an anti-tumor therapy in a subject undergoing treatment for cancer, wherein the methods comprise administering a radiolabeled antibody conjugate of the present disclosure to the subject; imaging the localization of the administered radiolabeled conjugate in the tumor by PET imaging; and determining tumor growth, wherein a decrease from the baseline in radiolabeled signal indicates tumor regression and efficacy of the anti-tumor therapy. In certain embodiments, the anti-tumor therapy comprises an inhibitor of the HGF/MET signaling pathway (e.g., a MET×MET bispecific antibody).

As used herein, the term “baseline,” with respect to the MET expression in the tumor, means the numerical value of uptake of the radiolabeled conjugate for a subject prior to or at the time of administration of a dose of anti-tumor therapy. The uptake of the radiolabeled conjugate is determined using methods known in the art (see, for example, Oosting et al 2015, J. Nucl. Med. 56:63-69). In certain embodiments, the anti-tumor therapy comprises an inhibitor of the HGF/MET signaling pathway.

To determine whether there is tumor regression, the uptake of the radiolabeled conjugate is quantified at baseline and at one or more time points after administration of the inhibitor of the HGF/MET signaling pathway (e.g., a MET×MET bispecific antibody). For example, the uptake of the administered radiolabeled antibody conjugate (e.g., radiolabeled MET×MET bispecific antibody conjugate) may be measured at day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the inhibitor of the HGF/MET signaling pathway (e.g., a MET×MET bispecific antibody). The difference between the value of the uptake at a particular time point following initiation of treatment and the value of the uptake at baseline is used to establish whether there has been a difference in amount of tumor tissue (tumor regression or progression). For example, a decrease from baseline in the uptake upon treatment with at least one dose of the inhibitor of the HGF/MET signaling pathway means tumor regression and indicates efficacy of the anti-tumor therapy.

In certain embodiments, the radiolabeled antibody conjugate is administered intravenously or subcutaneously to the subject. In certain embodiments, the radiolabeled antibody conjugate is administered intra-tumorally. Upon administration, the radiolabeled antibody conjugate is localized in the tumor. The localized radiolabeled antibody conjugate is imaged by PET imaging and the uptake of the radiolabeled antibody conjugate by the tumor is measured by methods known in the art. In certain embodiments, the imaging is carried out 1, 2, 3, 4, 5, 6 or 7 days after administration of the radiolabeled conjugate. In certain embodiments, the imaging is carried out on the same day upon administration of the radiolabeled antibody conjugate.

In certain embodiments, the radiolabeled anti-MET conjugate can be administered at a dose of about 0.1 mg/kg of body weight to about 100 mg/kg of body weight of the subject, for example, about 0.1 mg/kg to about 50 mg/kg, or about 0.5 mg/kg to about 25 mg/kg, or about 0.1 mg/kg to about 1.0 mg/kg of body weight.

### IV.Examples

Certain embodiments of the disclosure are illustrated by the following non-limiting examples.

### Example 1: Generation of Human Antibodies to MET

Human anti-MET antibodies, including those listed in Table 1, were prepared and characterized as described in US-2018-0134794, which is incorporated herein by reference in its entirety. In brief, human antibodies to MET were generated using an immunogen comprising recombinant human MET extracellular domain fused to human Fc (R&D Systems, Catalog #358-MT, Minneapolis, MN). The mice used for the immunizations express a “universal light chain.” That is, the antibodies produced in this mouse have different heavy chain variable regions but essentially identical light chain variable domains.

The antibody immune response was monitored by a MET-specific immunoassay. When a desired immune response was achieved splenocytes were harvested and fused with mouse myeloma cells to preserve their viability and form hybridoma cell lines. The hybridoma cell lines were screened and selected to identify cell lines that produce MET-specific antibodies. Using this technique, and the immunogen described above, several anti-MET chimeric antibodies (i.e., antibodies possessing human variable domains and mouse constant domains) were obtained. In addition, several fully human anti-MET antibodies were isolated directly from antigen-positive B cells without fusion to myeloma cells, as described in U.S. 2007/0280945. Exemplary antibodies generated in this manner were designated as H4H13290P2, H4H13291P2, H4H13295P2, H4H13299P2, H4H13300P2, H4H13301P2, H4H13302P2, H4H13306P2, H4H13309P2, H4H13311P2, H4H13312P2, H4H13313P2, H4H13316P2, H4H13318P2, H4H13319P2, H4H13325P2, and H4H13331P2; sequences for these antibodies are shown in Table 1, above.

MET×MET bispecific antibodies were constructed from the anti-MET antibodies of Table 1. All anti-MET antibodies described herein comprise the same (“common”) light chain (comprising the light chain variable region [LCVR] amino acid sequence of SEQ ID NO: 138, and light chain CDR [LCDR1, LCDR2 and LCDR3] amino acid sequences of SEQ ID NOs: 140, 142 and 144). In addition, all of the bispecific antibodies illustrated in this Example contain a “D2” arm derived from the exemplary anti-MET antibody H4H13312P2. Thus, both antigen-binding domains (D1 and D2) of all of the bispecific antibodies described in this example comprise this common light chain variable region, and all D2 binding arms comprise the heavy chain variable region from H4H13312P2; however, the bispecific antibodies differ from one another in terms of their D1 heavy chain variable regions (HCVRs) and heavy chain CDRs (HCDRs). Exemplary antibodies generated in this manner were designated as H4H14634D, H4H14635D, H4H14636D, H4H14637D, H4H14638D, H4H14639D, H4H14640D, H4H14641D, H4H16445D, H4H16446D, H4H16447D, H4H16448D, and H4H16449D; sequences for these antibodies are shown in Table 2, above. For example, the MET×MET bispecific antibody H4H14639D comprises D1-HCVR (SEQ ID NO: 58) from the anti-MET antibody of H4H13306 and D2-HCVR (SEQ ID NO: 82) from the anti-MET antibody of H4H13312P2.

### Example 2: Conjugation of a MET×MET Bispecific Antibody H4H14639D with p-SCN-Bn-DFO

To modify the parental MET×MET bispecific antibody, H4H14639D, and an isotype control antibody to be suitable for ImmunoPET studies with radiolabeling, a chelator, p-SCN-bn-Deferoxamine (DFO, aka desferrioxamine; Macrocylics, Cat #: B-705), was attached to the antibodies.

For the modification, 100 mg of 26.1 mg/mL H4H14639D was split into four aliquots and buffer exchanged into conjugation buffer (150 mM NaCl, 50 mM sodium carbonate, pH 9.0; Sigma-Aldrich, Cat. #: S6297-1 KG and Gibco, Cat. #: 24740-011, respectively) via four pre-equilibrated PD-10 desalting columns (GE Healthcare, Cat. #: 17-0851-01), as per the manufacturer's instructions. The elution products were combined, and the concentration was determined by UV absorption spectroscopy (Thermo Scientific NanoDrop 2000c, Cat. #: ND-2000c-US-CAN) measured at 280 nm and calculated from the primary-sequence-based extinction coefficient. This elution product was further diluted to 10.4 mg/ml with the conjugation buffer. In a separate vial, p-SCN-Bn-DFO was prepared in neat anhydrous dimethyl sulfoxide (DMSO; Sigma-Aldrich, Cat #: 276855-100ML) at a concentration of 13.8 mM. The p-SCN-Bn-DFO solution was added to the diluted elution product in 1/4 increments, mixed by gentle pipetting, such that the final reaction solution makeup was 10 mg/mL bispecific antibody in conjugation buffer, 2% DMSO and 4-fold mole-to-mole excess of p-SCN-Bn-DFO to bispecific antibody. This solution was allowed to incubate in a 37° C. water bath with no additional agitation. After 30 minutes at 37° C., the reaction solution was split into four aliquots and promptly passed through four PD-10 desalting columns pre-equilibrated with a buffer containing 50 mM sodium acetate at pH 5.0 (formulation buffer; Sigma-Aldrich, Cat #32319-1 KG-R). The final elution solutions were combined and sterile-filtered via a syringe filter (Acrodisc 13 mm syringe filter, Pall Corporation, Cat #: 4602) and referred to as the DFO-Ab immunoconjugate, DFO-H4H14639D immunoconjugate.

The concentration and DFO-to-Antibody Ratio (DAR, i.e. chelator-to-antibody ratio) was subsequently measured by UV absorption spectroscopy. For the absorbance measurement, the DFO-conjugated antibody was measured against the formulation buffer at 252 nm (A252), 280 nm (A280) and 600 nm (A600); see Tables 3 and 4. For the calculation, the background was corrected at each absorbance value using the equation:

$${A}_{λ}^{′} = {A}_{λ} - {A}_{600}$$

The antibody concentration, conjugate concentration, and DAR were calculated using the equations below: MW=144950 g mol⁻¹, ε280=207729 M⁻¹ cm⁻¹, ε252=79048 M⁻¹ cm⁻¹.

### Antibody Concentration Calculation

$$Conc mAb ( mg / mL ) = \frac{{A}_{280}^{′}}{{ϵ}_{280}} * MW$$

### Conjugate Concentration Calculation

$$Conc conjugate ( mg / mL ) = \frac{{A}_{252}^{′} - 1.53 {A}_{280}^{′}}{{ϵ}_{252} - 1.53 {ϵ}_{280}} * MW$$

### DAR Calculation

$$DAR = \frac{{ϵ}_{252} {A}_{280}^{′} - {ϵ}_{280} {A}_{252}^{′}}{18800 {A}_{252}^{′} - 28700 {A}_{280}^{′}}$$

The final DFO-Ab immunoconjugate yield was 61 mg.

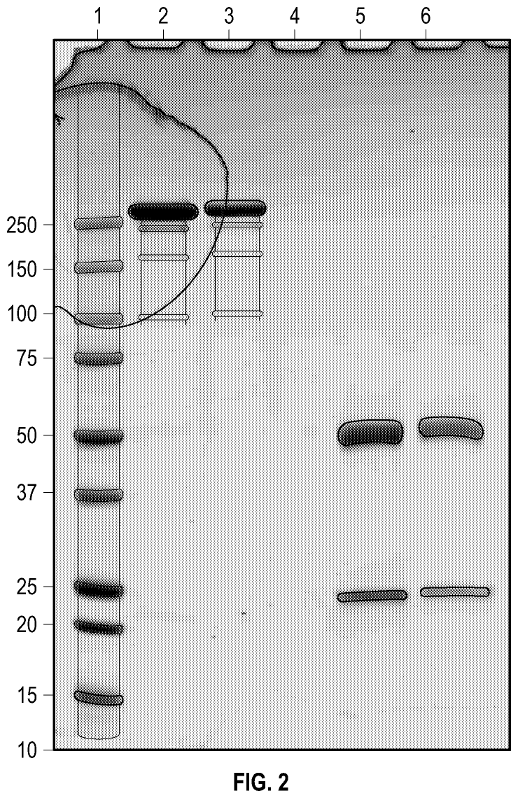

The DFO-Ab immunoconjugate was assayed for monomeric purity with size-exclusion high performance liquid chromatography (SE-HPLC), using a Superdex 200 Increase 10/300 GL column (GE Healthcare, Cat. #: 28990944) with inline UV absorbance detector monitored at 280 nm and a PBS mobile phase at 0.75 mL/min (see FIG. 1). The main elution peak at approximately 15 minutes corresponds the monomeric species. The DFO-Ab immunoconjugate was also evaluated by sodium dodecyl sulfate polyacrylamide gel electrophoresis (SDS-PAGE; Invitrogen, Novex 4-20% Tris-Glycine Mini Gels, Cat. #: XP04200) against the unmodified bispecific antibody, H4H14639D, and executed as per manufacturer's instructions (see FIG. 2). Target binding equilibrium constant, i.e. Kp, of DFO-Ab immunoconjugate was assayed by SPR (GE Healthcare, Biacore 8k) and was determined to be within 10% of the bispecific antibody K_{D}.

TABLE 3
DAR, Concentration, and Monomeric Purity of Conjugate

| | | Concentration | |
|---|---|---|---|
| Antibody | DAR | (mg/mL) | % Monomeric |
| H4H14639D | 1.26 | 6.76 | 99.6% |

TABLE 4
Background-Subtracted UV Absorption Spectroscopy

| Antibody | A′₂₈₀ | A′₂₅₂ |
|---|---|---|
| H4H14639D | 1.08 | 0.538 |

The bispecific antibody was successfully conjugated with p-SCN-Bn-DFO via primary amine chemistry as shown by UV absorption spectroscopy, SE-HPLC and SDS-PAGE. The calculated DAR of 1.26 was within the expected range of 1.0 to 2.0. SE-HPLC chromatogram demonstrated a highly monomeric product (99.6%) with no detectable lower molecular weight species. SDS-PAGE demonstrated bispecific antibody remains unchanged after DFO-conjugation.

### Example 3: 89Zr chelation of DFO Conjugated Bispecific Antibodies

For use in ImmunoPET in vivo studies, the DFO-conjugated MET×MET bispecific antibody, H4H14639D, and a DFO-conjugated isotype control antibody were radiolabeled with ⁸⁹Zr.

DFO-conjugated antibody (250 μg) was first brought to a total volume of 200 μL with 1 M HEPES, pH 7.4 (Teknova, Cat. #: H1030). Separately, approximately 5 mCi (<150 uL)⁸⁹Zr-oxalic acid solution (3D Imaging, Little Rock AR) was neutralized and buffered to pH 6.8 to 7.4 by bringing up to a total volume of 1000 μl of 1 M HEPES, pH 7.4. The DFO-Ab immunoconjugate and buffered ⁸⁹Zr solutions were combined, then gently mixed by pipet and incubated at room temperature for 45 minutes, quiescent. Upon completion, the reaction mixture was promptly buffer exchanged by a PD-10 column (GE Healthcare, Cat. #: 17-0851-01), preconditioned with 250 mM sodium acetate, pH 5.5 (Sigma-Aldrich, Cat. #: 32319-1 KG-R), as per manufacturer's instructions. The concentration of the elution product, now referred to as DFO-Ab radioimmunoconjugate, was determined by UV absorption spectroscopy (Thermo Scientific NanoDrop 2000c, Cat. #: ND-2000c-US-CAN) measured at 280 nm and calculated from the on DFO-contribution-adjusted, primary-sequence-based extinction coefficient using the equation:

$$Concentration in mg / mL = Absorption at 280 nm in AU \div 1.86 mL / mg 1 / cm$$

The DFO-Ab radioimmunoconjugate was sterile filtered and assayed for protein yield, specific activity (SA), radiochemical purity (RCP), protein purity and the target specific binding, i.e. immunoreactivity (IR). Data is reported in Table 5. The activity yield in mCi was measured using the dose calibrator (Capintec CRC-25R; Cat #: 5130-3215). The protein yield and specific activity of DFO-Ab radioimmunoconjugate was determined using the following equations:

$$a . protein yield in mg = concentration in mg / mL \times mass of solution in grams$$

$$b . SA in mCi / mg = activity yield in mCi \div mass of conjugate in mg$$

The RCP, unincorporated ⁸⁹Zr, and protein purity were assayed by size-exclusion high performance liquid chromatography (SE-HPLC) using a Superdex 200 Increase 10/300 GL column (GE Healthcare, Cat. No. 28990944) with inline UV 280 absorption and gamma emission detector connected in series (Agilent Technologies, Model 1260 configured with Lablogic SCAN-RAM radiodetector) using PBS mobile phase at a flow rate of 0.75 mL/min. The percent protein purity was determined by comparing the relative integration of the high molecular weight (HMW) species peak (~10 to ~ 15 minutes) to the main peak (~15 to ~18 minutes) in the UV 280 chromatogram. Low molecular weight species (~18 to ~25 minutes) were not observed. The radio-chromatogram (gamma emission) was used to determine the radiochemical purity by relative comparison of the integration of the main to the unincorporated ⁸⁹Zr peak (~ 25 min) and % HMW species.

IR of the DFO-Ab radioimmunoconjugate was measured by a cell binding assay requiring two 500 μL aliquots, A and B, of EBC-1 cells (JCRB No. JCRB0820) at A at 2.0×10⁷ cells/mL and B at 0.5 ×10⁷ cells/mL. The DFO-Ab radioimmunoconjugate (20 ng) was added to aliquot A and allowed to incubate at 37° C., 5% CO₂ for 45 minutes. Both aliquots A and B were centrifuged (Eppendorf; Model #5504R) at 1500 rpm for 5 minutes. The supernatant from cell pellet B was discarded. The supernatant from cell pellet A was transferred to cell pellet B, followed by incubation and separation as above. Each cell pellet (A and B) was washed twice with 1 mL fresh cell culture media, centrifuging at 1500 rpm for 5 minutes between each wash. Supernatants from the washes were collected. The final activities for all components (each cell pellet resuspended in 500 μL of cell culture media, the supernatant and the four wash supernatants) were measured with a gamma counter (Perkin Elmer Wizard2; Model #2470-0020). IR was determined by sum of both pellets' activity divided by sum of the activity of all components, times 100%. This process was tested against a non-specific DFO-Ab radioimmunoconjugate (n=1) and the IR was determined to be 2.8%.

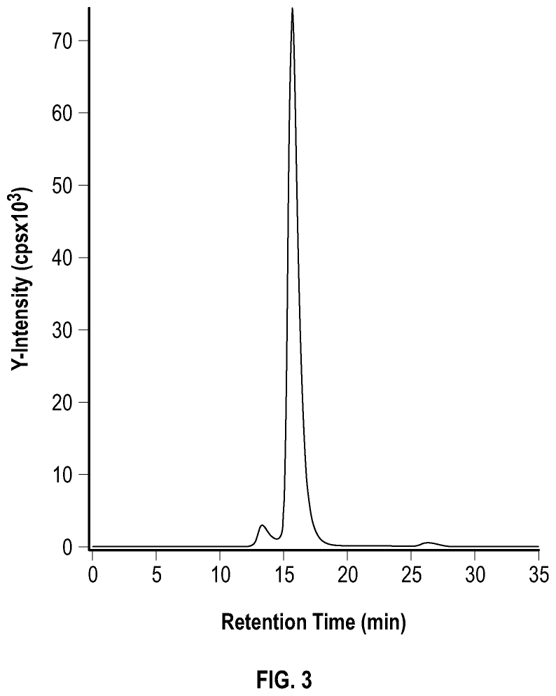

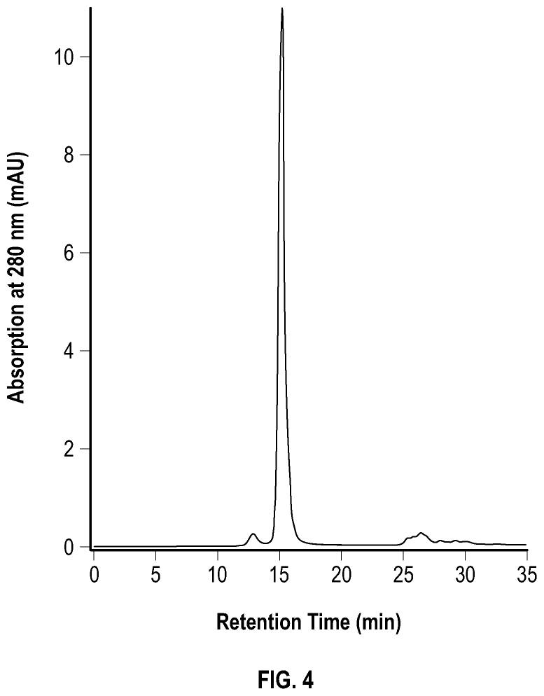

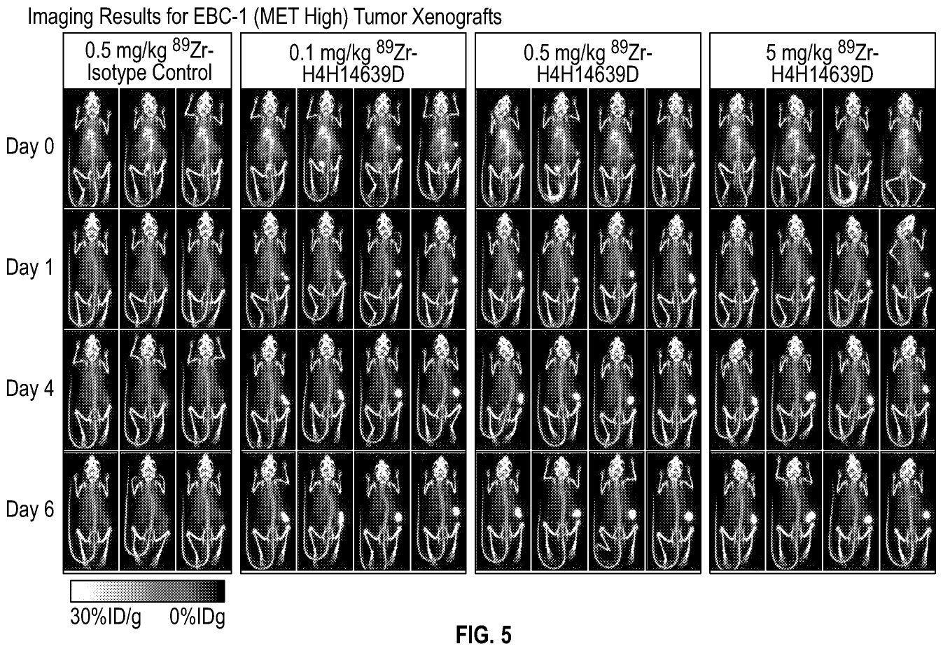

Assay results for the DFO-Ab radioimmunoconjugates generated above (n=5) are recorded in Table 5. Notably the average RCP was 94.6±1.2% with 2.7±1.9% unincorporated ⁸⁹Zr present and protein purity was 98.3±1.5%. Representative chromatograms are shown in FIGS. 3 and 4, respectively. The SA ranged from 11.9 to 21.3 mCi/mg and was appropriate for in vivo dosing. The average IR was 84±7% whereas a non-specific control was 2.8%.

TABLE 5
Summary of DFO-Ab radioimmunoconjugate for
in vivo imaging and biodistribution studies

| Radio- | | | | | | |
|---|---|---|---|---|---|---|
| labeling/ | | | | | | Average ± |
| Study # | 1 | 2 | 3 | 4 | 5 | Stdev |
| Concen- | 0.100 | 0.103 | 0.120 | 0.115 | 0.120 | 0.11 ± |
| tration | | | | | | 0.01 |
| (mg/mL) | | | | | | |
| Protein yield | 171 | 168 | 203 | 196 | 209 | 189 ± |
| (ug) | | | | | | 19 |
| SA | 16.0 | 11.9 | 21.3 | 12.8 | 17.2 | 15.8 ± |
| (mCi/mg) | | | | | | 3.4 |
| RCP | 94.6 | 92.3 | 94.8 | 95.7 | 95.4 | 94.6 ± |
| (%) | | | | | | 1.2 |
| Unincor- | 5.4 | 4.6 | 1.1 | 1.4 | 0.8 | 2.7 ± |
| porated | | | | | | 1.9 |
| ⁸⁹Zr | | | | | | |
| Protein | ND | 98.2 | 95.9 | 99.5 | 99.5 | 98.3 ± |
| Purity | | | | | | 1.5 |
| (%) | | | | | | |
| Appearance | Clear | Clear | Clear | Clear | Clear | NA |
| IR (%) | 91 | ND | 85 | 78 | 86 | 84 ± |
| | | | | | | 7 |

Stdev: standard deviation. NA: not applicable. ND: not determined. mAb: H4H14639D

### Example 4: Binding Affinities and Kinetic Constants of MET×MET Bispecific and DFO-conjugated MET×MET Bispecific

Equilibrium dissociation constants (K_{D} values) for hMET.mmh binding to purified anti-MET×MET bispecific mAb (H4H14639D) or anti-MET×MET bispecific mAb conjugated to DFO (H4H14639D-DFO) were determined using a real-time surface plasmon resonance biosensor using a Biacore T-200 instrument. The CM5 Biacore sensor surface was derivatized by amine coupling with a monoclonal mouse anti-human Fc antibody to capture purified H4H14639D or H4H14639D-DFO. This Biacore binding study was performed in a buffer composed of 0.01M HEPES pH 7.4, 0.15M NaCl, 0.05% v/v Surfactant P20 (HBS-P running buffer). Different concentrations of hMET with a C-terminal myc.myc hexahistidine tag prepared in HBS-ET running buffer were injected over the antibody captured surface at a flow rate of 50 μL/minute. Association of hMET.mmh to the captured monoclonal antibody was monitored for 5 minutes and the dissociation of hMET.mmh in HBS-ET running buffer was monitored for 10 minutes. All of the binding kinetics experiments were performed at 25° C. Kinetic association (ka) and dissociation (ka) rate constants were determined by fitting the real-time sensorgrams to a 1:1 binding model using Scrubber 2.0c curve fitting software. Binding dissociation equilibrium constants (K_{D}) and dissociative half-lives (t1/2) were calculated from the kinetic rate constants as:

$${K}_{D} ( M ) = {k}_{d} / {k}_{a} , and t \frac{1}{2} ( min ) = 0 \text{.693} / {k}_{d} / 60$$

Binding kinetic parameters for human MET binding to purified antibodies at 25° C. are shown below in Table 6.

TABLE 6
Human MET Binding Kinetics to MET × MET Bispecific Antibody and DFO
Conjugated MET × MET Bispecific Antibody at 25 °C

| | mab | Antigen | Antigen | | | | | | Theoretical | % Bound/ |
|---|---|---|---|---|---|---|---|---|---|---|
| Common | Captured | Bound | Conc | ka | kd | KD | t½ | Chi\hat{ } | Rmax | Theoretical |
| Name | (RU/nm) | (RU/nm) | (nM) | (1/Ms) | (1/s) | (M) | (min) | 2 | (RU) | Rmax |
| Non-binding | 70.5 ± | 0.25 | 90 | NB | NB | NB | NB | NB | NB | NB |
| isotype | 13.1 | | | | | | | | | |
| Control | | | | | | | | | | |
| H4H14639D | 49.3 ± | 26.35 | 90 | 3.25E+05 | 2.72E−03 | 8.36E−09 | 4.25 | 0.469 | 71.4 | 36.92 |
| (unconjugated) | 1.0 | | | | | | | | | |
| H4H14639D- | 49.1 ± | 26.68 | 90 | 3.02E+05 | 2.39E−03 | 7.90E−09 | 4.84 | 0.422 | 71.1 | 37.54 |
| DFO | 10.8 | | | | | | | | | |
| DAR 0.59 | | | | | | | | | | |
| Non-binding | 120.1 ± | −1.46 | 90 | NB | NB | NB | NB | NB | NB | NB |
| isotype | 3.3 | | | | | | | | | |
| Control | | | | | | | | | | |
| H4H14639D- | 82.3 ± | 46.99 | 90 | 2.84E+05 | 1.83E−03 | 6.47E−09 | 6.30 | 0.656 | 119.1 | 39.44 |
| DFO | 2.4 | | | | | | | | | |
| DAR 1.02 | | | | | | | | | | |
| H4H14639D- | 83.3 ± | 48.39 | 90 | 3.69E+05 | 1.62E−03 | 4.38E−09 | 7.14 | 0.661 | 120.6 | 40.13 |
| DFO | 1.8 | | | | | | | | | |
| DAR 2.14 | | | | | | | | | | |

### Example 5: In vivo Biodistribution of Radiolabeled MET×MET Bispecific Antibody

Tumor xenografts that differ based on MET expression levels, EBC1 (MET high), NCI-H441 (MET moderate) and NCI-H358 (MET low), were selected for imaging in immunocompromised mice. SCID mice were implanted with 5×106 tumor cells and allowed to grow for 10-14 days. Mice were then dosed with 0.1 mg/kg ⁸⁹Zr-DFO-H4H14639D and increasing amounts of unlabeled unconjugated antibody to achieve final protein doses of 0.1, 0.5 and 5 mg/kg. Control animals were dosed with 0.1 mg/kg ⁸⁹Zr-DFO-Isotype Control Antibody and unlabeled unconjugated antibody to a final protein dose of 0.5 mg/kg. PET Imaging was conducted at Days 0, 1, 4 and 6. Biodistribution was performed at Day 6.

A Sofie Biosciences G8 PET/CT was used to acquire PET/CT images (Sofie Biosciences and Perkin Elmer). The instrument was pre-calibrated for detection of ⁸⁹Zr prior to image acquisition. The energy window ranged from 150 to 650 keV with a reconstructed resolution of 1.4 mm at the center of the field of view. Mice underwent induction anesthesia using isoflurane and were kept under continuous flow of isoflurane during imaging. Static 10-minute images were acquired using the G8 acquisition software and subsequently reconstructed using the pre-configured settings. Image data was corrected for decay and other parameters. CT images were acquired following PET acquisition and subsequently co-registered with the PET images. Images were prepared using VivoQuant post-processing software (inviCRO Imaging Services).

For biodistribution studies, mice were euthanized at the final time-point (6 days post-⁸⁹Zr-DFO-H4H14639D administration) and blood was collected via cardiac puncture. Tumors and normal tissues were then excised, placed in counting tubes, and weighed. Count data for ⁸⁹Zr in CPM was then collected by measuring samples on an automatic gamma counter (Wizard 2470, Perkin Elmer). The percent-injected dose per gram (% ID/g) was calculated for each sample using standards prepared from the injected material.

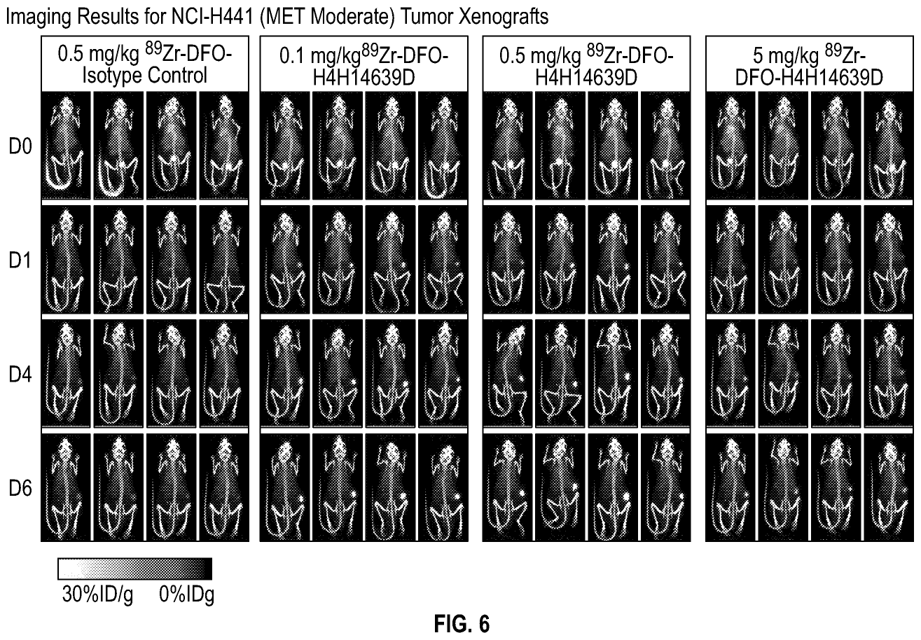

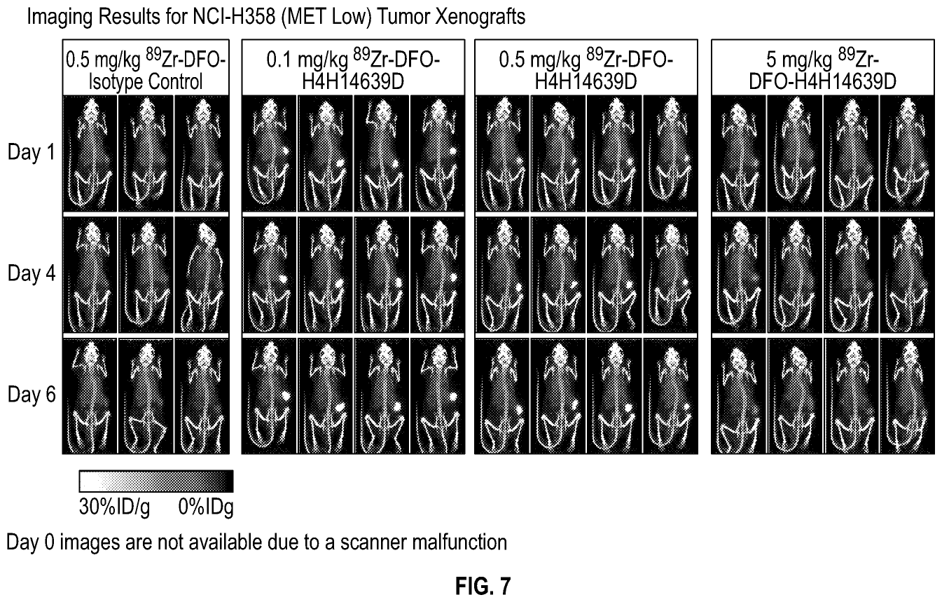

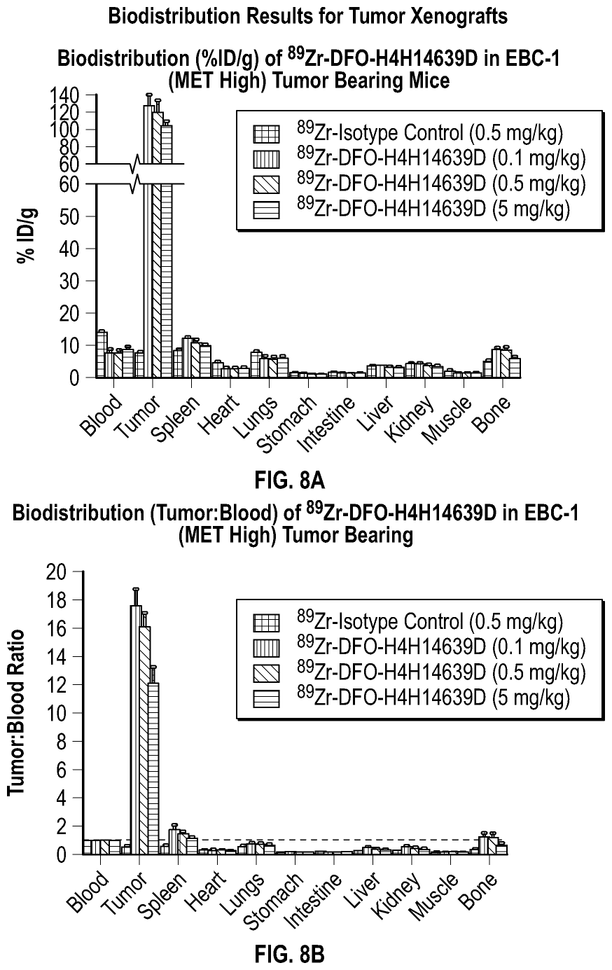

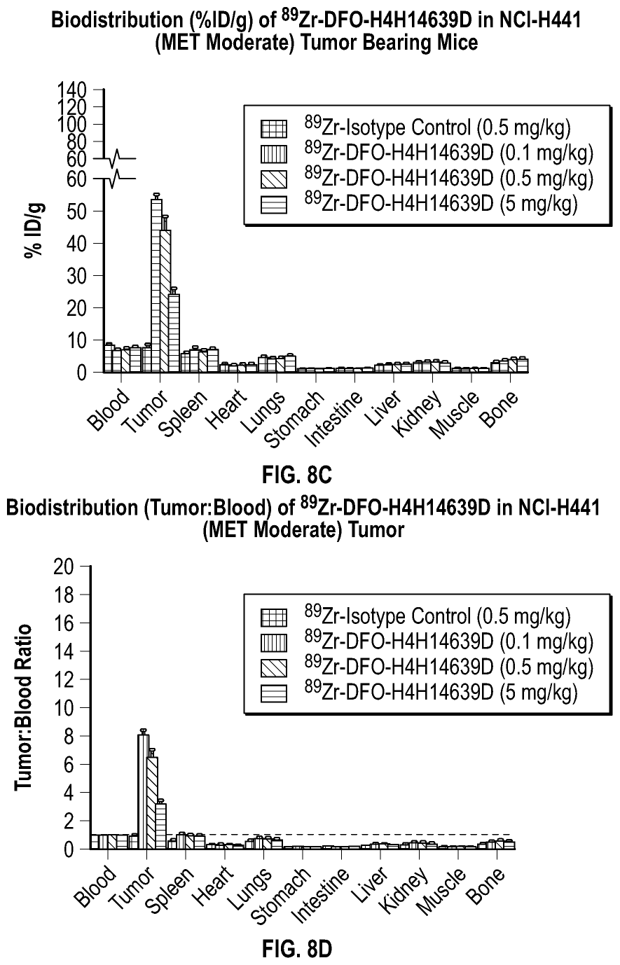

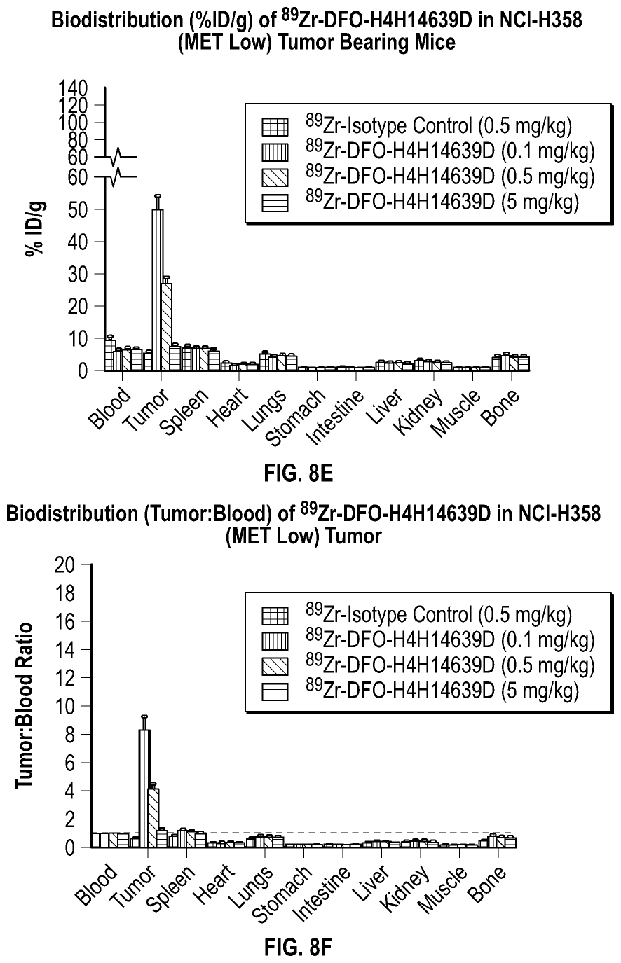

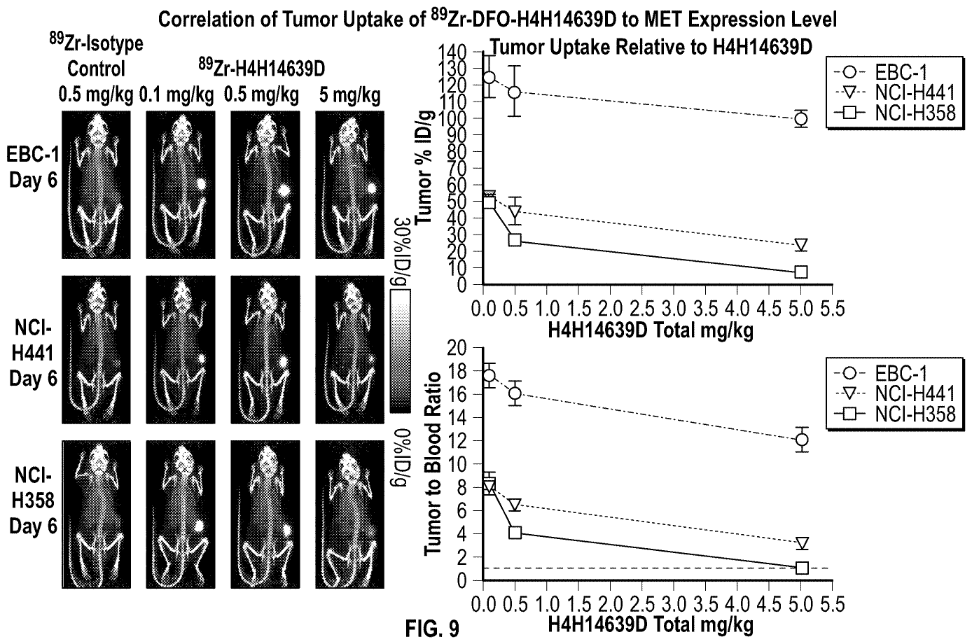

The imaging results demonstrate that ⁸⁹Zr-DFO-H4H14639D specifically localizes to MET expressing tumor xenografts (FIGS. 5-7), which is further demonstrated by the biodistribution data. The blocking dose of 5 mg/kg ⁸⁹Zr-DFO-H4H14639D showed increased blood uptake (% ID/g) and lower tumor uptake (% ID/g) in NCI-H441 (MET Moderate) and NCI-H358 (MET Low) tumors compared to the lower doses of 0.1 and 0.5 mg/kg ⁸⁹Zr-DFO-H4H14639D (FIG. 8). Overall tumor uptake of ⁸⁹Zr-DFO-H4H14639D in different tumor xenografts demonstrates a good correlation to relative MET expression (FIG. 9).

### Example 6: MET×MET Bispecific Antibody Binding Capacity (ABC) by the Saturation Radioligand Binding Assay

An Antibody Binding Capacity (ABC) assay was performed on the EBC-1, NCI-H441, NCI-H358 cell lines using ⁸⁹Zr-DFO-anti-Met single-arm comparator antibody (Comp1; Onartuzumab, mentioned in U.S. 2016/0222115 and Martens et al., Clin Cancer Res 2006, 12 (20): 6144-6152) and ⁸⁹Zr-DFO-H4H14639D as the radioligands. Examples of radioligand preparation (the DFO antibody conjugation and subsequent Zr⁸⁹-radiolabeling) are described vide supra. All six experiments were performed in similar fashion with the aid of a Hamilton Starlet liquid handling system. In short, cells were first harvested, assayed for viability (>90% confirmed by trypan blue exclusion staining), and brought to 2 to 5 ×10⁶ cells/mL in complete media. Next, 100 μL (0.2 to 0.5 million cells, N_{C}) was aliquoted along duplicate or quadruplicate rows of a V-bottom 96-well plate (“cell plate”). The cell plate was held at 4° C. until dosed with radiolabeled antibody in a later step. In a second V-bottom 96-well plate (“mAb plate”), 150 μL of radiolabeled antibody (0.10 to 0.12 mg/mL) was aliquoted across the rows of column 1 as well as a single well in column 12 as an internalization control. Columns 2-9 were then serial diluted by a factor 2.8 with cold media. Next, 50 μL of mAb plate was stamped into the cell plate. The cell plate was incubated with gentle agitation for 45 minutes at 4° C. After incubation, wells from columns 1 through 9 were first gently mixed by pipet to make a momentary cell suspension, then 30 μL (or 20% of total) was collected from each well into flip-cap tubes representing the total antibody dosed. The remaining cell plate was spun at 150 g for 5 minutes before removing the supernatants and discarding. The cell plate was then washed with 200 μL cold media, with five aspirate/dispense cycles before spinning the plate again, then removing the supernatants and discarding. This wash process was repeated two more times, before resuspending cells in 200 μL of cold buffer (10% FBS in PBS, v/v). Of the 200 μL, 180 μL from each well was collected and dispensed into flip-cap tubes, representing the cell-bound antibody. The activity of the total and bound antibody samples was measured using a gamma counter (Hidex Automatic Gamma Counter, Model #425-601). A calibration curve was generated from the total antibody dosed samples using counts detected vs total antibody dosed per well, T, based on the dilution series starting from the initial antibody concentration (divided by five because of sampling). The calibration curve was used to convert the bound fraction counts (multiplied by 1.38 because of sampling) to mass (or concentration) of bound antibody, B.

### Internalization Control:

The radioligand internalization was also assayed at the highest dosed concentration at 4° C. for the 45 minutes. If significant, the fraction of internalized antibody was used to scale ABC accordingly. For the internalization control, the entire well contents (200 μL) of terminal column 12 was transferred to a 1.5 mL Eppendorf tube containing 1 mL low pH stripping buffer (50 mM glycine, 150 mM NaCl, pH 2.4) and left at room temperature for ten minutes. After the ten-minute incubation, the Eppendorf tube was spun at 150 g for 5 minutes. The supernatant was removed without disturbing the cell pellet and reserved for counting. The cell pellet was washed with 1 mL cold buffer (10% FBS in PBS, v/v) by aspirating 10 times, spinning, removing, and reserving the supernatant between washes. Activity of cell pellets, stripping supernatants, and wash supernatants were measured using a gamma counter. The fraction internalized, I, was calculated as the ratio of activity of the cell pellet over sum of the total activity of the cell pellet, stripping supernatant and wash supernatants.

### ABC of Comp1:

Binding saturation data was fit using Equation 1 assuming the law of mass action under single-site conditions. The bound value was determined and converted to ABC via Equation 2. Under our binding/washing protocol, non-specific binding was determined to be a negligible component for all runs and was not considered as a part of the analysis. Internalization was determined to have a negligible contribution to the bound radioactivity and was also not considered as part of the analysis.

$$B = \frac{1}{2} ( {K}_{D} + {T}_{Ag} + T - \sqrt{{( {K}_{D} + {T}_{Ag} + T )}^{2} - 4 {T}_{ag} T} ) , \qquad Equation 1 ( for Comp1 )$$

where, K_{D} is the equilibrium disassociation constant in nM (fit parameter), T_{Ag} is the total Ag present in nM (fit parameter), T is the total antibody dosed in nM (measured), B is the bound concentration in nM (measured).

$$ABC = \frac{{N}_{A} {T}_{Ag} V}{{N}_{c} {10}^{15}} , \qquad Equation 2$$

where N_{A} is Avogadro's number, V is the well volume (150 μL), N_{C} is the number cells per well (known).

### ABC of H4H14639D:

Because there are multiple binding formats for H4H14639D, the data set was fit by the Hill-Langmuir equation (equation 3). The ABC was then calculated using equation 2. Non-specific binding was determined to be a negligible component for all runs was not considered as part of the analysis. The radioligand internalization was determined to have a non-negligible contribution to the bound radioactivity determination and therefore was compensated accordingly.

$$B = \frac{{T}_{Ag} ( 1 - I )}{1 + {( \frac{{K}_{l}}{T} )}^{n}} \qquad Equation 3 ( for H4H14639D )$$

where, B and T_{Ag} as above, I is the fraction internalized (measured), K_{I}, n, and T_{Ag} are fit parameters.

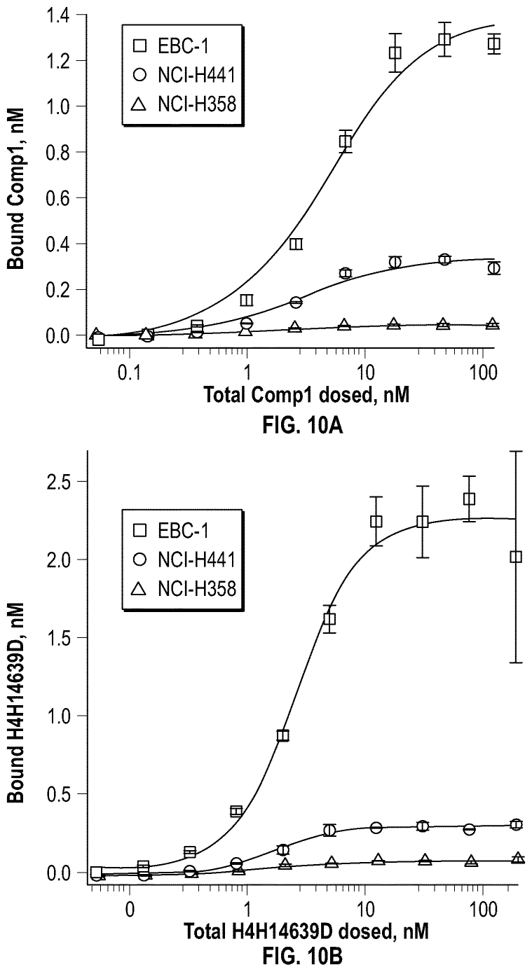

The ABC results are summarized in Tables 7 and 8 and shown in FIGS. 10A and 10B.

TABLE 7
ABC for Comp1 for three cell lines tested using equations
1 and 2. Fraction internalized at 4° C. was negligible.

| ABC/Cell line | EBC-1 | NCI-H441 | NCI-H358 |
|---|---|---|---|
| Average ± | 250 ± 10, 4 | 62 ± 3, 4 | 21 ± 4, 2 |
| standard | | | |
| deviation × | | | |
| 10³, n | | | |

TABLE 8
ABC for H4H14639D for three cell lines
tested using equation 2 and 3.

| ABC/Cell line | EBC-1 | NCI-H441 | NCI-H358 |
|---|---|---|---|
| Average ± | 310 ± 30, 4 | 46.25 ± 0.01, 2 | 37 ± 4, 2 |
| standard | | | |
| deviation × | | | |
| 10³, n | | | |
| Fraction | 0.237 | 0.172 | 0.09 |
| internalized under | | | |
| experimental | | | |
| conditions (4° C.) | | | |

The ABC value using Comp1, an anti-Met, single-armed/mono-valent format antibody, is a reasonable estimate of the Met receptor copy number (i.e. 1-to-1 antibody to receptor). However, the ABC value using the antibody H4H14639D is not expected to represent the 1-to-1 Met receptor copy number a priori.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

Informal Sequence Listing

| SEQ ID. NO. | SEQUENCE LISTING | Description |
|---|---|---|
| 1. | caggtgcagc tgcaggagtc gggcccagga ctggtgaagc cttcggagac cctgtccctc | DNA |
| | acctgcactg tctctggtga ctccatcagt agttactatt ggacctggat ccggcagccc | sequence |
| | ccagggaagg gactggagtg gattgggtat atcttttaca gggggggcac cacctacaac | |
| | ccctccctca agagtcgagt caccatatca gtagacacgt ccaagaacca gttctccctg | |
| | aagttgaggt ctgtgaccgc cgcagacacg gccgtgtatt actgtgcgag gggagacgat | |
| | cttttagtgg tgacaagtgt ctactggtac atcgatctct ggggccgtgg caccctggtc | |
| | accgtctcct ca | |
| 2. | QVQLQESGPG LVKPSETLSL TCTVSGDSIS SYYWTWIRQP PGKGLEWIGY IFYRGGTTYN | Amino Acid |
| | PSLKSRVTIS VDTSKNQFSL KLRSVTAADT AVYYCARGDD LLVVISVYWY IDLWGRGTLV | sequence |
| | TVSS | |
| 3. | ggtgactcca tcagtagtta ctat | DNA |
| | | sequence |
| 4. | GDSISSYY | Amino Acid |
| | | sequence |
| 5. | atcttttaca gggggggcac c | DNA |
| | | sequence |
| 6. | IFYRGGT | Amino Acid |
| | | sequence |
| 7. | gcgaggggag acgatctttt agtggtgaca agtgtctact ggtacatcga tctc | DNA |
| | | sequence |
| 8. | ARGDDLLVVT SVYWYIDL | Amino Acid |
| | | sequence |
| 9. | caggtgcagc tggtggagtc tgggggaggc gtggtccagc ctggggggtc cctgagactc | DNA |
| | tcctgtgcag cgtccggatt caccttcagt ggctatggca tgcactgggt ccgccaggct | sequence |
| | ccaggcaagg ggctggagtg gatggcagtt atatggtatg atggaagtaa tgattactat | |
| | ccagactccg tgaagggccg attcaccatc tccagagaca attccaagaa cacgctgtat | |
| | ctgcaaatga acagcctgag agtcgaggac acggctgtgt attactgtgc gcgagatgcg | |
| | tgggacctac tacgttcctt tgactactgg ggccagggaa ccctggtcac cgtctcctca | |
| 10. | QVQLVESGGG VVQPGGSLRL SCAASGFTFS GYGMHWVRQA PGKGLEWMAV IWYDGSNDYY | Amino Acid |
| | PDSVKGRFTI SRDNSKNTLY LQMNSLRVED TAVYYCARDA WDLLRSFDYW GQGTLVTVSS | sequence |
| 11. | ggattcacct tcagtggcta tggc | DNA |
| | | sequence |
| 12. | GFTFSGYG | Amino Acid |
| | | sequence |
| 13. | atatggtatg atggaagtaa tgat | DNA |
| | | sequence |
| 14. | IWYDGSND | Amino Acid |
| | | sequence |
| 15. | gcgcgagatg cgtgggacct actacgttcc tttgactac | DNA |
| | | sequence |
| 16. | ARDAWDLLRS FDY | Amino Acid |
| | | sequence |
| 17. | caggtgcagc tggtggagtc tgggggaggc gtggtccagc ctgggaggtc cctgagagtc | DNA |
| | tcttgtgtag tgtctggatt caccttcagc agctttggca tgcattgggt ccgccaggct | sequence |
| | ccagacaagg ggctggagtg ggtggcagtt atatggtatg atggaagtaa tgattactat | |
| | tcagactccg tgaagggccg attcaccatc tccagagaca attccaagaa cacgctgttt | |
| | ctacaaatga accgcctgag agccgaagac acggctgttt attactgtgc gcgagctaat | |
| | aactggaacc gttttgatgc ctttgatctc tggggccaag ggacaatggt caccgtctct | |
| | tca | |
| 18. | QVQLVESGGG VVQPGRSLRV SCVVSGFTFS SFGMHWVRQA PDKGLEWVAV IWYDGSNDYY | Amino Acid |
| | SDSVKGRFTI SRDNSKNTLF LQMNRLRAED TAVYYCARAN NWNRFDAFDL WGQGTMVTVS | sequence |
| | S | |
| 19. | ggattcacct tcagcagctt tggc | DNA |
| | | sequence |
| 20. | GFTFSSFG | Amino Acid |
| | | sequence |
| 21. | atatggtatg atggaagtaa tgat | DNA |
| | | sequence |
| 22. | IWYDGSND | Amino Acid |
| | | sequence |
| 23. | gcgcgagcta ataactggaa ccgttttgat gcctttgatc tc | DNA |
| | | sequence |
| 24. | ARANNWNRED AFDL | Amino Acid |
| | | sequence |
| 25. | gaggtgcagc tggtggagtc tgggggaggc ttggttcagc ctggagggtc cctgagactc | DNA |
| | tcctgtgcag cctctggatt catcttcagt aattatgaaa tgaactgggt ccgccaggct | sequence |
| | ccagggaagg gactggaatg gatttcatac attactagta gtggtaatat gaaatattac | |
| | gcagactctg tgaagggccg attcaccatc tccagagaca acgacaagaa ttcactgtat | |
| | ctgcaaatga gtagtctgag agtcgaggac acggctgttt attattgtgt gagaggaggg | |
| | cgatttttgg agtggttgac ctactacgtt atggtcgtct ggggccaagg gaccacggtc | |
| | accgtctcct ca | |
| 26. | EVQLVESGGG LVQPGGSLRL SCAASGFIFS NYEMNWVRQA PGKGLEWISY ITSSGNMKYY | Amino Acid |
| | ADSVKGRFTI SRDNDKNSLY LQMSSLRVED TAVYYCVRGG RFLEWLTYYV MVVWGQGTTV | sequence |
| | TVSS | |
| 27. | ggattcatct tcagtaatta tgaa | DNA |
| | | sequence |
| 28. | GFIFSNYE | Amino Acid |
| | | sequence |
| 29. | attactagta gtggtaatat gaaa | DNA |
| | | sequence |
| 30. | ITSSGNMK | Amino Acid |
| | | sequence |
| 31. | gtgagaggag ggcgattttt ggagtggttg acctactacg ttatggtcgt c | DNA |
| | | sequence |
| 32. | VRGGRFLEWL TYYVMVV | Amino Acid |
| | | sequence |
| 33. | caggtgcagc tggtggagtc tgggggaggc gtggtccagc ctgggaggtc cctgagactc | DNA |
| | tcctgtgcag tgtctggatt caccttcagt agctatggca tgcactgggt ccgccaggct | sequence |
| | ccaggcaagg ggctggagtg ggtggcaaat atttggtatg atggaactaa tgattactat | |
| | ccatactccg tgaagggccg attcaccatc tccagagaca attcccagaa cacactatat | |
| | ctgcaaatga acagcctgag agccgaggac acggctgtat attactgtgc gagagaggac | |
| | ttcattaact accggtcttt tgactattgg ggccagggaa ccctggtcac cgtctcctca | |
| 34. | QVQLVESGGG VVQPGRSLRL SCAVSGFTFS SYGMHWVRQA PGKGLEWVAN IWYDGINDYY | Amino Acid |
| | PYSVKGRFTI SRDNSQNTLY LQMNSLRAED TAVYYCARED FINYRSFDYW GQGTLVTVSS | sequence |
| 35. | ggattcacct tcagtagcta tggc | DNA |
| | | sequence |
| 36. | GFTFSSYG | Amino Acid |
| | | sequence |
| 37. | atttggtatg atggaactaa tgat | DNA |
| | | sequence |
| 38. | IWYDGTND | Amino Acid |
| | | sequence |
| 39. | gcgagagagg acttcattaa ctaccggtct tttgactat | DNA |
| | | sequence |
| 40. | AREDFINYRS FDY | Amino Acid |
| | | sequence |
| 41. | caggtgcagc tggtggagtc tgggggaggc gtggtccagc cagggacgtc cctgagactc | DNA |
| | tcctgtgtcg cgtctggatt caccttcaga aattttggaa tgcactgggt ccgccaggct | sequence |
| | ccaggcaagg ggctggagtg ggtggcaaat atatggtttg acggaagtaa tgagaactat | |
| | gtcgagtcca ttcagggccg attcaccatc tccagagaca attccaagaa cacactgaat | |
| | ctgcagatga acagcctgag agccgaggac tcggctgtct attactgtgt gagagaggga | |
| | atcctaggaa ctactaatcc ttatgatgct tttgatgtct ggggccaagg gacaatggtc | |
| | accgtctctt ca | |
| 42. | QVQLVESGGG VVQPGTSLRL SCVASGFTFR NFGMHWVRQA PGKGLEWVAN IWFDGSNENY | Amino Acid |
| | VESIQGRFTI SRDNSKNTLN LQMNSLRAED SAVYYCVREG ILGTINPYDA FDVWGQGTMV | sequence |
| | TVSS | |
| 43. | ggattcacct tcagaaattt tgga | DNA |
| | | sequence |
| 44. | GFTFRNFG | Amino Acid |
| | | sequence |
| 45. | atatggtttg acggaagtaa tgag | DNA |
| | | sequence |
| 46. | IWFDGSNE | Amino Acid |
| | | sequence |
| 47. | gtgagagagg gaatcctagg aactactaat ccttatgatg cttttgatgt c | DNA |
| | | sequence |
| 48. | VREGILGTTN PYDAFDV | Amino Acid |
| | | sequence |
| 49. | caggtgcagc tggtggagtc tgggggaggc gtggtccagc ctgggaggtc cctgagactc | DNA |
| | tcctgtgcag cgtctggatt cacctttagt aactttggaa tgcactgggt ccgccaggcg | sequence |
| | ccaggcaagg gactggagtg ggtggcaggt atatggtttg atggaagtaa taaaaactat | |
| | atagactccg tgaagggccg attcaccatc tcaagagaca attccaagaa cacgctgtat | |
| | ctgcaaatga acagcctgag agccgaggac acggctgtgt attactgtgc gagagagggc | |
| | tatgattcgg ggactgatta tatcccctat gatatttttg atatttgggg ccaagggaca | |
| | atggtcaccg tctcttca | |
| 50. | QVQLVESGGG VVQPGRSLRL SCAASGFTFS NFGMHWVRQA PGKGLEWVAG IWFDGSNKNY | Amino Acid |
| | IDSVKGRFTI SRDNSKNTLY LQMNSLRAED TAVYYCAREG YDSGTDYIPY DIFDIWGQGT | sequence |
| | MVTVSS | |
| 51. | ggattcacct ttagtaactt tgga | DNA |
| | | sequence |
| 52. | GFTFSNFG | Amino Acid |
| | | sequence |
| 53. | atatggtttg atggaagtaa taaa | DNA |
| | | sequence |
| 54. | IWFDGSNK | Amino Acid |
| | | sequence |
| 55. | gcgagagagg gctatgattc ggggactgat tatatcccct atgatatttt tgatatt | DNA |
| | | sequence |
| 56. | AREGYDSGTD YIPYDIFDI | Amino Acid |
| | | sequence |
| 57. | gaagtgcagc tggtggagtc tgggggaggc ttggtacagc ctggcacgtc cctgagactc | DNA |
| | tcctgtgcag cctctggatt cacctttgat gattatgcca tgcactgggt ccggcaagct | sequence+ |
| | ccagggaagg gcctggagtg ggtctcaggt attacttgga atagttataa catagactat | |
| | gctgactctg tgaagggccg attcaccatc tccagagaca acgccaagaa ctccctgtat | |
| | ctgcaaatga acagtctgag agctgaggac acggccttgt attactgtgc aaaagatgat | |
| | gactacagta actacgttta ctttgactac tggggccagg gaaccctggt caccgtctcc | |
| | tca | |
| 58. | EVQLVESGGG LVQPGTSLRL SCAASGFTFD DYAMHWVRQA PGKGLEWVSG ITWNSYNIDY | Amino Acid |
| | ADSVKGRFTI SRDNAKNSLY LQMNSLRAED TALYYCAKDD DYSNYVYFDY WGQGTLVTVS | sequence |
| | S | |
| 59. | ggattcacct ttgatgatta tgcc | DNA |
| | | sequence |
| 60. | GFTFDDYA | Amino Acid |
| | | sequence |
| 61. | attacttgga atagttataa cata | DNA |
| | | sequence |
| 62. | ITWNSYNI | Amino Acid |
| | | sequence |
| 63. | gcaaaagatg atgactacag taactacgtt tactttgact ac | DNA |
| | | sequence |
| 64. | AKDDDYSNYV YFDY | Amino Acid |
| | | sequence |
| 65. | caggttcagc tggtgcagtc cggaactgag gtgaaggagc ctggggcctc agtgaaggtc | DNA |
| | tcctgtaagg cctctggtta ctcctttacc acctatggta tcagctggct gcgacaggcc | sequence |
| | cctggacaag gacttgagtg gatgggatgg atcagcactt acaatggtga cacaatctct | |
| | gcacagatgc tccaggacag agtcaccctg accgcagaca catccacgcg cacagcctac | |
| | atggaactga gaagcctgag atctgacgac acggccgtgt attactgtgc gagaggtcat | |
| | gagtatgata gtcttgttta ttcttactgg ggccagggaa ccctggtcac cgtctcctca | |
| 66. | QVQLVQSGTE VKEPGASVKV SCKASGYSFT TYGISWLRQA PGQGLEWMGW ISTYNGDTIS | Amino Acid |
| | AQMLQDRVTL TADTSTRTAY MELRSLRSDD TAVYYCARGH EYDSLVYSYW GQGTLVTVSS | sequence |
| 67. | ggttactcct ttaccaccta tggt | DNA |
| | | sequence |
| 68. | GYSFTTYG | Amino Acid |
| | | sequence |
| 69. | atcagcactt acaatggtga caca | DNA |
| | | sequence |
| 70. | ISTYNGDT | Amino Acid |
| | | sequence |
| 71. | gcgagaggtc atgagtatga tagtcttgtt tattcttac | DNA |
| | | sequence |
| 72. | ARGHEYDSLV YSY | Amino Acid |
| | | sequence |
| 73. | caggtgcagc tggtggagtc tgggggaggc gtggtccagc ctgggaggtc cctgagactc | DNA |
| | tcctgtgcag cgtctggatt caccttcagt agttatgcca tgcactgggt ccgccaggct | sequence |
| | ccaggcaggg ggctggagtg ggtggcggtt atatggcatg atggagatgt tgaatactat | |
| | gtagactccg tgaaggaccg attcaccatc tccagagaca attccaagag cacgctgtat | |
| | ctgcaaatga acagcctgag agccgaagat acggctttat attattgtgc gagagaggcg | |
| | tgggacctac tacgtccctt tgactattgg ggccagggaa ccctggtcac cgtctcctca | |
| 74. | QVQLVESGGG VVQPGRSLRL SCAASGFTFS SYAMHWVRQA PGRGLEWVAV IWHDGDVEYY | Amino Acid |
| | VDSVKDRFTI SRDNSKSTLY LQMNSLRAED TALYYCAREA WDLLRPFDYW GQGTLVTVSS | sequence |
| 75. | ggattcacct tcagtagtta tgcc | DNA |
| | | sequence |
| 76. | GFTFSSYA | Amino Acid |
| | | sequence |
| 77. | atatggcatg atggagatgt tgaa | DNA |
| | | sequence |
| 78. | IWHDGDVE | Amino Acid |
| | | sequence |
| 79. | gcgagagagg cgtgggacct actacgtccc tttgactat | DNA |
| | | sequence |
| 80. | AREAWDLLRP FDY | Amino Acid |
| | | sequence |
| 81. | gaggtgcagc tggtggagtc tggaggaggc ttggtccagc ctggggggtc cctgagactc | DNA |
| | tcctgtgcag cctctgggtt catcgtcacc accaactaca tgacctggct ccgccaggct | sequence |
| | ccagggaagg ggctggagtg ggtctcactt atttatagca gtggtcacac atactacgca | |
| | gactccgtga agggccgatt caccatctcc agacacaatt ccaagaacac actgtatcta | |
| | caaatggaca gcctgagagc tgaggacacg gccgtgtatt actgtgcgag tgctttcgca | |
| | gcggatgttt ttgatatctg gggccaaggg acaatggtca ccgtctcttc a | |
| 82. | EVQLVESGGG LVQPGGSLRL SCAASGFIVT TNYMTWLRQA PGKGLEWVSL IYSSGHTYYA | Amino Acid |
| | DSVKGRFTIS RHNSKNTLYL QMDSLRAEDT AVYYCASAFA ADVEDIWGQG TMVTVSS | sequence |
| 83. | gggttcatcg tcaccaccaa ctac | DNA |
| | | sequence |
| 84. | GFIVTTNY | Amino Acid |
| | | sequence |
| 85. | atttatagca gtggtcacac a | DNA |
| | | sequence |
| 86. | IYSSGHT | Amino Acid |
| | | sequence |
| 87. | gcgagtgctt tcgcagcgga tgtttttgat atc | DNA |
| | | sequence |
| 88. | ASAFAADVED I | Amino Acid |
| | | sequence |
| 89. | caggtgcagc tggtggagtc tgggggaggc gtggtccagc ctgggaggtc cctgagactc | DNA |
| | tcctgttcag cgtctggatt ctccttcagt cactttggca tgcactgggt ccgccaggtt | sequence |
| | ccaggcgggg gcctggagtg ggtgacaagt atatggtttg atggaagtaa tagatattat | |
| | gcagactcct tgaagggccg attcaccatc tccagagaca attccaagaa tactctgtat | |
| | ctggaaatga acagcctgag agccgaggac acggctgtgt attactgtgt gagagagggg | |
| | atactgggaa ctactaatcc ttatgatgtt tttgatgtct ggggtcaggg gacaatggtc | |
| | accgtctctt ca | |
| 90. | QVQLVESGGG VVQPGRSLRL SCSASGFSFS HFGMHWVRQV PGGGLEWVTS IWFDGSNRYY | Amino Acid |
| | ADSLKGRFTI SRDNSKNTLY LEMNSLRAED TACYYCVREG ILGTTNPYDV FDVWGQGTMV | sequence |
| | TVSS | |
| 91. | ggattctcct tcagtcactt tggc | DNA |
| | | sequence |
| 92. | GFSFSHFG | Amino Acid |
| | | sequence |
| 93. | atatggtttg atggaagtaa taga | DNA |
| | | sequence |
| 94. | IWFDGSNR | Amino Acid |
| | | sequence |
| 95. | gtgagagagg ggatactggg aactactaat ccttatgatg tttttgatgt c | DNA |
| | | sequence |
| 96. | VREGILGTTN PYDVFDV | Amino Acid |
| | | sequence |
| 97. | gaggtgcagc tggtggagtc tgggggaggc ttggtacagc cgggggggtc cctgagactc | DNA |
| | tcctgtgcag cctctggatt cacctttaga agctatgtca tgagctggtt ccgccaggct | sequence |
| | ccagggaagg ggctggagtg ggtctcagga atgagtggga gtggtggaag cacatcctac | |
| | gcagactccg tgaagggccg gttcaccatc tccagagaca attcaaagaa tacgctgtat | |
| | ctgctaatga acagcctgag aaccgaggac acggccgtat attattgtgc gaaagaaaac | |
| | ggggctaact ggaactacgg ctactacggt atggacgtct ggggccaagg gaccacggtc | |
| | accgtctcct ca | |
| 98. | EVQLVESGGG LVQPGGSLRL SCAASGFTFR SYVMSWFRQA PGKGLEWVSG MSGSGGSTSY | Amino Acid |
| | ADSVKGRFTI SRDNSKNTLY LLMNSLRTED TAVYYCAKEN GANWNYGYYG MDVWGQGTTV | sequence |
| | TVSS | |
| 99. | ggattcacct ttagaagcta tgtc | DNA |
| | | sequence |
| 100. | GFTFRSYV | Amino Acid |
| | | sequence |
| 101. | atgagtggga gtggtggaag caca | DNA |
| | | sequence |
| 102. | MSGSGGST | Amino Acid |
| | | sequence |
| 103. | gcgaaagaaa acggggctaa ctggaactac ggctactacg gtatggacgt c | DNA |
| | | sequence |
| 104. | AKENGANWNY GYYGMDV | Amino Acid |
| | | sequence |
| 105. | caggtgcagc tggtggagtc tgggggaggc gtggtccagc ctgggaggtc cctgagactc | DNA |
| | tcctgtgtag cgtctggatt ctccttcagt aactttggca tgcactgggt ccgccaggct | sequence |
| | ccaggcaagg ggctggagtg ggtggcaatt atatggtatg atggaagtaa taaatactat | |
| | tcagactccg tgaagggccg cttcaccatc tccagagaca attccaagaa cacgctgtat | |
| | ctgcaaatga acagcctgag agtcgacgac acggctgtgt attactgtgc gagattcgat | |
| | cgctggaaat ttgacgcttt tgatatctgg ggccaaggga caatggtcac cgtctcttca | |
| 106. | QVQLVESGGG VVQPGRSLRL SCVASGFSFS NFGMHWVRQA PGKGLEWVAI IWYDGSNKYY | Amino Acid |
| | SDSVKGRFTI SRDNSKNTLY LQMNSLRVDD TAVYYCARFD RWKFDAFDIW GQGTMVTVSS | sequence |
| 107. | ggattctcct tcagtaactt tggc | DNA |
| | | sequence |
| 108. | GFSFSNFG | Amino Acid |
| | | sequence |
| 109. | atatggtatg atggaagtaa taaa | DNA |
| | | sequence |
| 110. | IWYDGSNK | Amino Acid |
| | | sequence |
| 111. | gcgagattcg atcgctggaa atttgacgct tttgatatc | DNA |
| | | sequence |
| 112. | ARFDRWKFDA FDI | Amino Acid |
| | | sequence |
| 113. | caggtgcagc tggtggagtc tgggggaggc gtggtccagc ctgggaggtc cctgagactc | DNA |
| | tcctgtgcag cgtctggatt caccttcagt agctttgcca tgcactgggt ccgccaggct | sequence |
| | ccaggcaagg ggctggagtg ggtggcagtt atatggtatg atggaagtaa tgattactat | |
| | gcagcctccg tgaagggccg tttcaccatc tccagagaca attccaagaa cacgctgtat | |
| | ctggaaatga acagactgag agccgaggac acggctgtgt atcactgtgc gagagataac | |
| | tggaattact gggggggtat ggacgtctgg ggccaaggga ccacggtcac cgtctcctca | |
| 114. | QVQLVESGGG VVQPGRSLRL SCAASGFTFS SFAMHWVRQA PGKGLEWVAV IWYDGSNDYY | Amino Acid |
| | AASVKGRFTI SRDNSKNTLY LEMNRLRAED TAVYHCARDN WNYWGGMDVW GQGTTVTVSS | sequence |
| 115. | ggattcacct tcagtagctt tgcc | DNA |
| | | sequence |
| 116. | GFTFSSFA | Amino Acid |
| | | sequence |
| 117. | atatggtatg atggaagtaa tgat | DNA |
| | | sequence |
| 118. | IWYDGSND | Amino Acid |
| | | sequence |
| 119. | gcgagagata actggaatta ctgggggggt atggacgtc | DNA |
| | | sequence |
| 120. | ARDNWNYWGG MDV | Amino Acid |
| | | sequence |
| 121. | gaggtgcagc tgttggagtc tgggggaggc tgggtgcagc cgggggggtc cctgagactc | DNA |
| | tcctgtgcag cctctggatt cgcctttagt aattatgcca tgaactgggt ccgccagact | sequence |
| | ccagggaagg ggctggagtg ggtctcagtt attagtagta gtggtggaaa cacatactac | |
| | gcagactccg tgaagggccg gttcgccatc tccagagaca attccaggga tacgctgcat | |
| | ctgcaaatga acagactgag agtcgaggac acggccgtct attactgtgc gaaagaaata | |
| | cgtccgtatt acgatctttc ctactattac ggtatggacg tctggggcca agggaccacg | |
| | gtcaccgtct cctca | |
| 122. | EVQLLESGGG WVQPGGSLRL SCAASGFAFS NYAMNWVRQT PGKGLEWVSV ISSSGGNTYY | Amino Acid |
| | ADSVKGRFAI SRDNSRDTLH LQMNRLRVED TAVYYCAKEI RPYYDLSYYY GMDVWGQGTT | sequence |
| | VTVSS | |
| 123. | ggattcgcct ttagtaatta tgcc | DNA |
| | | sequence |
| 124. | GFAFSNYA | Amino Acid |
| | | sequence |
| 125. | attagtagta gtggtggaaa caca | DNA |
| | | sequence |
| 126. | ISSSGGNT | Amino Acid |
| | | sequence |
| 127. | gcgaaagaaa tacgtccgta ttacgatctt tcctactatt acggtatgga cgtc | DNA |
| | | sequence |
| 128. | AKEIRPYYDL AYYYGMDV | Amino Acid |
| | | sequence |
| 129. | caggtgcagc tgcaggagtc gggcccagga ctggtgaagt cttcggagac cctgtccctc | DNA |
| | acctgcactg tctctggtgg ctccatcaga aatttctact ggagctggct ccggcagccc | sequence |
| | ccagggaagg gactagagtg gattgggcac atcaattaca atgggggcac cgactacaac | |
| | ccctccctca agagtcgagt caccatatca gtagacacgt ccaagaatca gttctccctg | |
| | aatttgaact ctgtgaccgc cgcagacacg gccgtgtatt actgtgcgag acagagattc | |
| | tacggtatgg acgtctgggg tccagggacc acggtcaccg tctcctca | |
| 130. | QVQLQESGPG LVKKSETLSL TCTVSGGSIR NFYWSWLRQP PGKGLEWIGH INYNGGTDYN | Amino Acid |
| | PSLKSRVTIS VDTSKNQFSL NLNSVTAADT AVYYCARQRF YGMDVWGPGT TVTVSS | sequence |
| 131. | ggtggctcca tcagaaattt ctac | DNA |
| | | sequence |
| 132. | GGSIRNFY | Amino Acid |
| | | sequence |
| 133. | atcaattaca atgggggcac c | DNA |
| | | sequence |
| 134. | INYNGGT | Amino Acid |
| | | sequence |
| 135. | gcgagacaga gattctacgg tatggacgtc | DNA |
| | | sequence |
| 136. | ARQRFYGMDV | Amino Acid |
| | | sequence |
| 137. | gacatccaga tgacccagtc tccatcctcc ctgtctgcat ctgtaggaga cagagtcacc | DNA |
| | atcacttgcc gggcaagtca gagcattagc agctatttaa attggtatca gcagaaacca | sequence |
| | gggaaagccc ctaagctcct gatctatgct gcatccagtt tgcaaagtgg ggtcccgtca | |
| | aggttcagtg gcagtggatc tgggacagat ttcactctca ccatcagcag tctgcaacct | |
| | gaagattttg caacttacta ctgtcaacag agttacagta cccctccgat caccttcggc | |
| | caagggacac gactggagat taaa | |
| 138. | DIQMTQSPSS LSASVGDRVT ITCRASQSIS SYLNWYQQKP GKAPKLLIYA ASSLQSGVPS | Amino Acid |
| | RFSGSGSGTD FTLTISSLQP EDFATYYCQQ SYSTPPITFG QGTRLEIK | sequence |
| 139. | cagagcatta gcagctat | DNA |
| | | sequence |
| 140. | QSISSY | Amino Acid |
| | | sequence |
| 141. | gctgcatcc | DNA |
| | | sequence |
| 142. | AAS | Amino Acid |
| | | sequence |
| 143. | caacagagtt acagtacccc tccgatcacc | DNA |
| | | sequence |
| 144. | QQSYSTPPIT | Amino Acid |
| | | sequence |
| 145. | MKAPAVLAPG ILVLLFTLVQ RSNGECKEAL AKSEMNVNMK YQLPNFTAET PIQNVILHEH | Amino Acid |
| | HIFLGATNYI YVLNEEDLQK VAEYKTGPVL EHPDCFPCQD CSSKANLSGG VWKDNINMAL | sequence |
| | VVDTYYDDQL ISCGSVNRGT CQRHVFPHNH TADIQSEVHC IFSPQIEEPS QCPDCVVSAL | |
| | GAKVLSSVKD RFINFFVGNT INSSYFPDHP LHSISVRRLK ETKDGFMFLT DQSYIDVLPE | |
| | FRDSYPIKYV HAFESNNFIY FLTVQRETLD AQTFHTRIIR FCSINSGLHS YMEMPLECIL | |
| | TEKRKKRSTK KEVFNILQAA YVSKPGAQLS RQIGASLNDD ILFGVFAQSK PDSAEPMDRS | |
| | AMCAFPIKYV NDFFNKIVNK NNVRCLQHFY GPNHEHCFNR TLLRNSSGCE ARRDEYRTEF | |
| | TTALQRVDLF MGQFSEVLLT SISTFIKGDL TIANLGTSEG RFMQVVVSRS GPSTPHVNFL | |
| | LDSHPVSPEV IVEHTLNQNG YTLVITGKKI TKIPLNGLGC RHFQSCSQCL SAPPFVQCGW | |
| | CHDKCVRSEE CLSGTWTQQI CLPAIYKVFP NSAPLEGGTR LTICGWDFGF RRNNKFDLKK | |
| | TRVLLGNESC TLTLSESTMN TLKCTVGPAM NKHFNMSIII SNGHGTTQYS TFSYVDPVIT | |
| | SISPKYGPMA GGTLLTLTGN YLNSGNSRHI SIGGKTCTLK SVSNSILECY TPAQTISTEF | |
| | AVKLKIDLAN RETSIFSYRE DPIVYEIHPT KSFISTWWKE PLNIVSFLFC FASGGSTITG | |
| | VGKNLNSVSV PRMVINVHEA GRNFTVACQH RSNSEIICCT TPSLQQLNLQ LPLKTKAFFM | |
| | LDGILSKYFD LIYVHNPVFK PFEKPVMISM GNENVLEIKG NDIDPEAVKG EVLKVGNKSC | |
| | ENIHLHSEAV LCTVPNDLLK LNSELNIEWK QAISSTVLGK VIVQPDQNFT GLIAGVVSIS | |
| | TALLLLLGFF LWLKKRKQIK DLGSELVRYD ARVHTPHLDR LVSARSVSPT TEMVSNESVD | |
| | YRATFPEDQF PNSSQNGSCR QVQYPLTDMS PILTSGDSDI SSPLLQNTVH IDLSALNPEL | |
| | VQAVQHVVIG PSSLIVHFNE VIGRGHFGCV YHGTLLDNDG KKIHCAVKSL NRITDIGEVS | |
| | QFLTEGIIMK DFSHPNVLSL LGICLRSEGS PLVVLPYMKH GDLRNFIRNE THNPTVKDLI | |
| | GFGLQVAKGM KYLASKKFVH RDLAARNCML DEKFTVKVAD FGLARDMYDK EYYSVHNKTG | |
| | AKLPVKWMAL ESLQTQKFTT KSDVWSFGVL LWELMTRGAP PYPDVNTFDI TVYLLQGRRL | |
| | LQPEYCPDPL YEVMLKCWHP KAEMRPSFSE LVSRISAIFS TFIGEHYVHV NATYVNVKCV | |
| | APYPSLLSSE DNADDEVDTR PASFWETS | |
| 146. | MKAPAVLAPG ILVLLFTLVQ RSNGECKEAL AKSEMNVNMK YQLPNFTAET PIQNVILHEH | Amino Acid |
| | HIFLGATNYI YVLNEEDLQK VAEYKTGPVL EHPDCFPCQD CSSKANLSGG VWKDNINMAL | sequence |
| | VVDTYYDDQL ISCGSVNRGT CQRHVFPHNH TADIQSEVHC IFSPQIEEPS QCPDCVVSAL | |
| | GAKVLSSVKD RFINFFVGNT INSSYFPDHP LHSISVRRLK ETKDGFMFLT DQSYIDVLPE | |
| | FRDSYPIKYV HAFESNNFIY FLTVQRETLD AQTFHTRIIR FCSINSGLHS YMEMPLECIL | |
| | TEKRKKRSTK KEVFNILQAA YVSKPGAQLA RQIGASLNDD ILFGVFAQSK PDSAEPMDRS | |
| | AMCAFPIKYV NDFFNKIVNK NNVRCLQHFY GPNHEHCFNR TLLRNSSGCE ARRDEYRTEF | |
| | TTALQRVDLF MGQFSEVLLT SISTFIKGDL TIANLGTSEG RFMQVVVSRS GPSTPHVNFL | |
| | LDSHPVSPEV IVEHTLNQNG YTLVITGKKI TKIPLNGLGC RHFQSCSQCL SAPPFVQCGW | |
| | CHDKCVRSEE CLSGTWTQQI CLPAIYKVFP NSAPLEGGTR LTICGWDFGF RRNNKFDLKK | |
| | TRVLLGNESC TLTLSESTMN TLKCTVGPAM NKHFNMSIII SNGHGTTQYS TFSYVDPVIT | |
| | SISPKYGPMA GGTLLTLTGN YLNSGNSRHI SIGGKTCTLK SVSNSILECY TPAQTISTEF | |
| | AVKLKIDLAN RETSIFSYRE DPIVYEIHPT KSFISGGSTI TGVGKNLNSV SVPRMVINVH | |
| | EAGRNFTVAC QHRSNSEIIC CTTPSLQQLN LQLPLKTKAF FMLDGILSKY FDLIYVHNPV | |
| | FKPFEKPVMI SMGNENVLEI KGNDIDPEAV KGEVLKVGNK SCENIHLHSE AVLCTVPNDL | |
| | LKLNSELNIE WKQAISSTVL GKVIVQPDQN FTGLIAGVVS ISTALLLLLG FFLWLKKRKQ | |
| | IKDLGSELVR YDARVHTPHL DRLVSARSVS PTTEMVSNES VDYRATFPED QFPNSSQNGS | |
| | CRQVQYPLTD MSPILTSGDS DISSPLLQNT VHIDLSALNP ELVQAVQHVV IGPSSLIVHF | |
| | NEVIGRGHFG CVYHGTLLDN DGKKIHCAVK SLNRITDIGE VSQFLTEGII MKDFSHPNVL | |
| | SLLGICLRSE GSPLVVLPYM KHGDLRNFIR NETHNPTVKD LIGFGLQVAK GMKYLASKKF | |
| | VHRDLAARNC MLDEKFTVKV ADFGLARDMY DKEYYSVHNK TGAKLPVKWM ALESLQTQKF | |
| | TTKSDVWSFC VLLWELMTRC APPYPDVNTF DITVYLLQCR RLLQPEYCPD PLYEVMLKCW | |
| | KPKAEMRPSF SELVSRISAI FSTFIGEHYV HVNATYVNVK CVAPYPSLLS SEDNADDEVD | |
| | TRPASFWETS | |
| 147. | MKAPAVLAPG ILVLLFTLVQ RSNGECKEAL AKSEMNVNMK YQLPNFTAET PIQNVILHEH | Amino Acid |
| | HIFLGATNYI YVLNEEDLQK VARYKTGPVL EHPDCFPCQD CSSKANLSGG VWKDNINMAL | sequence |
| | VVDTYYDDQL ISCGSVNRGT CQRHVFPHNH TADIQSEVHC IFSPQIEEPS QCPDCVVSAL | |
| | GAKVLSSVKD RFINFFVGNT INSSYFPDHP LHSISVRRLK ETKDGFMFLT DQSYIDVLPE | |
| | FRDSYPIKYV HAFESNNFIY FLTVQRETLD AQTFHTRIIR FCSINSGLHS YMEMPLECIL | |
| | TEKRKKRSTK KEVFNILQAA YVSKPGAQLA RQIGASLNDD ILFGVFAQSK PDSAEPMDRS | |
| | AMCAFPIKYV NDFFNKIVNK NNVRCLQHFY GPNHEHCFNR TLLRNSSGCE ARRDEYRTEG | |
| | TTALQRVDLF MGQFSEVLLT SISTFIKGDL TIANLGTSEG RFMQVVVSRS GPSTPHVNFL | |
| | LDSHPVSPEV IVEHTLNQNG YTLVITGKKI TKIPLNGLGC RHFQSCSQCL SAPPFVQCGW | |
| | CHDKCVRSEE CLSGTWTQQI CLPAIYKVFP NSAPLEGGTR LTICGWDFGF RRNNKFDLKK | |
| | TRVLLGNESC TLTLSESTMN TLKCTVGPAM NKHFNMSIII SNGHGTTQYS TFSYVDPVIT | |
| | SISPKYGPMA GGTLLTLTGN YLNSGNSRHI SIGGKTCTLK SVSNSILECY TPAQTISTEF | |
| | AVKLKIDLAN RETSIFSYRE DPIVYEIHPT KSFISGGSTI TGVGKNLNSV SVPRMVINVH | |
| | EAGRNFTVAC QHRSNSEIIC CTTPSLQQLN LQLPLKTKAF FMLDGILSKY FDLIYVHNPV | |
| | FKPFEKPVMI SMGNENVLEA KGNDIDPEAV KGEVLKVGNK SCENIHLHSE AVLCTVPNDL | |
| | LKLNSELNIE VGFLHSSHDV NKEASVIMLF SGLK | |
| 148. | KEALAKSEMN VNMKYQLPNF TAETPIQNVI LHEHHIFLGA TNYIYVLNEE DLQKVAEYKT | Amino Acid |
| | GPVLEHPDCF PCQDCSSKAN LSGGVWKDNI NMALVVDTYY DDQLISCGSV SRGTCQRHVF | sequence |
| | PHNHTADIQS EVHCIFSPQI EEPSQCPDCV VSALGAKVLS SVKDRFINFF VGNTINSSYF | |
| | PDHPLHSISV RRLKETKDGF MFLTDQSYID VLPEFRDSYP IKYVHAFESN NFIYFLTVQR | |
| | ETLDAQTFHT RIIRFCSINS GLHSYMEMPL ECILTEKRKK R | |
| 149. | STKKEVFNIL QAAYVSKPGA QLARQIGASL NDDILFGVFA QSKPDSAEPM DRSAMCAFPI | Amino Acid |
| | KYVNDFFNKI VNKNNVRCLQ HFYGPNHEHC FNRTLLRNSS GCEARRDEYR TEFTTALQRV | sequence |
| | DLFMGQFSEV LLTSISTFIK GDLTIANLGT SEGRFMQVVV SRSGPSTPHV NFLLDSHPVS | |
| | PEVIVEHTLN QNGYTLVITG KKITKIPLNG LGCRHFQSCS QCLSAPPFVQ CGWCHDKCVR | |
| | SEECLSGTWT QQICLPAIYK VFPNSAPLEG GTRLTICGWD FGFRRNNKFD LKKTRVLLGN | |
| | ESCTLTLSES TMNTLKCTVG PAMNKHFNMS IIISNGHGTT QYSTFSYVDP VITSISPKYG | |
| | PMAGGTLLTL TGNYLNSGNS RHISIGGKTC TLKSVSNSIL ECYTPAQTIS TEFAVKLKID | |
| | LANRETSIFS YREDPIVYEI HPTKSFISTW WKEPLNIVSF LFCFASGGST ITGVGKNLNS | |
| | VSVPRMVINV HEAGRNFTVA CQHRSNSEII CCTTPSLQQL NLQLPLKTKA FFMLDGILKS | |
| | YFDLIYVHNP VFKPFEKPVM ISMGNENVLE IKGNDIDPEA VKGEVLKVGN KSCENIHLHS | |
| | EAVLCTVPND LLKLNSELNI EWKQAISSTV LGKVIVQPDQ NFTGLIAGVV SISTALLLLL | |
| | GFFLWLKKRK QIKDLGSELV RYDARVHTPH LDRLVSARSV SPTTEMVSNE SVDYRATFPE | |
| | DQFPNSSQNG SCRQVQYPLT DMSPILTSGD SDISSPLLQN TVHIDLSALN PELVQAVQHV | |
| | VIGPSSLIVH FNEVIGRGHF GCVYHGTLLD NDGKKIHCAV KSLNRITDIG EVSQFLTEGI | |
| | IMKDFSHPNV LSLLGICLRS EGSPLVVLPY MKHGDLRNFI RNETHNPTVK DLIGFGLQVA | |
| | KGMKYLASKK FVHRDLAARN CMLDEKFTVK VADFGLARDM YDKEYYSVHN KTGAKLPVKW | |
| | MALESLQTQK FTTKSDVWSF GVLLWELMTR GAPPYPDVNT FDITVYLLQG RRLLQPEYCP | |
| | DPLYEVMLKC WHPKAEMRPS FSELVSRISA IFSTFIGEHY VHVNATYVNV KCVAPYPSLL | |
| | SSEDNADDEV DTRPASFWET S | |
| 150. | TKKEVFNILQ AAYVSKPGAQ LARQIGASLN DDILFGVFAQ SKPDSAEPMD RSAMCAFPIK | Amino Acid |
| | YVNDFFNKIV NKNNVRCLQH FYGPNHEHCF NRTLLRNSSG CEARRDEYRT EFTTALQRVD | sequence |
| | LFMGQFSEVL LTSISTFIKG DLTIANLGTS EGRFMQVVVS RSGPSTPHVN FLLDSHPVSP | |
| | EVIVEHTLNQ NGYTLVITGK KITKIPLNGL GCRHFQSCSQ CLSAPPFVQC GWCHDKCVRS | |
| | EECLSGTWTQ QICLPAIYKV FPNSAPLEGG TRLTICGWDF GFRRNNKFDL KKTRVLLGNE | |
| | SCTLTLSEST MNTLKCTVGP AMNKHFNMSI IISNGHGTTQ YSTFSYVDPV ITSISPKYGP | |
| | MAGGTLLTLT GNYLNSGNSR HISIGGKTCT LKSVSNSILE CYTPAQTIST EFAVKLKIDL | |
| | ANRETSIFSY REDPIVYEIH PTKSFISGGS TITGVGKNLN SVSVPRMVIN VHEAGRNFTV | |
| | ACQHRSNSEI ICCTTPSLQQ LNLQLPLKTK AFFMLDGILS KYFDLIYVHN PVFKPFEKPV | |
| | MISMGNENVL EIKGNDIDPE AVKGEVLKVG NKSCENIHLH SEAVLCTVPN DLLKLNSELN | |
| | IEWKQAISST VLGKVIVQPD QNFTGLIAGV VSISTALLLL LGFFLWLKKR KQIKDLGSEL | |
| | VRYDARVHTP HLDRLVSARS VSPTTEMVSN ESVDYRATFP EDQFPNSSQN GSCRQVQYPL | |
| | TDMSPILTSG DSDISSPLLQ NTVHIDLSAL NPELVQAVQH VVIGPSSLIV HFNEVIGRGH | |
| | FGCVYHGTLL DNDGKKIHCA VKSLNRITDI GEVSQFLTEG IIMKDFSHPN VLSLLGICLR | |
| | SEGSPLVVLP YMKHGDLRNF IRNETHNPTV KDLIGFGLQV AKGMKYLASK KFVHRDLAAR | |
| | NCMLDEKFTV KVADFGLARD MYDKEYYSVH NKTGAKLPVK WMALESLQTQ KFTTKSDVWS | |
| | FGVLLWELMT RGAPPYPDVN TFDITVYLLQ GRRLLQPEYC PDPLYEVMLK CWHPKAEMRP | |
| | SFSELVSRIS AIFSTFIGEH YVHVNATYVN VKCVAPYPSL LSSEDNADDE VDTRPASFWE | |
| | TS | |
| 151. | TKKEVFNILQ AAYVSKPGAQ LARQIGASLN DDILFGVFAQ SKPDSAEPMD RSAMCAFPIK | Amino Acid |
| | YVNDFFNKIV NKNNVRCLQH FYGPNHEHCF NRTLLRNSSG CEARRDEYRT EFTTALQRVD | sequence |
| | LFMGQFSEVL LTSISTFIKG SLTIANLGTS EGRFMQVVVS RSGPSTPHVN FLLDSHPVSP | |
| | EVIVEHTLNQ GNYTLVITGK KITKIPLNGL GCRHFQSCSQ CLSAPPFVQC GWCHDKCVRS | |
| | EECLSGTWTQ QICLPAIYKV FPNSAPLEGG TRLTICGWDF GFRRNNKFDL KKTRVLLGNE | |
| | SCTLTLSEST MNTLKCTVGP AMNKHFNMSI IISNGHGTTQ YSTFSYVDPV ITSISPKYGP | |
| | MAGGTLLTLT GNYLNSGNSR HISIGGKTCT LKSVSNSILE CYTPAQTIST EFAVKLKIDL | |
| | ANRETSIFSY REDPIVYEIH PTKSFISGGS TITGVGKNLN SVSVPRMVIN VHEAGRNFTV | |
| | ACQHRSNSEI ICCTTPSLQQ LNLQLPLKTK AFFMLDGILS KYFDLIYVHN PVFKPFEKPV | |
| | MISMGNENVL EIKGNDIDPE AVKGEVLKVG NKSCENIHLH SEAVLCTVPN DLLKLNSELN | |
| | IEVGFLHSSH DVNKEASVIM LFSGLK | |
| 152. | TKKEVFNILQ AAYVSKPGAQ LARQIGASLN DDILFGVFAQ SKPDSAEPMD RSAMCAFPIK | Amino Acid |
| | YVNDFFNKIV NKNNVRCLQH FYGPNHEHCF NRTLLRNSSG CEARRDEYRT EFTTALQRVD | sequence |
| | LFMGQFSEVL LTSISTFIKG DLTIANLGTS EGRFMQVVVS RSGPSTPHVN FLLDSHPVSP | |
| | EVIVEHTLNQ NGYTLVITGK KITKIPLNGL GCRHFQSCSQ CLSAPPFVQC GWCHDKCVRS | |
| | EECLSGTWTQ QICLPAIYKV FPNSAPLEGG TRLTICGWDF GFRRNNKFDL KKTRVLLGNE | |
| | SCTLTLSEST MNTLKCTVGP AMNKHFNMSI IISNGHGTTQ YSTFSYVDPV ITSISPKYGP | |
| | MAGGTLLTLT GNYLNSGNSR HISIGGKTCT LKSVSNSILE CYTPAQTIST EFAVKLKIDL | |
| | ANRETSIFSY REDPIVYEIH PTKSFISTWW KEPLNIVSFL FCFASGGSTI TGVGKNLNSV | |
| | SVPRMVINVH EAGRNFTVAC QHRSNSEIIC CTTPSLQQLN LQLPLKTKAF FMLDGILSKY | |
| | FDLIYVHNPV FKPFEKPVMI SMGNENVLEI KGNDIDPEAV KGEVLKVGNK SCENIHLHSE | |
| | AVLCTVPNDL LKLNSELNIE WKQAEQKLIS EEDLEQKLIS EEDLHHHHHH | |
| 153. | TKKEVFNILQ AAYVSKPGAQ LARQIGASLN DDILFGVFAQ SKPDSAEPMD RSAMCAFPIK | Amino Acid |
| | YVNDFFNKIV NKNNVRCLQH FYGPNHEHCF NRTLLRNSSG CEARRDEYRT EFTTALQRVD | sequence |
| | LFMGQFSEVL LTSISTFIKG DLTIANLGTS EGRFMQVVVS RSGPSTPHVN FLLDSHPVSP | |
| | EVIVEHTLNQ NGYTLVITGK KITKIPLNGL GCRHFQSCSQ CLSAPPFVQC GWCHDKCVRS | |
| | EECLSGTWTQ QICLPAIYKV FPNASPLEGG TRLTICGWDF GFRRNNKFDL KKTRVLLGNE | |
| | SCTLTLSEST MNTLKCTVGP AMNKHFNMSI IISNGHGTTQ YSTFSYVDPV ITSISPKYGP | |
| | MAGGTLLTLT GNYLNSGNSR HISIGGKTCT LKSVSNSILE CYTPAQTIST EFAVKLKIDL | |
| | ANRETSIFSY REDPIVYEIH PTKSFISTWW KEPLNIVSFL FCFASGGSTI TGVGKNLNSV | |
| | SCPRMVINVH EAGRNFTVAC QHRSNSEIIC CTTPSLQQLN LQLPLKTKAF FMLDGILSKY | |
| | FDLIYVHNPV FKPFEKPVMI SMGNENVLEI KGNDIDPEAV KGEVLKVGNK SCENIHLHSE | |
| | ACLCTVPNDL LKLNSELNIE WKQAMEVSSV FIFPPKPKDV LTITLTPKVT CVVVDISKDD | |
| | PEVQFSWFVD DVEVHTAQTQ PREEQFNSTF RSVSELPIMH QDWLNGKEFK CRVNSAAFPA | |
| | PIEKTISKTK GRPKAPQVYT IPPPKEQMAK DKVSLTCMIT DFFPEDITVE WQWNGQPAEN | |
| | YKNTQPIMDT DGSYFVYSKL NVQKSNWEAG NTFTCSVLHE GLHNHHTEKS LSHSPGKE | |
| 154. | STKKEVFNIL QAAYVSKPGA QLARQIGASL NDDILFGVFA QSKPDSAEPM DRSAMCAFPI | Amino Acid |
| | KYVNDFFNKI VNKNNVRCLQ HFYGPNHEHC FNRTLLRNSS GCEARRDEYR AEFTTALQRV | sequence |
| | DLFMGQFSEV LLTSISTFVK GDLTIANLGT SEGRFMQVVV SRSGPSTPHV NFLLDSHPVS | |
| | PEVIVEHPLN QNGYTLVVTG KKITKIPLNG LGCRHFQSCS QCLSAPPFVQ CGWCHDKCVR | |
| | SEECPSGTWT QQICLPAIYK VFPTSAPLET GTRLTICGWD FGFRRNNKFD LKKTRVLLGN | |
| | ESCTLTLSES TMNTLKCTVG PAMNKHFNMS IIISNGHGTT QYSTFSYVDP IITSISPKYG | |
| | PMAGGTLLTL TGNYLNSGNS RHISIGGKTC TLKSVSNSIL ECYTPAQTIS TEFAVKLKID | |
| | LANRETSIFS YREDPIVYEI HPTKSFISGG STITGVGKNL HSVSVPRMVI NVHEAGRNFT | |
| | VACQHRSNSE IICCTTPSLQ QLNLQLPLKT KAFFMLDGIL SKYFDLIYVH NPVFKPFEKP | |
| | VMISMGNENV LEIKGNDIDP EAVKGEVLKV GNKSCENIHL HSEAVLCTVP NDLLKLNSEL | |
| | NIEWKQAISS TVLGKVIVQP DQNFTGLIAG VVSISIALLL LLGLFLWLKK RKQIKDLGSE | |
| | LVRYDARVHT PHLDRLVSAR SVSPTTEMVS NESVDYRATF PEDQFPNSSQ NGSCRQVQYP | |
| | LTDMSPILTS GDSDISSPLL QNTVHIDLSA LNPELVQAVQ HVVIGPSSLI VHFNEVIGRG | |
| | HFGCVYHGTL LDNDGKKIHC AVKSLNRITD IGEVSQFLTE GIIMKDFSHP NVLSLLGICL | |
| | RSEGSPLVVL PYMKHGDLRN FIRNETHNPT VKDLIGFGLQ VAKGMKYLAS KKFVHRDLAA | |
| | RNCMLDEKFT VKVADFGLAR DMYDKEYYSV HNKTGAKLPV KWMALESLQT QKFTTKSDVW | |
| | SFGVLLWELM TRGAPPYPDV NTFDITVYLL QGRRLLQPEY CPDPLYEVML KCWHPKAEMR | |
| | PSFSELVSRI SAIFSTFIGE HYVHVNATYV NVKCVAPYPS LLSSEDNADD EVDTEQKLIS | |
| | EEDLEQKLIS EEDLHHHHHH | |
| 155. | ECKEALAKSE MNVNMKYQLP MFTAETPIQN VILHEHHIFL GATNYIYVLN EEDLQKVAEY | Amino Acid |
| | KTGPVLEHPD CFPCQDCSSK ANLSGGVWKD NINMALVVDT YYDDQLISCG SVNRGTCQRH | sequence |
| | VFPHNHTADI QDEVHCIFSP QIEEPSQCPD CVVSALGAKV LSSVKDRFIN FFVGNTINSS | |
| | YFPDHPLHSI SVRRLKETKD GFMFLTDQSY IDVLPEFRDS YPIKYVHAFE SNNFIYFLTV | |
| | QRETLDAQTF HTRIIRFCSI NSGLHSYMEM PLECILTEKR KKRSTKKEVF NILQAAYVSK | |
| | PGAQLARQIG ASLNDDILFG VFAQSKPDSA EPMDRSAMCA FPIKYVNDFF NKIVNKNNVR | |
| | CLQHFYGPNH EHCFNRTLLR NSSGCEARRD EYRTEFTTAL QRVDLFMGQF SEVLLTSIST | |
| | FIKGDLTIAN LGTSEGRFMQ VVVSRSGPST PHVNFLLDSH PVSPEVIVEH TLNQNGYTLV | |
| | ITGKKITKIP LNGLGCRHFQ SCSQCLSAPP FVQCGWCHDK CVRSEECLSG TWTQIICLPA | |
| | IYKVFPNSAP LEGGTRLTIC GWDFGFRRNN KFDLKKTRVL LGNESCTLTL SESTMNTLKC | |
| | TVPGAMNKHF NMSIIISNGH GTTQYSTFSY VDPVITSISP KYGPMAGGTL LTLTGNYLNS | |
| | GNSHRISIGG KTCTLKSVSN SILECYTPAQ TISTEFAVKL KIDLANRETS IFSYREDPIV | |
| | YEIHPTKSFI SGGSTITGVG KNLNSVSVPR MVINVHEAGR NFTVACQHRS NSEIICCTTP | |
| | SLQQLNLQLP LKTKAFFMLD GILSKYFDLI YVHNPVFKPF EKPVMISMGN ENVLEIKGND | |
| | IDPEAVKGEV LKVGNKSCEN IHLHSEAVLC TVPNDLLKLN SELNIEWKQA ISSTVLGKVI | |
| | VQPDQNFTEQ KLISEEDLGG EQKLISEEDL HHHHHH | |
| 156. | VRRLKETKDG FM | Amino Acid |
| | | sequence |
| 157. | LARQIGASLN D | Amino Acid |
| | | sequence |
| 158. | FIKGDLTIAN LGTSEGRFMQ VVVSRSGPST PHVNF | Amino Acid |
| | | sequence |

What is claimed is:

1.-38. (canceled)

39. A method for monitoring the efficacy of an anti-cancer therapy in a subject, the method comprising:
(a) administering a radiolabeled antibody conjugate to the subject wherein the subject has received the anti-cancer therapy; and
(b) imaging localization of the radiolabeled antibody conjugate in the cancer by positron emission tomography (PET) imaging,
wherein a decrease in the baseline in the area of localization of the radiolabeled antibody conjugate indicates cancer regression and efficacy of the anti-cancer therapy; and
wherein the radiolabeled antibody conjugate comprises an antibody or antigen-binding fragment thereof that binds MET, a chelating moiety, and a positron emitter, and wherein the antibody or antigen-binding fragment thereof has one or more properties selected from the group consisting of:
(i) comprises a heavy chain variable region (HCVR) having an amino acid sequence selected from the group consisting of SEQ ID NOs: 2, 10, 18, 26, 34, 42, 50, 58, 66, 74, 82, 90, 98, 106, 114, 122, and 130, or a substantially similar sequence thereof having at least 90%, at least 95%, at least 98% or at least 99% sequence identity;
(ii) comprises a light chain variable region (LCVR) having an amino acid sequence of SEQ ID NO: 138, or a substantially similar sequence thereof having at least 90%, at least 95%, at least 98% or at least 99% sequence identity;
(iii) comprises a heavy chain complementarity determining region (HCDR)-3 domain having an amino acid sequence selected from the group consisting of SEQ ID NOs: 8, 16, 24, 32, 40, 48, 56, 64, 72, 80, 88, 96, 104, 112, 120, 128 and 136, or a substantially similar sequence thereof having at least 90%, at least 95%, at least 98% or at least 99% sequence identity; and a light chain complementarity determining region (LCDR)-3 domain having an amino acid sequence of SEQ ID NO: 144, or a substantially similar sequence thereof having at least 90%, at least 95%, at least 98% or at least 99% sequence identity;
(iv) comprises an HCDR1 domain having an amino acid sequence selected from the group consisting of SEQ ID NOs: 4, 12, 20, 28, 36, 44, 52, 60, 68, 76, 84, 92, 100, 108, 116, 124, and 132, or a substantially similar sequence thereof having at least 90%, at least 95%, at least 98% or at least 99% sequence identity; an HCDR2 domain having an amino acid sequence selected from the group consisting of SEQ ID NOs: 6, 14, 22, 30, 38, 46, 54, 62, 70, 78, 86, 94, 102, 110, 118, 126, and 134, or a substantially similar sequence thereof having at least 90%, at least 95%, at least 98% or at least 99% sequence identity; an LCDR1 domain having an amino acid sequence of SEQ ID NO: 140, or a substantially similar sequence thereof having at least 90%, at least 95%, at least 98% or at least 99% sequence identity; and an LCDR2 domain having an amino acid sequence of SEQ ID NO: 142, or a substantially similar sequence thereof having at least 90%, at least 95%, at least 98% or at least 99% sequence identity;
(v) is a multi-specific antigen-binding molecule comprising a first binding specificity to MET and a second binding specificity to a tumor specific antigen;
(vi) is a multi-specific antigen-binding molecule comprising a first binding specificity to one epitope of MET and a second binding specificity to a second epitope of MET;
(vii) binds to monomeric human MET (e.g., hMET.mmh) with a binding dissociation equilibrium constant (K_{D}) of less than about 230 nM as measured by surface plasmon resonance at 25° C. or 37° C.;
(viii) binds to dimeric human MET with a K_{D} of less than about 3 nM as measured by surface plasmon resonance at 25° C. or 37° C.;
(ix) blocks the binding of HGF to MET; and
(x) suppresses cancer growth and increases survival in subjects with cancer.

40. The method of claim 39, wherein the subject is administered 0.1-10 mg/kg of the radiolabeled antibody conjugate.

41. The method of claim 39, wherein the radiolabeled antibody conjugate is administered subcutaneously or intravenously to the subject.

42. The method of claim 39, wherein PET imaging is done 2-7 days after administering the radiolabeled antibody conjugate.

43. The method of claim 39, wherein the anti-cancer therapy is an inhibitor of the HGF/MET signaling pathway.

44. The method of claim 43, wherein the subject is administered the radiolabeled antibody conjugate 1-20 weeks after administration of the inhibitor of the HGF/MET signaling pathway.

45. The method of claim 39, wherein the cancer is selected from the group consisting of acute myelogenous leukemia, adult T-cell leukemia, astrocytoma, bladder cancer, breast cancer, cervical cancer, cholangiocarcinoma, chronic myeloid leukemia, colorectal cancer, endometrial cancer, esophageal cancer, gastric cancer (e.g., gastric cancer with MET amplification), glioblastoma, head and neck cancer (e.g., head and neck squamous cell carcinoma [HNSCC]), Kaposi's sarcoma, kidney cancer, leiomyosarcoma, liver cancer, lung cancer (e.g., non-small cell lung cancer [NSCLC]), lymphoma, malignant glioma, malignant mesothelioma, melanoma, mesothelioma, MFH/fibrosarcoma, multiple myeloma, nasopharyngeal cancer, osteosarcoma, ovarian cancer, pancreatic carcinoma, prostate cancer, renal cell carcinoma, rhabdomyosarcoma, small cell lung cancer, synovial sarcoma, thyroid cancer, and Wilms' tumor.

46. The method of claim 45, wherein the cancer is selected from the group consisting of gastric cancer and non-small cell lung cancer.

47. The method of claim 39, wherein the antibody or antigen-binding fragment thereof is covalently bonded to one or more moieties of Formula (A): 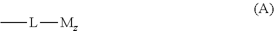
wherein L is the chelating moiety; M is the positron emitter; and z, independently at each occurrence, is 0 or 1; and wherein at least one of z is 1.

48. The method of claim 39, wherein the chelating moiety comprises desferrioxamine.

49. The method of claim 39, wherein the positron emitter is ⁸⁹Zr.

50. The method of claim 47, wherein-L-M is: 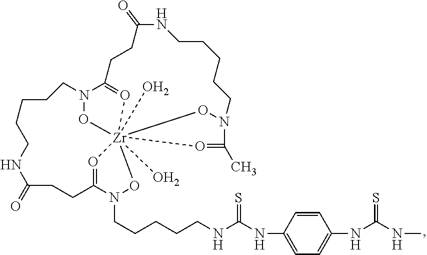
wherein Zr is the positron emitter ⁸⁹Zr.

51. The method of claim 47, wherein the antibody or antigen-binding fragment thereof is covalently bonded to one, two, or three moieties of Formula (A).

52. The method of claim 39, wherein the antibody or antigen-binding fragment thereof comprises three heavy chain complementarity determining regions (HCDRs) in a heavy chain variable region (HCVR), wherein the HCVR has an amino acid sequence selected from the group consisting of SEQ ID NOs: 2, 10, 18, 26, 34, 42, 50, 58, 66, 74, 82, 90, 98, 106, 114, 122, and 130; and three light chain complementarity determining regions (LCDRs) in a light chain variable region (LCVR), wherein the LCVR has an amino acid sequence of SEQ ID NOs: 138.

53. The method of claim 39, wherein the antibody or antigen-binding fragment thereof comprises three CDRs in an HCVR of SEQ ID NO: 18.

54. The method of claim 39, wherein the antibody or antigen-binding fragment thereof comprises three CDRs in an HCVR of SEQ ID NO: 58.

55. The method of claim 39, wherein the antibody or antigen-binding fragment thereof comprises three CDRs in an HCVR of SEQ ID NO: 82.

56. The method of claim 39, wherein the antibody or antigen-binding fragment thereof comprises three CDRs in an LCVR of SEQ ID NO: 138.

57. The method of claim 39, wherein the antibody or antigen-binding fragment thereof comprises an HCDR1 as set forth in SEQ ID NO: 20, an HCDR2 as set forth in SEQ ID NO: 22, and an HCDR3 as set forth in SEQ ID NO: 24.

58. The method of claim 39, wherein the antibody or antigen-binding fragment thereof comprises an HCDR1 as set forth in SEQ ID NO: 60, an HCDR2 as set forth in SEQ ID NO: 62, and an HCDR3 as set forth in SEQ ID NO: 64.

59. The method of claim 39, wherein the antibody or antigen-binding fragment thereof comprises an HCDR1 as set forth in SEQ ID NO: 84, an HCDR2 as set forth in SEQ ID NO: 86, and an HCDR3 as set forth in SEQ ID NO: 88.

60. The method of claim 39, wherein the antibody or antigen-binding fragment thereof comprises an LCDR1 as set forth in SEQ ID NO: 140, an LCDR2 as set forth in SEQ ID NO:
142, and an LCDR3 as set forth in SEQ ID NO: 144.

61. The method of claim 39, wherein the antibody or antigen-binding fragment thereof comprises:
(i) a first antigen-binding domain (D1); and
(ii) a second antigen-binding domain (D2);
wherein D1 specifically binds a first epitope of human MET; and
wherein D2 specifically binds a second epitope of human MET.

62. The method of claim 61, wherein:
(i) D1 comprises the CDRs within an HCVR amino acid sequence of SEQ ID NO: 58; and
(ii) D2 comprises the CDRs within an HCVR amino acid sequence of SEQ ID NO: 82.

63. The method of claim 61, wherein:
(i) D1 comprises the CDRs within an LCVR amino acid sequence of SEQ ID NO: 138; and
(ii) D2 comprises the CDRs within an LCVR amino acid sequence of SEQ ID NO: 138.

64. The radiolabeled antibody conjugate of claim 61, wherein:
(i) D1 comprises a HCDR1-HCDR2-HCDR3-LCDR1-LCDR2-LCDR3 set of amino acid sequences of SEQ ID NOs: 60-62-64-140-142-144; and
(ii) D2 comprises a HCDR1-HCDR2-HCDR3-LCDR1-LCDR2-LCDR3 set of amino acid sequences of SEQ ID NOs: 84-86-88-140-142-144.

65. The method of claim 43, wherein the inhibitor of the HGF/MET signaling pathway is an antibody or antigen-binding fragment thereof.

66. The method of claim 43, wherein the inhibitor of the HGF/MET signaling pathway is a MET×MET bispecific antibody or antigen-binding fragment thereof.

67. The method of claim 66, wherein the MET×MET bispecific antibody or antigen-binding fragment thereof comprises:
(i) a first antigen-binding domain (D1) comprising the CDRs within an HCVR amino acid sequence of SEQ ID NO: 58; and
(ii) a second antigen-binding domain (D2) comprising the CDRs within an HCVR amino acid sequence of SEQ ID NOs: 82.

68. The method of claim 67, wherein the MET×MET bispecific antibody or antigen-binding fragment thereof comprises the CDRs within an LCVR amino acid sequence of SEQ ID NO: 138.

69. The method of claim 67, wherein:
(i) D1 comprises an HCVR of SEQ ID NO: 58; and
(ii) D2 comprises an HCVR of amino acid sequence SEQ ID NO: 82.

70. A method for predicting a positive response to an anti-cancer therapy in a subject, the method comprising:
(a) administering a radiolabeled antibody conjugate to the subject wherein the subject has received the anti-cancer therapy; and
(b) imaging localization of the radiolabeled antibody conjugate in the cancer by positron emission tomography (PET) imaging,
wherein the presence of the radiolabeled antibody conjugate in the cancer indicates that the cancer is MET positive,
wherein a MET positive cancer predicts a positive response to the anti-cancer therapy.

71. The method of claim 70, wherein the radiolabeled antibody conjugate comprises an antibody or antigen-binding fragment thereof that binds MET, a chelating moiety, and a positron emitter,
wherein the antibody or antigen-binding fragment thereof is a MET×MET bispecific antibody or antigen-binding fragment thereof and comprises:
(i) a first antigen-binding domain (D1) comprising the CDRs within an HCVR amino acid sequence of SEQ ID NO: 58;
(ii) a second antigen-binding domain (D2) comprising the CDRs within an HCVR amino acid sequence of SEQ ID NO: 82.

72. The method of claim 71, wherein the MET×MET bispecific antibody or antigen-binding fragment thereof comprises the CDRs within an LCVR amino acid sequence of SEQ ID NO: 138.

73. A method for predicting efficacy of an anti-cancer therapy in a subject, the method comprising:
(a) determining the level of MET expression in the cancer comprising administering a radiolabeled conjugate to the cancer, and visualizing MET expression by PET imaging, wherein the presence of the radiolabeled antibody conjugate in the cancer indicates that the cancer is MET positive; and
(b) correlating the level of MET expression with successful anti-cancer therapy,
wherein a decrease in MET expression, relative to MET expression prior to anti-cancer therapy, is predictive of successful anti-cancer therapy.

74. The method of claim 73, wherein the radiolabeled antibody conjugate comprises an antibody or antigen-binding fragment thereof that binds MET, a chelating moiety, and a positron emitter,
wherein the antibody or antigen-binding fragment thereof is a MET×MET bispecific antibody or antigen-binding fragment thereof and comprises:
(i) a first antigen-binding domain (D1) comprising the CDRs within an HCVR amino acid sequence of SEQ ID NO: 58;
(ii) a second antigen-binding domain (D2) comprising the CDRs within an HCVR amino acid sequence of SEQ ID NO: 82.

75. The method of claim 74, wherein the MET×MET bispecific antibody or antigen-binding fragment thereof comprises the CDRs within an LCVR amino acid sequence of SEQ ID NO: 138.


SEQUENCE LISTING (158 sequences)

SEQ ID NO: 1 - DNA, 372 bp, Homo sapiens
  source 1..372: mol_type = other DNA
caggtgcagctgcaggagtcgggcccaggactggtgaagccttcggagaccctgtccctcacctgcactgtctctggtgactccatcagtagttactattggacctggatccggcagcccccagggaagggactggagtggattgggtatatcttttacagggggggcaccacctacaacccctccctcaagagtcgagtcaccatatcagtagacacgtccaagaaccagttctccctgaagttgaggtctgtgaccgccgcagacacggccgtgtattactgtgcgaggggagacgatcttttagtggtgacaagtgtctactggtacatcgatctctggggccgtggcaccctggtcaccgtctcctca

SEQ ID NO: 2 - protein, 124 aa, Homo sapiens
QVQLQESGPGLVKPSETLSLTCTVSGDSISSYYWTWIRQPPGKGLEWIGYIFYRGGTTYNPSLKSRVTISVDTSKNQFSLKLRSVTAADTAVYYCARGDDLLVVTSVYWYIDLWGRGTLVTVSS

SEQ ID NO: 3 - DNA, 24 bp, Homo sapiens
  source 1..24: mol_type = other DNA
ggtgactccatcagtagttactat

SEQ ID NO: 4 - protein, 8 aa, Homo sapiens
GDSISSYY

SEQ ID NO: 5 - DNA, 21 bp, Homo sapiens
  source 1..21: mol_type = other DNA
atcttttacagggggggcacc

SEQ ID NO: 6 - protein, 7 aa, Homo sapiens
IFYRGGT

SEQ ID NO: 7 - DNA, 54 bp, Homo sapiens
  source 1..54: mol_type = other DNA
gcgaggggagacgatcttttagtggtgacaagtgtctactggtacatcgatctc

SEQ ID NO: 8 - protein, 18 aa, Homo sapiens
ARGDDLLVVTSVYWYIDL

SEQ ID NO: 9 - DNA, 360 bp, Homo sapiens
  source 1..360: mol_type = other DNA
caggtgcagctggtggagtctgggggaggcgtggtccagcctggggggtccctgagactctcctgtgcagcgtccggattcaccttcagtggctatggcatgcactgggtccgccaggctccaggcaaggggctggagtggatggcagttatatggtatgatggaagtaatgattactatccagactccgtgaagggccgattcaccatctccagagacaattccaagaacacgctgtatctgcaaatgaacagcctgagagtcgaggacacggctgtgtattactgtgcgcgagatgcgtgggacctactacgttcctttgactactggggccagggaaccctggtcaccgtctcctca

SEQ ID NO: 10 - protein, 120 aa, Homo sapiens
QVQLVESGGGVVQPGGSLRLSCAASGFTFSGYGMHWVRQAPGKGLEWMAVIWYDGSNDYYPDSVKGRFTISRDNSKNTLYLQMNSLRVEDTAVYYCARDAWDLLRSFDYWGQGTLVTVSS

SEQ ID NO: 11 - DNA, 24 bp, Homo sapiens
  source 1..24: mol_type = other DNA
ggattcaccttcagtggctatggc

SEQ ID NO: 12 - protein, 8 aa, Homo sapiens
GFTFSGYG

SEQ ID NO: 13 - DNA, 24 bp, Homo sapiens
  source 1..24: mol_type = other DNA
atatggtatgatggaagtaatgat

SEQ ID NO: 14 - protein, 8 aa, Homo sapiens
IWYDGSND

SEQ ID NO: 15 - DNA, 39 bp, Homo sapiens
  source 1..39: mol_type = other DNA
gcgcgagatgcgtgggacctactacgttcctttgactac

SEQ ID NO: 16 - protein, 13 aa, Homo sapiens
ARDAWDLLRSFDY

SEQ ID NO: 17 - DNA, 363 bp, Homo sapiens
  source 1..363: mol_type = other DNA
caggtgcagctggtggagtctgggggaggcgtggtccagcctgggaggtccctgagagtctcttgtgtagtgtctggattcaccttcagcagctttggcatgcattgggtccgccaggctccagacaaggggctggagtgggtggcagttatatggtatgatggaagtaatgattactattcagactccgtgaagggccgattcaccatctccagagacaattccaagaacacgctgtttctacaaatgaaccgcctgagagccgaagacacggctgtttattactgtgcgcgagctaataactggaaccgttttgatgcctttgatctctggggccaagggacaatggtcaccgtctcttca

SEQ ID NO: 18 - protein, 121 aa, Homo sapiens
QVQLVESGGGVVQPGRSLRVSCVVSGFTFSSFGMHWVRQAPDKGLEWVAVIWYDGSNDYYSDSVKGRFTISRDNSKNTLFLQMNRLRAEDTAVYYCARANNWNRFDAFDLWGQGTMVTVSS

SEQ ID NO: 19 - DNA, 24 bp, Homo sapiens
  source 1..24: mol_type = other DNA
ggattcaccttcagcagctttggc

SEQ ID NO: 20 - protein, 8 aa, Homo sapiens
GFTFSSFG

SEQ ID NO: 21 - DNA, 24 bp, Homo sapiens
  source 1..24: mol_type = other DNA
atatggtatgatggaagtaatgat

SEQ ID NO: 22 - protein, 8 aa, Homo sapiens
IWYDGSND

SEQ ID NO: 23 - DNA, 42 bp, Homo sapiens
  source 1..42: mol_type = other DNA
gcgcgagctaataactggaaccgttttgatgcctttgatctc

SEQ ID NO: 24 - protein, 14 aa, Homo sapiens
ARANNWNRFDAFDL

SEQ ID NO: 25 - DNA, 372 bp, Homo sapiens
  source 1..372: mol_type = other DNA
gaggtgcagctggtggagtctgggggaggcttggttcagcctggagggtccctgagactctcctgtgcagcctctggattcatcttcagtaattatgaaatgaactgggtccgccaggctccagggaagggactggaatggatttcatacattactagtagtggtaatatgaaatattacgcagactctgtgaagggccgattcaccatctccagagacaacgacaagaattcactgtatctgcaaatgagtagtctgagagtcgaggacacggctgtttattattgtgtgagaggagggcgatttttggagtggttgacctactacgttatggtcgtctggggccaagggaccacggtcaccgtctcctca

SEQ ID NO: 26 - protein, 124 aa, Homo sapiens
EVQLVESGGGLVQPGGSLRLSCAASGFIFSNYEMNWVRQAPGKGLEWISYITSSGNMKYYADSVKGRFTISRDNDKNSLYLQMSSLRVEDTAVYYCVRGGRFLEWLTYYVMVVWGQGTTVTVSS

SEQ ID NO: 27 - DNA, 24 bp, Homo sapiens
  source 1..24: mol_type = other DNA
ggattcatcttcagtaattatgaa

SEQ ID NO: 28 - protein, 8 aa, Homo sapiens
GFIFSNYE

SEQ ID NO: 29 - DNA, 24 bp, Homo sapiens
  source 1..24: mol_type = other DNA
attactagtagtggtaatatgaaa

SEQ ID NO: 30 - protein, 8 aa, Homo sapiens
ITSSGNMK

SEQ ID NO: 31 - DNA, 51 bp, Homo sapiens
  source 1..51: mol_type = other DNA
gtgagaggagggcgatttttggagtggttgacctactacgttatggtcgtc

SEQ ID NO: 32 - protein, 17 aa, Homo sapiens
VRGGRFLEWLTYYVMVV

SEQ ID NO: 33 - DNA, 360 bp, Homo sapiens
  source 1..360: mol_type = other DNA
caggtgcagctggtggagtctgggggaggcgtggtccagcctgggaggtccctgagactctcctgtgcagtgtctggattcaccttcagtagctatggcatgcactgggtccgccaggctccaggcaaggggctggagtgggtggcaaatatttggtatgatggaactaatgattactatccatactccgtgaagggccgattcaccatctccagagacaattcccagaacacactatatctgcaaatgaacagcctgagagccgaggacacggctgtatattactgtgcgagagaggacttcattaactaccggtcttttgactattggggccagggaaccctggtcaccgtctcctca

SEQ ID NO: 34 - protein, 120 aa, Homo sapiens
QVQLVESGGGVVQPGRSLRLSCAVSGFTFSSYGMHWVRQAPGKGLEWVANIWYDGTNDYYPYSVKGRFTISRDNSQNTLYLQMNSLRAEDTAVYYCAREDFINYRSFDYWGQGTLVTVSS

SEQ ID NO: 35 - DNA, 24 bp, Homo sapiens
  source 1..24: mol_type = other DNA
ggattcaccttcagtagctatggc

SEQ ID NO: 36 - protein, 8 aa, Homo sapiens
GFTFSSYG

SEQ ID NO: 37 - DNA, 24 bp, Homo sapiens
  source 1..24: mol_type = other DNA
atttggtatgatggaactaatgat

SEQ ID NO: 38 - protein, 8 aa, Homo sapiens
IWYDGTND

SEQ ID NO: 39 - DNA, 39 bp, Homo sapiens
  source 1..39: mol_type = other DNA
gcgagagaggacttcattaactaccggtcttttgactat

SEQ ID NO: 40 - protein, 13 aa, Homo sapiens
AREDFINYRSFDY

SEQ ID NO: 41 - DNA, 372 bp, Homo sapiens
  source 1..372: mol_type = other DNA
caggtgcagctggtggagtctgggggaggcgtggtccagccagggacgtccctgagactctcctgtgtcgcgtctggattcaccttcagaaattttggaatgcactgggtccgccaggctccaggcaaggggctggagtgggtggcaaatatatggtttgacggaagtaatgagaactatgtcgagtccattcagggccgattcaccatctccagagacaattccaagaacacactgaatctgcagatgaacagcctgagagccgaggactcggctgtctattactgtgtgagagagggaatcctaggaactactaatccttatgatgcttttgatgtctggggccaagggacaatggtcaccgtctcttca

SEQ ID NO: 42 - protein, 124 aa, Homo sapiens
QVQLVESGGGVVQPGTSLRLSCVASGFTFRNFGMHWVRQAPGKGLEWVANIWFDGSNENYVESIQGRFTISRDNSKNTLNLQMNSLRAEDSAVYYCVREGILGTTNPYDAFDVWGQGTMVTVSS

SEQ ID NO: 43 - DNA, 24 bp, Homo sapiens
  source 1..24: mol_type = other DNA
ggattcaccttcagaaattttgga

SEQ ID NO: 44 - protein, 8 aa, Homo sapiens
GFTFRNFG

SEQ ID NO: 45 - DNA, 24 bp, Homo sapiens
  source 1..24: mol_type = other DNA
atatggtttgacggaagtaatgag

SEQ ID NO: 46 - protein, 8 aa, Homo sapiens
IWFDGSNE

SEQ ID NO: 47 - DNA, 51 bp, Homo sapiens
  source 1..51: mol_type = other DNA
gtgagagagggaatcctaggaactactaatccttatgatgcttttgatgtc

SEQ ID NO: 48 - protein, 17 aa, Homo sapiens
VREGILGTTNPYDAFDV

SEQ ID NO: 49 - DNA, 378 bp, Homo sapiens
  source 1..378: mol_type = other DNA
caggtgcagctggtggagtctgggggaggcgtggtccagcctgggaggtccctgagactctcctgtgcagcgtctggattcacctttagtaactttggaatgcactgggtccgccaggcgccaggcaagggactggagtgggtggcaggtatatggtttgatggaagtaataaaaactatatagactccgtgaagggccgattcaccatctcaagagacaattccaagaacacgctgtatctgcaaatgaacagcctgagagccgaggacacggctgtgtattactgtgcgagagagggctatgattcggggactgattatatcccctatgatatttttgatatttggggccaagggacaatggtcaccgtctcttca

SEQ ID NO: 50 - protein, 126 aa, Homo sapiens
QVQLVESGGGVVQPGRSLRLSCAASGFTFSNFGMHWVRQAPGKGLEWVAGIWFDGSNKNYIDSVKGRFTISRDNSKNTLYLQMNSLRAEDTAVYYCAREGYDSGTDYIPYDIFDIWGQGTMVTVSS

SEQ ID NO: 51 - DNA, 24 bp, Homo sapiens
  source 1..24: mol_type = other DNA
ggattcacctttagtaactttgga

SEQ ID NO: 52 - protein, 8 aa, Homo sapiens
GFTFSNFG

SEQ ID NO: 53 - DNA, 24 bp, Homo sapiens
  source 1..24: mol_type = other DNA
atatggtttgatggaagtaataaa

SEQ ID NO: 54 - protein, 8 aa, Homo sapiens
IWFDGSNK

SEQ ID NO: 55 - DNA, 57 bp, Homo sapiens
  source 1..57: mol_type = other DNA
gcgagagagggctatgattcggggactgattatatcccctatgatatttttgatatt

SEQ ID NO: 56 - protein, 19 aa, Homo sapiens
AREGYDSGTDYIPYDIFDI

SEQ ID NO: 57 - DNA, 363 bp, Homo sapiens
  source 1..363: mol_type = other DNA
gaagtgcagctggtggagtctgggggaggcttggtacagcctggcacgtccctgagactctcctgtgcagcctctggattcacctttgatgattatgccatgcactgggtccggcaagctccagggaagggcctggagtgggtctcaggtattacttggaatagttataacatagactatgctgactctgtgaagggccgattcaccatctccagagacaacgccaagaactccctgtatctgcaaatgaacagtctgagagctgaggacacggccttgtattactgtgcaaaagatgatgactacagtaactacgtttactttgactactggggccagggaaccctggtcaccgtctcctca

SEQ ID NO: 58 - protein, 121 aa, Homo sapiens
EVQLVESGGGLVQPGTSLRLSCAASGFTFDDYAMHWVRQAPGKGLEWVSGITWNSYNIDYADSVKGRFTISRDNAKNSLYLQMNSLRAEDTALYYCAKDDDYSNYVYFDYWGQGTLVTVSS

SEQ ID NO: 59 - DNA, 24 bp, Homo sapiens
  source 1..24: mol_type = other DNA
ggattcacctttgatgattatgcc

SEQ ID NO: 60 - protein, 8 aa, Homo sapiens
GFTFDDYA

SEQ ID NO: 61 - DNA, 24 bp, Homo sapiens
  source 1..24: mol_type = other DNA
attacttggaatagttataacata

SEQ ID NO: 62 - protein, 8 aa, Homo sapiens
ITWNSYNI

SEQ ID NO: 63 - DNA, 42 bp, Homo sapiens
  source 1..42: mol_type = other DNA
gcaaaagatgatgactacagtaactacgtttactttgactac

SEQ ID NO: 64 - protein, 14 aa, Homo sapiens
AKDDDYSNYVYFDY

SEQ ID NO: 65 - DNA, 360 bp, Homo sapiens
  source 1..360: mol_type = other DNA
caggttcagctggtgcagtccggaactgaggtgaaggagcctggggcctcagtgaaggtctcctgtaaggcctctggttactcctttaccacctatggtatcagctggctgcgacaggcccctggacaaggacttgagtggatgggatggatcagcacttacaatggtgacacaatctctgcacagatgctccaggacagagtcaccctgaccgcagacacatccacgcgcacagcctacatggaactgagaagcctgagatctgacgacacggccgtgtattactgtgcgagaggtcatgagtatgatagtcttgtttattcttactggggccagggaaccctggtcaccgtctcctca

SEQ ID NO: 66 - protein, 120 aa, Homo sapiens
QVQLVQSGTEVKEPGASVKVSCKASGYSFTTYGISWLRQAPGQGLEWMGWISTYNGDTISAQMLQDRVTLTADTSTRTAYMELRSLRSDDTAVYYCARGHEYDSLVYSYWGQGTLVTVSS

SEQ ID NO: 67 - DNA, 24 bp, Homo sapiens
  source 1..24: mol_type = other DNA
ggttactcctttaccacctatggt

SEQ ID NO: 68 - protein, 8 aa, Homo sapiens
GYSFTTYG

SEQ ID NO: 69 - DNA, 24 bp, Homo sapiens
  source 1..24: mol_type = other DNA
atcagcacttacaatggtgacaca

SEQ ID NO: 70 - protein, 8 aa, Homo sapiens
ISTYNGDT

SEQ ID NO: 71 - DNA, 39 bp, Homo sapiens
  source 1..39: mol_type = other DNA
gcgagaggtcatgagtatgatagtcttgtttattcttac

SEQ ID NO: 72 - protein, 13 aa, Homo sapiens
ARGHEYDSLVYSY

SEQ ID NO: 73 - DNA, 360 bp, Homo sapiens
  source 1..360: mol_type = other DNA
caggtgcagctggtggagtctgggggaggcgtggtccagcctgggaggtccctgagactctcctgtgcagcgtctggattcaccttcagtagttatgccatgcactgggtccgccaggctccaggcagggggctggagtgggtggcggttatatggcatgatggagatgttgaatactatgtagactccgtgaaggaccgattcaccatctccagagacaattccaagagcacgctgtatctgcaaatgaacagcctgagagccgaagatacggctttatattattgtgcgagagaggcgtgggacctactacgtccctttgactattggggccagggaaccctggtcaccgtctcctca

SEQ ID NO: 74 - protein, 120 aa, Homo sapiens
QVQLVESGGGVVQPGRSLRLSCAASGFTFSSYAMHWVRQAPGRGLEWVAVIWHDGDVEYYVDSVKDRFTISRDNSKSTLYLQMNSLRAEDTALYYCAREAWDLLRPFDYWGQGTLVTVSS

SEQ ID NO: 75 - DNA, 24 bp, Homo sapiens
  source 1..24: mol_type = other DNA
ggattcaccttcagtagttatgcc

SEQ ID NO: 76 - protein, 8 aa, Homo sapiens
GFTFSSYA

SEQ ID NO: 77 - DNA, 24 bp, Homo sapiens
  source 1..24: mol_type = other DNA
atatggcatgatggagatgttgaa

SEQ ID NO: 78 - protein, 8 aa, Homo sapiens
IWHDGDVE

SEQ ID NO: 79 - DNA, 39 bp, Homo sapiens
  source 1..39: mol_type = other DNA
gcgagagaggcgtgggacctactacgtccctttgactat

SEQ ID NO: 80 - protein, 13 aa, Homo sapiens
AREAWDLLRPFDY

SEQ ID NO: 81 - DNA, 351 bp, Homo sapiens
  source 1..351: mol_type = other DNA
gaggtgcagctggtggagtctggaggaggcttggtccagcctggggggtccctgagactctcctgtgcagcctctgggttcatcgtcaccaccaactacatgacctggctccgccaggctccagggaaggggctggagtgggtctcacttatttatagcagtggtcacacatactacgcagactccgtgaagggccgattcaccatctccagacacaattccaagaacacactgtatctacaaatggacagcctgagagctgaggacacggccgtgtattactgtgcgagtgctttcgcagcggatgtttttgatatctggggccaagggacaatggtcaccgtctcttca

SEQ ID NO: 82 - protein, 117 aa, Homo sapiens
EVQLVESGGGLVQPGGSLRLSCAASGFIVTTNYMTWLRQAPGKGLEWVSLIYSSGHTYYADSVKGRFTISRHNSKNTLYLQMDSLRAEDTAVYYCASAFAADVFDIWGQGTMVTVSS

SEQ ID NO: 83 - DNA, 24 bp, Homo sapiens
  source 1..24: mol_type = other DNA
gggttcatcgtcaccaccaactac

SEQ ID NO: 84 - protein, 8 aa, Homo sapiens
GFIVTTNY

SEQ ID NO: 85 - DNA, 21 bp, Homo sapiens
  source 1..21: mol_type = other DNA
atttatagcagtggtcacaca

SEQ ID NO: 86 - protein, 7 aa, Homo sapiens
IYSSGHT

SEQ ID NO: 87 - DNA, 33 bp, Homo sapiens
  source 1..33: mol_type = other DNA
gcgagtgctttcgcagcggatgtttttgatatc

SEQ ID NO: 88 - protein, 11 aa, Homo sapiens
ASAFAADVFDI

SEQ ID NO: 89 - DNA, 372 bp, Homo sapiens
  source 1..372: mol_type = other DNA
caggtgcagctggtggagtctgggggaggcgtggtccagcctgggaggtccctgagactctcctgttcagcgtctggattctccttcagtcactttggcatgcactgggtccgccaggttccaggcgggggcctggagtgggtgacaagtatatggtttgatggaagtaatagatattatgcagactccttgaagggccgattcaccatctccagagacaattccaagaatactctgtatctggaaatgaacagcctgagagccgaggacacggctgtgtattactgtgtgagagaggggatactgggaactactaatccttatgatgtttttgatgtctggggtcaggggacaatggtcaccgtctcttca

SEQ ID NO: 90 - protein, 124 aa, Homo sapiens
QVQLVESGGGVVQPGRSLRLSCSASGFSFSHFGMHWVRQVPGGGLEWVTSIWFDGSNRYYADSLKGRFTISRDNSKNTLYLEMNSLRAEDTAVYYCVREGILGTTNPYDVFDVWGQGTMVTVSS

SEQ ID NO: 91 - DNA, 24 bp, Homo sapiens
  source 1..24: mol_type = other DNA
ggattctccttcagtcactttggc

SEQ ID NO: 92 - protein, 8 aa, Homo sapiens
GFSFSHFG

SEQ ID NO: 93 - DNA, 24 bp, Homo sapiens
  source 1..24: mol_type = other DNA
atatggtttgatggaagtaataga

SEQ ID NO: 94 - protein, 8 aa, Homo sapiens
IWFDGSNR

SEQ ID NO: 95 - DNA, 51 bp, Homo sapiens
  source 1..51: mol_type = other DNA
gtgagagaggggatactgggaactactaatccttatgatgtttttgatgtc

SEQ ID NO: 96 - protein, 17 aa, Homo sapiens
VREGILGTTNPYDVFDV

SEQ ID NO: 97 - DNA, 372 bp, Homo sapiens
  source 1..372: mol_type = other DNA
gaggtgcagctggtggagtctgggggaggcttggtacagccgggggggtccctgagactctcctgtgcagcctctggattcacctttagaagctatgtcatgagctggttccgccaggctccagggaaggggctggagtgggtctcaggaatgagtgggagtggtggaagcacatcctacgcagactccgtgaagggccggttcaccatctccagagacaattcaaagaatacgctgtatctgctaatgaacagcctgagaaccgaggacacggccgtatattattgtgcgaaagaaaacggggctaactggaactacggctactacggtatggacgtctggggccaagggaccacggtcaccgtctcctca

SEQ ID NO: 98 - protein, 124 aa, Homo sapiens
EVQLVESGGGLVQPGGSLRLSCAASGFTFRSYVMSWFRQAPGKGLEWVSGMSGSGGSTSYADSVKGRFTISRDNSKNTLYLLMNSLRTEDTAVYYCAKENGANWNYGYYGMDVWGQGTTVTVSS

SEQ ID NO: 99 - DNA, 24 bp, Homo sapiens
  source 1..24: mol_type = other DNA
ggattcacctttagaagctatgtc

SEQ ID NO: 100 - protein, 8 aa, Homo sapiens
GFTFRSYV

SEQ ID NO: 101 - DNA, 24 bp, Homo sapiens
  source 1..24: mol_type = other DNA
atgagtgggagtggtggaagcaca

SEQ ID NO: 102 - protein, 8 aa, Homo sapiens
MSGSGGST

SEQ ID NO: 103 - DNA, 51 bp, Homo sapiens
  source 1..51: mol_type = other DNA
gcgaaagaaaacggggctaactggaactacggctactacggtatggacgtc

SEQ ID NO: 104 - protein, 17 aa, Homo sapiens
AKENGANWNYGYYGMDV

SEQ ID NO: 105 - DNA, 360 bp, Homo sapiens
  source 1..360: mol_type = other DNA
caggtgcagctggtggagtctgggggaggcgtggtccagcctgggaggtccctgagactctcctgtgtagcgtctggattctccttcagtaactttggcatgcactgggtccgccaggctccaggcaaggggctggagtgggtggcaattatatggtatgatggaagtaataaatactattcagactccgtgaagggccgcttcaccatctccagagacaattccaagaacacgctgtatctgcaaatgaacagcctgagagtcgacgacacggctgtgtattactgtgcgagattcgatcgctggaaatttgacgcttttgatatctggggccaagggacaatggtcaccgtctcttca

SEQ ID NO: 106 - protein, 120 aa, Homo sapiens
QVQLVESGGGVVQPGRSLRLSCVASGFSFSNFGMHWVRQAPGKGLEWVAIIWYDGSNKYYSDSVKGRFTISRDNSKNTLYLQMNSLRVDDTAVYYCARFDRWKFDAFDIWGQGTMVTVSS

SEQ ID NO: 107 - DNA, 24 bp, Homo sapiens
  source 1..24: mol_type = other DNA
ggattctccttcagtaactttggc

SEQ ID NO: 108 - protein, 8 aa, Homo sapiens
GFSFSNFG

SEQ ID NO: 109 - DNA, 24 bp, Homo sapiens
  source 1..24: mol_type = other DNA
atatggtatgatggaagtaataaa

SEQ ID NO: 110 - protein, 8 aa, Homo sapiens
IWYDGSNK

SEQ ID NO: 111 - DNA, 39 bp, Homo sapiens
  source 1..39: mol_type = other DNA
gcgagattcgatcgctggaaatttgacgcttttgatatc

SEQ ID NO: 112 - protein, 13 aa, Homo sapiens
ARFDRWKFDAFDI

SEQ ID NO: 113 - DNA, 360 bp, Homo sapiens
  source 1..360: mol_type = other DNA
caggtgcagctggtggagtctgggggaggcgtggtccagcctgggaggtccctgagactctcctgtgcagcgtctggattcaccttcagtagctttgccatgcactgggtccgccaggctccaggcaaggggctggagtgggtggcagttatatggtatgatggaagtaatgattactatgcagcctccgtgaagggccgtttcaccatctccagagacaattccaagaacacgctgtatctggaaatgaacagactgagagccgaggacacggctgtgtatcactgtgcgagagataactggaattactgggggggtatggacgtctggggccaagggaccacggtcaccgtctcctca

SEQ ID NO: 114 - protein, 120 aa, Homo sapiens
QVQLVESGGGVVQPGRSLRLSCAASGFTFSSFAMHWVRQAPGKGLEWVAVIWYDGSNDYYAASVKGRFTISRDNSKNTLYLEMNRLRAEDTAVYHCARDNWNYWGGMDVWGQGTTVTVSS

SEQ ID NO: 115 - DNA, 24 bp, Homo sapiens
  source 1..24: mol_type = other DNA
ggattcaccttcagtagctttgcc

SEQ ID NO: 116 - protein, 8 aa, Homo sapiens
GFTFSSFA

SEQ ID NO: 117 - DNA, 24 bp, Homo sapiens
  source 1..24: mol_type = other DNA
atatggtatgatggaagtaatgat

SEQ ID NO: 118 - protein, 8 aa, Homo sapiens
IWYDGSND

SEQ ID NO: 119 - DNA, 39 bp, Homo sapiens
  source 1..39: mol_type = other DNA
gcgagagataactggaattactgggggggtatggacgtc

SEQ ID NO: 120 - protein, 13 aa, Homo sapiens
ARDNWNYWGGMDV

SEQ ID NO: 121 - DNA, 375 bp, Homo sapiens
  source 1..375: mol_type = other DNA
gaggtgcagctgttggagtctgggggaggctgggtgcagccgggggggtccctgagactctcctgtgcagcctctggattcgcctttagtaattatgccatgaactgggtccgccagactccagggaaggggctggagtgggtctcagttattagtagtagtggtggaaacacatactacgcagactccgtgaagggccggttcgccatctccagagacaattccagggatacgctgcatctgcaaatgaacagactgagagtcgaggacacggccgtctattactgtgcgaaagaaatacgtccgtattacgatctttcctactattacggtatggacgtctggggccaagggaccacggtcaccgtctcctca

SEQ ID NO: 122 - protein, 125 aa, Homo sapiens
EVQLLESGGGWVQPGGSLRLSCAASGFAFSNYAMNWVRQTPGKGLEWVSVISSSGGNTYYADSVKGRFAISRDNSRDTLHLQMNRLRVEDTAVYYCAKEIRPYYDLSYYYGMDVWGQGTTVTVSS

SEQ ID NO: 123 - DNA, 24 bp, Homo sapiens
  source 1..24: mol_type = other DNA
ggattcgcctttagtaattatgcc

SEQ ID NO: 124 - protein, 8 aa, Homo sapiens
GFAFSNYA

SEQ ID NO: 125 - DNA, 24 bp, Homo sapiens
  source 1..24: mol_type = other DNA
attagtagtagtggtggaaacaca

SEQ ID NO: 126 - protein, 8 aa, Homo sapiens
ISSSGGNT

SEQ ID NO: 127 - DNA, 54 bp, Homo sapiens
  source 1..54: mol_type = other DNA
gcgaaagaaatacgtccgtattacgatctttcctactattacggtatggacgtc

SEQ ID NO: 128 - protein, 18 aa, Homo sapiens
AKEIRPYYDLSYYYGMDV

SEQ ID NO: 129 - DNA, 348 bp, Homo sapiens
  source 1..348: mol_type = other DNA
caggtgcagctgcaggagtcgggcccaggactggtgaagtcttcggagaccctgtccctcacctgcactgtctctggtggctccatcagaaatttctactggagctggctccggcagcccccagggaagggactagagtggattgggcacatcaattacaatgggggcaccgactacaacccctccctcaagagtcgagtcaccatatcagtagacacgtccaagaatcagttctccctgaatttgaactctgtgaccgccgcagacacggccgtgtattactgtgcgagacagagattctacggtatggacgtctggggtccagggaccacggtcaccgtctcctca

SEQ ID NO: 130 - protein, 116 aa, Homo sapiens
QVQLQESGPGLVKSSETLSLTCTVSGGSIRNFYWSWLRQPPGKGLEWIGHINYNGGTDYNPSLKSRVTISVDTSKNQFSLNLNSVTAADTAVYYCARQRFYGMDVWGPGTTVTVSS

SEQ ID NO: 131 - DNA, 24 bp, Homo sapiens
  source 1..24: mol_type = other DNA
ggtggctccatcagaaatttctac

SEQ ID NO: 132 - protein, 8 aa, Homo sapiens
GGSIRNFY

SEQ ID NO: 133 - DNA, 21 bp, Homo sapiens
  source 1..21: mol_type = other DNA
atcaattacaatgggggcacc

SEQ ID NO: 134 - protein, 7 aa, Homo sapiens
INYNGGT

SEQ ID NO: 135 - DNA, 30 bp, Homo sapiens
  source 1..30: mol_type = other DNA
gcgagacagagattctacggtatggacgtc

SEQ ID NO: 136 - protein, 10 aa, Homo sapiens
ARQRFYGMDV

SEQ ID NO: 137 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 138 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 139 - DNA, 18 bp, Homo sapiens
  source 1..18: mol_type = other DNA
cagagcattagcagctat

SEQ ID NO: 140 - protein, 6 aa, Homo sapiens
QSISSY

SEQ ID NO: 141-142 - skipped (2 entries)

SEQ ID NO: 143 - DNA, 30 bp, Homo sapiens
  source 1..30: mol_type = other DNA
caacagagttacagtacccctccgatcacc

SEQ ID NO: 144 - protein, 10 aa, Homo sapiens
QQSYSTPPIT

SEQ ID NO: 145 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 146 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 147 - protein, 934 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 148 - protein, 281 aa, Homo sapiens
KEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKR

SEQ ID NO: 149 - protein, 1101 aa, Homo sapiens
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 150 - protein, 1082 aa, Homo sapiens
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 151 - protein, 626 aa, Homo sapiens
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 152 - protein, 650 aa, synthetic construct
  REGION 1..650: note = synthetic
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAEQKLISEEDLEQKLISEEDLHHHHHH

SEQ ID NO: 153 - protein, 838 aa, synthetic construct
  REGION 1..838: note = synthetic
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAMEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKE

SEQ ID NO: 154 - protein, 1100 aa, synthetic construct
  REGION 1..1100: note = synthetic
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTEQKLISEEDLEQKLISEEDLHHHHHH

SEQ ID NO: 155 - protein, 936 aa, synthetic construct
  REGION 1..936: note = synthetic
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTEQKLISEEDLGGEQKLISEEDLHHHHHH

SEQ ID NO: 156 - protein, 12 aa, synthetic construct
  REGION 1..12: note = synthetic
VRRLKETKDGFM

SEQ ID NO: 157 - protein, 11 aa, synthetic construct
  REGION 1..11: note = synthetic
LARQIGASLND

SEQ ID NO: 158 - protein, 35 aa, synthetic construct
  REGION 1..35: note = synthetic
FIKGDLTIANLGTSEGRFMQVVVSRSGPSTPHVNF